(19) United States
(12) Patent Application Publication
(10) Pub. No.: US 2011/0105565 A1
(43) Pub. Date: May 5, 2011

(54) WATER SOLUBLE SMALL MOLECULE INHIBITORS OF THE CYSTIC FIBROSIS TRANSMEMBRANE CONDUCTANCE REGULATOR

(75) Inventors: Alan S. Verkman, San Francisco, CA (US); Nitin D. Sonawane, San Francisco, CA (US)

(73) Assignee: THE REGENTS OF THE UNIVERSITY OF CALIFORNIA, Oakland, CA (US)

(21) Appl. No.: 12/934,621

(22) PCT Filed: Mar. 25, 2009
(86) PCT No.: PCT/US09/38292

§ 371 Date: Jan. 21, 2011

Related U.S. Application Data
(60) Provisional application No. 61/039,379, filed on Mar. 25, 2008.
(60) Provisional application No. 61/084,228, filed on Jul. 28, 2008.

(51) Int. Cl. A61K 31/4439; C07D 277/08; C07D 417/06; C07D 207/456; A61K 31/427; A61K 31/426; A61P 13/12

(52) U.S. Cl. 514/336; 548/183; 546/269.7; 548/544; 514/369

(57) ABSTRACT

Provided herein are highly water soluble, thiazolidinone derivative compounds and glycine hydrazide derivative compounds that inhibit the ion transport activity of the cystic fibrosis transmembrane conductance regulator (CFTR). The compounds, and compositions comprising the compounds, described herein are useful for treating diseases, disorders, and sequelae of diseases, disorders, and conditions that are associated with aberrantly increased CFTR activity, for example, secretory diarrhea. The compounds may also be used for inhibiting expansion or preventing formation of cysts in persons who have polycystic kidney disease.

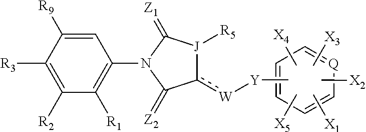

### CROSS-REFERENCE TO RELATED APPLICATIONS

This application claims the benefit of U.S. Provisional Application No. 61/039,379 filed Mar. 25, 2008 and U.S. Provisional Application No. 61/084,228 filed Jul. 28, 2008, both of which are incorporated herein by reference in their entireties.

### STATEMENT OF GOVERNMENT INTEREST

This invention was made with government support under grants HL73856, DK72517, EB00415, HL59198, DK35124, EY13574, and HL73854, and DK43840 awarded by National Institutes of Health. The government has certain rights in this invention.

### BACKGROUND

1. Field

Therapeutics are needed for treating diseases and disorders related to aberrant cystic fibrosis transmembrane conductance regulator protein (CFTR)-mediated ion transport, such as increased intestinal fluid secretion, secretory diarrhea, and polycystic kidney disease. Small molecule compounds are described herein that are potent inhibitors of CFTR activity and that may be used for treating such diseases and disorders.

2. Description of the Related Art

The cystic fibrosis transmembrane conductance regulator protein (CFTR) is a cAMP-activated chloride (Cl⁻) channel expressed in epithelial cells in mammalian airways, intestine, pancreas and testis. CFTR is the chloride-channel responsible for cAMP-mediated Cl⁻ secretion. Hormones, such as a β-adrenergic agonist, or a toxin, such as cholera toxin, leads to an increase in cAMP, activation of cAMP-dependent protein kinase, and phosphorylation of the CFTR Cl channel, which causes the channel to open. An increase in cell Ca²⁺ can also activate different apical membrane channels. Phosphorylation by protein kinase C can either open or shut Cl channels in the apical membrane. CFTR is predominantly located in epithelia where it provides a pathway for the movement of Cl⁻ ions across the apical membrane and a key point at which to regulate the rate of transepithelial salt and water transport.

CFTR chloride channel function is associated with a wide spectrum of disease, including cystic fibrosis (CF) and with some forms of male infertility, polycystic kidney disease and secretory diarrhea. Cystic fibrosis is a hereditary lethal disease caused by mutations in CFTR (see, e.g., Quinton, Physiol. Rev. 79:S3-S22 (1999); Boucher, Eur. Respir. J. 23:146-58 (2004)). Observations in human patients with CF and mouse models of CF indicate the functional importance of CFTR in intestinal and pancreatic fluid transport, as well as in male fertility (Grubb et al., Physiol. Rev. 79:S193-S214 (1999); Wong, P. Y., Mol. Hum. Reprod. 4:107-110 (1997)). CFTR is expressed in enterocytes in the intestine and in cyst epithelium in polycystic kidney disease (see, e.g., O'Sullivan et al., Am. J. Kidney Dis. 32:976-983 (1998); Sullivan et al., Physiol. Rev. 78:1165-91 (1998); Strong et al., J. Clin. Invest. 93:347-54 (1994); Mall et al., Gastroenterology 126:32-41 (2004); Hanaoka et al., Am. J. Physiol. 270:C389-C399 (1996); Kunzelmann et al., Physiol. Rev. 82:245-289 (2002); Davidow et al., Kidney Int. 50:208-18 (1996); Li et al., Kidney Int. 66:1926-38 (2004); Al-Awqati, J. Clin. Invest. 110:1599-1601 (2002); Thiagarajah et al., Curr. Opin. Pharmacol. 3:594-99 (2003)).

High-affinity CFTR inhibitors have clinical applications in the therapy of secretory diarrheas. Cell culture and animal models indicate that intestinal chloride secretion in enterotoxin-mediated secretory diarrheas occurs mainly through the CFTR (see, e.g., Clarke et al., Science 257:1125-28 (1992); Gabriel et al., Science 266:107-109 (1994); Kunzelmann and Mall, Physiol. Rev. 82:245-89 (2002); Field, M. J. Clin. Invest. 111:931-43 (2003); and Thiagarajah et al., Gastroenterology 126:511-519 (2003)).

Diarrheal disease in children is a global health concern: approximately four billion cases among children occur annually, resulting in at least two million deaths. Travelers' diarrhea affects approximately 6 million people per year. Antibiotics are routinely used to treat diarrhea; however, the antibiotics are ineffective for treating many pathogens, and the use of these drugs contributes to development of antibiotic resistance in other pathogens.

Oral replacement of fluid loss is also routinely used to treat diarrhea, but is primarily palliative. Therapy directed at reducing intestinal fluid secretion (anti-secretory therapy') has the potential to overcome limitations of existing therapies.

Polycystic kidney disease (PKD) is characterized by massive enlargement of fluid-filled cysts of renal tubular origin that compromise normal renal parenchyma and cause renal failure (Arnaout, Annu Rev Med 52: 93-123, 2001; Gabow N Engl J Med 329: 332-342, 1993; Harris et al., Mol Genet Metab 81: 75-85, 2004; Wilson N Engl J Med 350: 151-164, 2004; Sweeney et al., Cell Tissue Res 326: 671-685, 2006; Chapman J Am Soc Nephrol 18: 1399-1407, 2007). Human autosomal dominant PKD (ADPKD) is caused by mutations in one of two genes, PKD1 and PKD2, encoding the interacting proteins polycystin-1 and polycystin-2, respectively (Wilson, supra; Qian et al., Cell 87: 979-987, 1996; Wu et al., Cell 93: 177-188, 1998; Watnick et al., Tones et al., Nat Med 10: 363-364, 2004 Nat Genet. 25: 143-144, 2000). Cyst growth in PKD involves fluid secretion into the cyst lumen coupled with epithelial cell hyperplasia.

Several CFTR inhibitors have been discovered, although many exhibit weak potency and lack CFTR specificity. The oral hypoglycemic agent glibenclamide inhibits CFTR Cl conductance from the intracellular side by an open channel blocking mechanism (Sheppard & Robinson, J. Physiol., 503:333-346 (1997); Zhou et al., J. Gen. Physiol. 120:647-62 (2002)) at high micromolar concentrations where it affects other Cl and cation channels (Edwards & Weston, 1993; Rabe et al., Br. J. Pharmacol. 110:1280-81 (1995); Schultz et al., Physiol. Rev. 79:S109-S144 (1999)). Other non-selective anion transport inhibitors including diphenylamine-2-carboxylate (DPC), 5-nitro-2(3-phenylpropyl-amino)benzoate (NPPB), and flufenamic acid also inhibit CFTR by occluding the pore at an intracellular site (Dawson et al., Physiol. Rev., 79:S47-S75 (1999); McCarty, J. Exp. Biol., 203:1947-62 (2000)).

A need exists for CFTR inhibitors, particularly those that are safe, non-absorbable, highly potent, inexpensive, and chemically stable.

### BRIEF SUMMARY

Briefly, provided herein are thiazolidinone compounds and glycine hydrazide compounds, and compositions comprising such compounds, that inhibit cystic fibrosis transmembrane conductance regulator (CFTR) mediated ion transport and that are useful for treating diseases and disorders associated with aberrantly increased CFTR chloride channel activity. Methods of treating diseases and disorders associated with aberrantly increased intestinal fluid secretion are provided. Also provided are methods of inhibiting enlargement or preventing formation of cysts and thereby treating polycystic kidney disease. The thiazolidinone compounds and glycine hydrazide compounds described herein have improved water solubility properties that contribute to the therapeutic effectiveness of the compounds for use in treating diseases and disorders associated with aberrantly increased CFTR chloride channel activity.

In one embodiment, thiazolidinone derivative compounds of structure I, which have the following formula are provided:

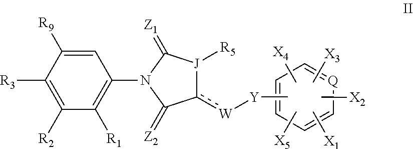

or a pharmaceutically acceptable salt, prodrug, or stereoisomer thereof, wherein

Y is —NH— or absent;

W is ═CH—, —S—, —O—, —C(═S)—, or —C(═O)—;

Z₁, Z₂, Z₃, Z₄, and Z₅ are each independently O or S;

J is C, S, O, or N;

Q is C or N;

R₁, R₂, R₃, and R₉ are each independently H, C₁₋₆ alkyl, alkoxy, halo, —CF₃, —CF₂CF₃, or —OCF₃;

R₅ is H, halo, C₁₋₆ alkyl, or absent;

X₁, X₂, X₃, and X₄ are each independently H, —OH, —SH, halo, tetrazolo, —P(═O)(OH)₂, —C(═Z₃)Z₄H, —Z₅—C(═Z₃)Z₄H, or —Z₅—CH₂—C(═Z₃)Z₄H; and

X₅ is —O⁻, tetrazolo, —C(═O)OH, —O—C(═O)OH, or absent.

Also provided herein are substructures of thiazolidinone derivative compounds having formulae I(A), I(A1-A8), I(B), I(B1), I(C), and I(C1) as described in greater detail herein.

In another embodiment, glycine hydrazide derivative compounds having a structure II of the following formula are provided:

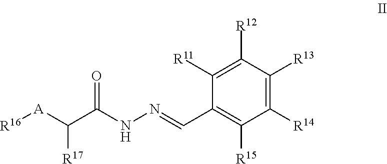

or a pharmaceutically acceptable salt, prodrug, or stereoisomer thereof, wherein

A is —O— or —NH—;

R¹¹, R¹², R¹³, R¹⁴, R¹⁵ are each the same or different and independently hydrogen, hydroxy, C₁₋₆ alkyl, C₁₋₆ alkoxy, carboxy, halo, nitro, aryl, and heteroaryl;

R¹⁶ is phenyl, heteroaryl, quinolinyl, anthracenyl, or naphthalenyl; and

R¹⁷ is H, alkoxy, or substituted or unsubstituted aryl.

In other embodiments, also provided are substructures of glycine hydrazide compounds of formula II, which have a structure of formulae II(A) or II(B), which are described in greater detail herein.

Also provided herein are methods of preparing compounds of structure I and II (and substructures thereof), pharmaceutical preparations of the same, and methods for inhibiting the cystic fibrosis transmembrane conductance regulator (CFTR) chloride channel, and for treating diseases, disorders, and conditions associated with aberrantly increased CFTR activity. In other embodiments, methods for inhibiting cyst formation or cyst enlargement, and for treating polycystic kidney disease are provided.

In certain embodiments, pharmaceutical compositions are provided, wherein the pharmaceutical composition comprises a pharmaceutically suitable excipient and a compound (I.e., at least one compound) of any one of the structures, substructures, and specific compounds described herein, including a thiazolidinone derivative compound having a structure of formulae I, I(A), I(A1-A8), I(B), I(B1), I(C), I(C1), as described above and in greater detail herein. Also provided are pharmaceutical compositions wherein the pharmaceutical composition comprises a pharmaceutically suitable excipient and a compound (I.e., at least one compound) having any one of the structures, substructures, and specific compound structures described herein, including a glycine hydrazide derivative compound having a structure of formulae II, II(A), and II(B) as described in detail above and herein.

In other embodiments, pharmaceutical compositions are provided wherein the pharmaceutical composition comprises a pharmaceutically suitable excipient and at least one of the compounds having any one of the structures, substructures, and specific compounds described in detail herein, including a compound of structure I and substructures I(A), I(A1-A8), I(B), I(B1), I(C), I(C1) and specific structures as described herein (i.e., thiazolidinone derivative compounds) and at least one compound of structure II and substructures II(A) and II(B) and specific structures described in detail herein (i.e., glycine hydrazide derivative compounds).

In other embodiments, a method is provided for inhibiting cyst formation or cyst enlargement comprising contacting (a) a cell that comprises CFTR and (b) at least one compound of structure I and substructures I(A), I(A1-A8), I(B), I(B1), I(C), I(C1) and specific structures as described herein (i.e., thiazolidinone derivative compounds) and/or at least one compound of structure II and substructures II(A) and II(B) and specific structures described herein (i.e., glycine hydrazide derivative compounds), under conditions and for a time sufficient that permit the CFTR and the compound to interact, wherein the compound inhibits ion transport by CFTR.

In yet another embodiment, a method is provided for treating polycystic kidney disease comprising administering to subject a (a) pharmaceutically suitable excipient and (b) at least one of the compounds of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein (i.e., a pharmaceutical composition as described herein). In a specific embodiment, polycystic kidney disease is autosomal dominant polycystic kidney disease. In another specific embodiment, polycystic kidney disease is autosomal recessive polycystic kidney disease.

In another embodiment, a method is provided for treating a disease or disorder associated with aberrantly increased ion transport by cystic fibrosis transmembrane conductance regulator (CFTR), the method comprising administering to a subject a pharmaceutically suitable excipient and at least one of the compounds of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein (i.e., a pharmaceutical composition as described herein), wherein ion transport by CFTR is inhibited. In a specific embodiment, the disease or disorder is aberrantly increased intestinal fluid secretion. In a more specific embodiment, the disease or disorder is secretory diarrhea. In a specific embodiment, secretory diarrhea is caused by an enteric pathogen. In particular embodiments, the enteric pathogen is Vibrio cholerae, Clostridium difficile, Escherichia coli, Shigella, Salmonella, rotavirus, Giardia lamblia, Entamoeba histolytica, Campylobacter jejuni, and Cryptosporidium. In other specific embodiments, the secretory diarrhea is induced by an enterotoxin. In particular embodiments, the enterotoxin is a cholera toxin, a E. coli toxin, a Salmonella toxin, a Campylobacter toxin, or a Shigella toxin. In other particular embodiments, secretory diarrhea is a sequelae of ulcerative colitis, irritable bowel syndrome (IBS), AIDS, chemotherapy, or an enteropathogenic infection.

In particular embodiments of the methods described herein, the subject is a human or non-human animal.

In another embodiment, a method is provided for inhibiting ion transport by a cystic fibrosis transmembrane conductance regulator (CFTR) comprising contacting (a) a cell that comprises CFTR and (b) at least one of the compounds of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein, or a pharmaceutical composition comprising at least one of the compounds, under conditions and for a time sufficient that permit the CFTR and the compound to interact, thereby inhibiting ion transport by CFTR.

In another embodiment, a method is provided for treating secretory diarrhea comprising administering to a subject a pharmaceutically acceptable excipient and at least one of the compounds of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein (i.e., a pharmaceutical composition as described herein). In a particular embodiment, the subject is a human or non-human animal.

In particular embodiments of each of the methods described in detail herein, (including the method of inhibiting cyst formation or cyst enlargement, the method of treating polycystic kidney disease, the method of treating a disease or disorder associated with aberrantly increased ion transport by cystic fibrosis transmembrane conductance regulator (CFTR), method of inhibiting ion transport by CFTR, and the method of treating secretory diarrhea), the method includes use of a compound selected from:

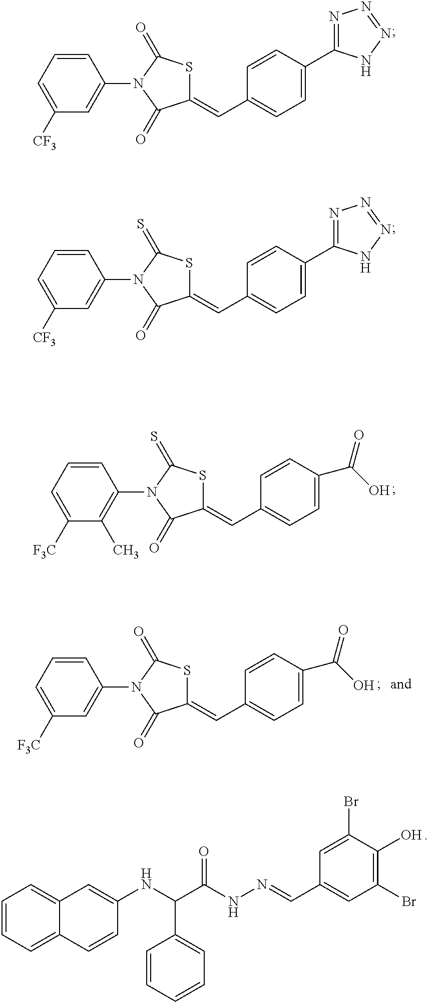

In other embodiments provided herein, is use of a compound of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein, for the manufacture of a pharmaceutical composition for treating a disease or disorder associated with aberrantly increased ion transport by cystic fibrosis transmembrane conductance regulator (CFTR), said disease or disorder selected from polycystic kidney disease, aberrantly increased intestinal fluid secretion, and secretory diarrhea.

As used herein and in the appended claims, the singular forms “a,” “and,” and “the” include plural referents unless the context clearly dictates otherwise. Thus, for example, reference to “an agent” includes a plurality of such agents, and reference to “a cell” or “the cell” includes reference to one or more cells and equivalents thereof (e.g., plurality of cells) known to those skilled in the art, and so forth. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### BRIEF DESCRIPTION OF THE DRAWINGS

FIG. 1 is a schematic of compound CFTRinh-172, a thiazolidinone derivative compound.

FIG. 2A-H shows the effects of CFTR inhibitors on the growth of MDCK cell cysts in cell culture. FIG. 2A provides light micrographs taken at indicated days after cell seeding of MDCK cells exposed continuously to 10 μM forskolin (scale bar, 500 μm) (top). In some experiments, CFTR inhibitor T08 was added for 8 days (middle) or 4 days (bottom), from day 4 onward after cell seeding in gels. FIG. 2B shows cyst inhibition activity of thiazolidionone and glycine hydrazide analogs T1-T16 and G1-G16, as measured by cyst diameter (S.E., n>10). C=CMSO vehicle control; 172=CFTRᵢₙₕ-172. FIG. 2C illustrates the cytotoxicity of certain thiazolidionone and glycine hydrazide analogs T1-T16 and G1-G16, respectively, as assayed by crystal violet staining (S.E., n=3, * P<0.05). C=CMSO vehicle control; 172=CFTRᵢₙₕ-172. FIG. 2D shows MDCK cell cyst growth shown as cyst diameters for indicated compounds (S.E., n>30 cysts analyzed per time point). FIG. 2E shows effects of the indicated compounds on MDCK cell cyst formation; white bars show the total numbers of colonies (including cysts and non-cyst colonies) per well on day 6 after MDCK cell seeding in the absence (control) and presence of test compounds (at 10 μM), and shaded area of bars show the numbers of cysts with diameter >50 μm (S.E., 4 wells per condition, *, P<0.05). FIG. 2F demonstrates inhibition of short-circuit current in MDCK cell monolayer by compounds T08 and G07 after chloride current stimulation by 20 μM forskolin. FIG. 2G (top) illustrates MDCK cell proliferation over 72 hours in the presence of various concentrations of the indicated compounds, as measured by BrdU incorporation (S.E., n=3, * P<0.05); DMSO was used as negative control, and blasticidin (20 μg/ml) was used as positive control. FIG. 2G (bottom) shows MDCK cell apoptosis over 72 hours in the presence of various concentrations of the indicated compounds, as assayed by the detection of fluorescein-dUTP-labeled DNA strand breaks by fluorescence microscopy (S.E., n=5, * P<0.05); DMSO was used as negative control and gentamicin (2 mM) (genta.) was used as positive control. The data in FIG. 2H represent short-circuit current measurements in MDCK cell monolayers cultured without or with 10 μM T08 or G07 for 1 or 48 h. Compounds were washed out for 1 h before measurements, and CFTR chloride current was stimulated by 20 μM forskolin.

FIGS. 3A-3D illustrate the structures of thiazolidinone CFTR inhibitors with their CFTR inhibition activity (IC₅₀ values).

FIGS. 4A-4F depict structures of glycine hydrazide and malonic acid hydrazide CFTR inhibitors with their CFTR inhibition activity (IC₅₀ values).

FIG. 5A-E shows the effects of CFTR inhibitors on cyst growth in embryonic kidney organ cultures. Embryonic kidneys were placed in culture at day E13.5 and maintained for four days in culture. FIG. 5A shows kidney appearance by transmitted light microscopy for cultures in the absence (top) or continued presence (bottom) of 100 μM 8-Br-cAMP. Each series of photographs shows the same kidney on successive days in culture (scale bar, 1 mm). FIG. 5B shows inhibition of cAMP-induced cyst growth by compounds T08 and G07. Images are shown of embryonic kidneys before (day 0) and 4 days after compound addition (scale bar, 1 mm). FIG. 5C illustrates the fractional cyst area in control and CFTR inhibitor-treated kidneys (S.E., n=6-12, * P<0.05, ** P<0.01 vs. control). FIG. 5D shows the reversible inhibition of cyst growth. Compound T08 was added for 2 days (top) or 4 days (bottom) in culture medium containing 100 μM 8-Br cAMP (scale bar, 1 mm). FIG. 5E represents histological staining (H&E staining) of embryonic kidneys in the presence or absence of the indicated compounds (scale bar, 1 mm).

FIG. 6A-C provides liquid chromatography (LC) and mass spectrometry (MS) analysis of inhibitor concentrations in kidney and urine. FIG. 6A shows representative HPLC profile of urine spiked with 50 pM each of tetrazolo-CFTRᵢₙₕ-172 (compound T08, top) and Ph-GlyH-101 (compound G07, bottom) with their respective mass trace profiles of 432 m/z (top inset) and 554 m/z trace (bottom inset), demonstrating assay sensitivity. FIG. 6B provides calibrations of absorbance peak areas (from HPLC) for known amounts of inhibitors added to urine. FIG. 6C indicates urine concentrations at 1 and 5 h after subcutaneous administration at 5-10 mg/kg/day for 3 days (S.E., n=3).

FIG. 7A-D shows the effect of CFTR inhibitors on cyst growth in a Pkd1^{flox/-}; Ksp-Cre mouse model of PKD. FIG. 7A is a gallery of kidney sections from Pkd1^{flox/-}; Ksp-Cre mice treated for 3 days with DMSO vehicle (left panel) or CFTR inhibitors as indicated (10 mg/kg/day, middle and right panels). FIG. 7B shows kidney weights (age 5 days) of non-PKD mice (denoted ‘wild-type’) and Pkd1^{flox/-}; Ksp-Cre mice treated for 3 days with DMSO vehicle (C) or compounds T08 or G07 (S.E., 11 mice per group, * P<0.01). FIG. 7C provides a histogram of cyst numbers at indicated ranges of cyst areas (kidneys from 11 mice analyzed). FIG. 7D represents renal function in CFTR inhibitor-treated Pkd1^{flox/-}; Ksp-Cre mice at age 5 and 9 days. Mice were treated from day 2 onward. Serum creatinine and urea concentrations are shown (S.E., 4 mice per group, * P<0.05 compared to control).

FIG. 8A-F, shows short-circuit current measurements of CFTR inhibition. FIGS. 8A-E show CFTR-mediated apical membrane chloride current measured in FRT cells expressing human wildtype CFTR after permeabilization of the basolateral membrane in the presence of a chloride gradient for CFTRᵢₙₕ-172, Tetrazolo-172, Oxo-172 and α-Me-172 respectively. FIG. 8F shows concentration-inhibition data for CFTRᵢₙₕ-172 (compound 5), Tetrazolo-172 (compound 6), Oxo-172 (compound 47), α-Me-172 (compound 18), and Pyridine-NO-172 (compound 17).

### DETAILED DESCRIPTION

Provided herein are thiazolidinone derivative compounds and glycine hydrazide derivative compounds that inhibit activity of the cystic fibrosis transmembrane conductance regulator (CFTR) chloride channel. The compounds and compositions comprising the compounds described herein have significantly increased water solubility compared with previously identified thiazolidinone and hydrazide compounds. Increased solubility of these compounds in water and saline improves oral bioavailability of the compounds. By way of example, a thiazolidinone derivative compound referred to herein as CFTRᵢₙₕ-172 has relatively low water solubility (less than 17 μM) and low oral bioavailability (see, e.g., Thiagarajah et al., Gastroenterology 126:511-519 (2003); Sonawane et al., J. Pharm. Sci. 94:134-143 (2005); Perez et al., Am. J. Physiol. 292(2, Pt. 1) L383-L395 (2007); Taddei et al., FEBS Lett. 558:52-56 (2004)). Exemplary thiazolidinone compounds described herein exhibited three to twenty-fold increased water solubility.

The thiazolidinone derivative compounds and glycine hydrazide derivative compounds described herein and compositions comprising the compounds may therefore be used for treating diseases and disorders associated with aberrantly increased CFTR-mediated transepithelial fluid secretion. Such diseases and disorders include secretory diarrhea, which may be caused by enteropathogenic organisms including bacteria, viruses, and parasites, such as but not limited to Vibrio cholerae, Clostridium difficile, Escherichia coli, Shigella, Salmonella, rotavirus, Campylobacter jejuni, Giardia lamblia, Entamoeba histolytica, Cyclospora, and Cryptosporidium or by toxins such as cholera toxin and Shigella toxin. The derivative compounds described herein may also be useful for treating secretory diarrhea that is a sequelae of a disease, disorder, or condition, including but not limited to AIDS, administration of AIDS related therapies, chemotherapy, and inflammatory gastrointestinal disorders such as ulcerative colitis, inflammatory bowel disease (IBD), and Crohn's disease.

These compounds and compositions are also useful for inhibiting cyst expansion or enlargement or preventing cyst formation and are thus useful for treating polycystic kidney disease. Polycystic kidney disease (PKD) is a major cause of chronic renal insufficiency. Without wishing to be bound by any particular theory, cyst expansion in PKD involves progressive fluid accumulation, which is believed to require chloride transport by the cystic fibrosis transmembrane conductance regulator (CFTR) protein. In vitro data implicates epithelial chloride secretion in generating and maintaining fluid-filled cysts (see, e.g., Ye et al., N. Engl. J. Med. 329:310-13 (1993); Davidow et al., Kidney Int. 50:208-18 (1996); Sullivan et al., Physiol. Rev. 78:1165-91 (1998); Li et al., Kidney Int. 66:1926-38 (2004)). CFTR, a cAMP-regulated chloride channel, is believed to provide the principal route for chloride entry into expanding cysts. CFTR is expressed in the apical membrane of cyst-lining epithelial cells in PKD kidneys (see, e.g., Sullivan et al., supra; Brill et al., Proc. Natl. Acad. Sci. USA 93:10206-211 (1996)). The CFTR inhibitor, CFTRᵢₙₕ-172 (see, e.g., Ma et al., J. Clin. Invest. 110:1651-48 (2002); U.S. Pat. No. 7,235,573), slows cyst growth in a MDCK cell culture model of PKD (Li et al., supra), and in metanephric kidney organ cultures (see, e.g., Magenheimer et al., J. Am. Soc. Nephrol. 17:3424-37 (2006)). In families affected with both ADPKD and cystic fibrosis, individuals with both ADPKD and cystic fibrosis had less severe renal disease than those with only ADPKD (see, e.g., O'Sullivan et al., Am. J. Kidney Dis. 32:976-83 (1998); Xu et al., J. Nephrol. 19:529-34 (2006)).

Without wishing to be bound by theory, CFTR inhibitors may block CFTR chloride channel function by different mechanisms. A thiazolidinone compound, CFTRᵢₙₕ-172 reversibly inhibits CFTR channel function (see, e.g., Ma et al., supra, 2002). Patch-clamp analysis indicated that CFTRᵢₙₕ-172 may stabilize the channel closed state by binding to a cytoplasmic domain of CFTR (see, e.g., Taddei et al., FEBS Lett. 558:52-56 (2004)). Following intravenous bolus infusion in rodents, CFTRᵢₙₕ-172 was concentrated in the kidney and urine with respect to blood, and was excreted with little metabolism (see, e.g., Sonawane et al., J Pharm. Sci. 94:134-143 (2004)). A glycine hydrazide CFTR inhibitor (such as GlyH-101 (see, e.g., US Patent Application Publication No. 2005/0239740) binds directly to the CFTR pore at a site near its external entrance (Muanprasat et al., supra, 2004). Provided herein are thiazolidinone derivative compounds and glycine hydrazide derivative compounds that have improved water solubility and that are useful for inhibiting cyst enlargement or cyst formation in a subject with PKD and for treating aberrantly increased intestinal fluid secretion.

### Thiazolidinone Derivative Compounds

Provided herein are thiazolidinone derivative compounds that are inhibitors of the cystic fibrosis transmembrane conductance regulator (CFTR) chloride channel. An embodiment provided herein is a thiazolidinone derivative compound, which has the following structure I:

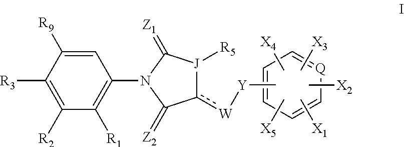

or a pharmaceutically acceptable salt, prodrug, or stereoisomer thereof, wherein

Y is —NH— or absent;

W is ═CH—, —S—, —O—, —C(═S)—, or —C(═O)—;

Z₁, Z₂, Z₃, Z₄, and Z₅ are each independently O or S;

J is C, S, O, or N;

Q is C or N;

R₁, R₂, R₃, and R₉ are each independently H, C₁₋₆ alkyl, alkoxy, halo, —CF₃, —CF₂CF₃, or —OCF₃;

R₅ is H, halo, C₁₋₆ alkyl, or absent;

X₁, X₂, X₃, and X₄ are each independently H, —OH, —SH, halo, tetrazolo, —P(═O)(OH)₂, —C(═Z₃)Z₄H, —Z₅—C(═Z₃)Z₄H, or —Z₅—CH₂—C(═Z₃)Z₄H; and

X₅ is —O⁻, tetrazolo, —C(═O)OH, —O—C(═O)OH, or absent.

In a particular embodiment, the compound of structure I has the substructure above wherein Q is N.

In other particular embodiments, Y is —NH— and W is —C(—S)— or —O—. In certain embodiments, Y is absent and W is —S— or —O—.

In another embodiment of the compound of structure I, Z₂ is O, Q is C, X₅ is absent, and the compound has the following structure I(A):

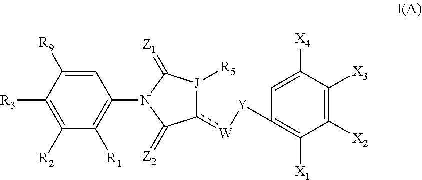

or a pharmaceutically acceptable salt, prodrug, or stereoisomer thereof, wherein

Y is —NH— or absent;

W is ═CH—, —S—, —O—, —C(═S)—, or —C(═O)—;

Z₁, Z₃, Z₄, and Z₅ are each independently O or S;

J is C, S, O, or N;

R₁, R₂, R₃, and R₉ are each independently H, C₁₋₆ alkyl, alkoxy, halo, —CF₃, —CF₂CF₃, or —OCF₃;

R₅ is H, halo, C₁₋₆ alkyl, or absent; and

X₁, X₂, X₃, and X₄ are each independently H, —OH, —SH, halo, tetrazolo, —P(═O)(OH)₂, —C(═Z₃)Z₄H, —Z₅—C(═Z₃)Z₄H, or —Z₅—CH₂—C(═Z₃)Z₄H.

The following are embodiments of a compound having the structure I or structure I(A).

In a specific embodiment of structure I and I(A), R₁, R₂, R₃, and R₉ are each independently H, C₁₋₆ alkyl, alkoxy, halo, —CF₃, —CF₂CF₃, or —OCF₃ and at least one of X₁, X₂, X₃, and X₄ is tetrazolo.

In certain embodiments, J is S and R₅ is absent.

In another specific embodiment, Y is —NH— and W is —C(═S)— or —C(═O)—. In yet another embodiment, Y is absent and W is —S— or —O—. In another specific embodiment of structure I and I(A), J is C, O, or N.

In another embodiment, at least one of R₁, R₂, R₃, and R₉ is —CH₃.

In other embodiments, at least one of X₁, X₂, X₃, and X₄ is tetrazolo or —Z₅—CH₂—C(═Z₃)Z₄H, wherein each of Z₃, Z₄, and Z₅ is independently O or S.

In yet other embodiments of structure I and I(A) and substructures described herein, at least one of R₁, R₂, R₃, and R₉ is —CF₃₅—CF₂CF₃, or —OCF₃. In another specific embodiment at least one of R₁, R₂, R₃, and R₉ is —CF₃ or —CH₃. In still another embodiment, at least one of one of R₁, R₂, R₃, and R₉ is —CF₃ and at least one of the remaining R₁, R₂, R₃, and R₉ is halo or —CH₃. In other specific embodiments, at least two of R₁, R₂, R₃, and R₉ are —CH₃. In a particular embodiment, R₂ is —CF₃, —CF₂CF₃, or —OCF₃.

In still another particular embodiment, at least one of R₁, R₂, R₃, and R₉ is —CF₂CF₃ or —OCF₃ and at least one of X₁, X₂, X₃, and X₄ is —C(═O)OH.

In a particular embodiment, J is S; R₅ is absent; Y is absent; W is ═CH—; R₁, R₂, R₃, and R₉ are each independently, H, halo or —CF₃ wherein at least two of R₁, R₂, R₃, and R₉ are H; and X₁, X₂, X₃, and X₄ are each independently H, —OH, —C(═O)OH, or —OCH₂C(═O)OH. In yet other certain embodiments, at least one of X₁, X₂, X₃, and X₄ is —OH. In a particular embodiment, J is S; R₅ is absent; Y is absent; W is ═CH—; and at least one of R₁, R₂, R₃, and R₉ is —CF₃ and the remaining of R₁, R₂, R₃, and R₉ are each independently halo, —CF₃, —CH₃, or H; and at least one of X₁, X₂, X₃, and X₄ is —OH and at least one of the remaining X₁, X₂, X₃, and X₄ is —C(═O)OH or —OCH₂C(═O)OH; or at least one of X₁, X₂, X₃, and X₄ is —OCH₂C(═O)OH; or at least 3 of X₁, X₂, X₃, and X₄ are not H. In a particular embodiment, at least three of X₁, X₂, X₃, and X₄ are —OH.

In another particular embodiment, J is S; R₅ is absent; Y is absent; W is ═CH—; at least two of R₁, R₂, R₃, and R₉ are not H; and X₁, X₂, X₃, and X₄ are each independently H, —OH, —C(═O)OH, or —OCH₂C(═O)OH. In other particular embodiments, at least one of R₁, R₂, R₃, and R₉ is —CF₃ and at least one of the remaining R₁, R₂, R₃, and R₉ is halo, which in certain embodiments is Cl or F.

In certain particular embodiments, for the compounds of structure of I and I(A), Z₁ is O.

In a particular embodiment, the compound of structure I and I(A) has a structure selected from:

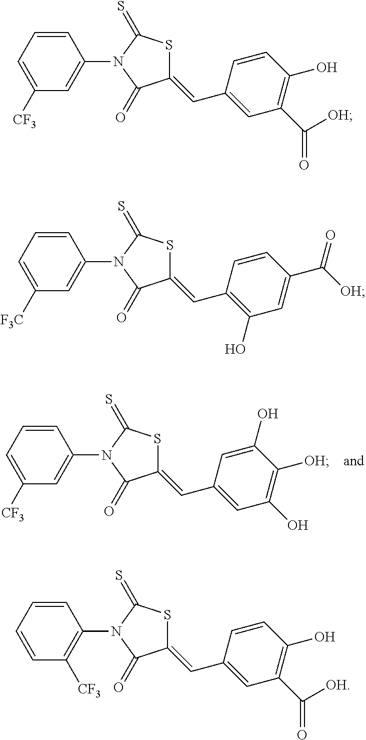

In other embodiments, the compound of structure I and I(A) has a structure selected from:

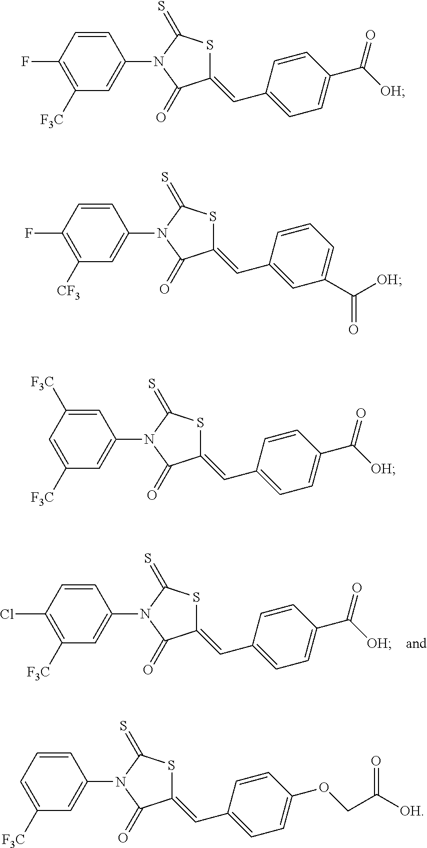

In one particular embodiment, the compound of structure I and I(A) has the following structure:

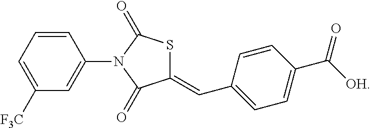

In another embodiment, a compound having the structure I(A) has a substructure wherein J is O and R₅ is absent, or J is C and R₅ is H, or J is N and R₅ is H or —CH₃; and X₃ is tetrazolo.

In other specific embodiments, the compounds of structure I and structure I(A) are described as above with the proviso that the following compounds are excluded from a compound having a structure I or I(A):

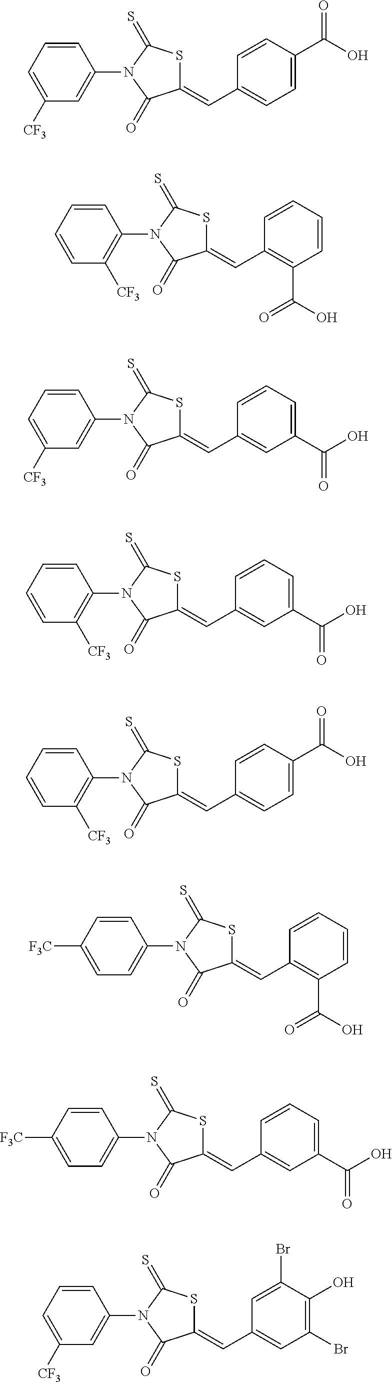

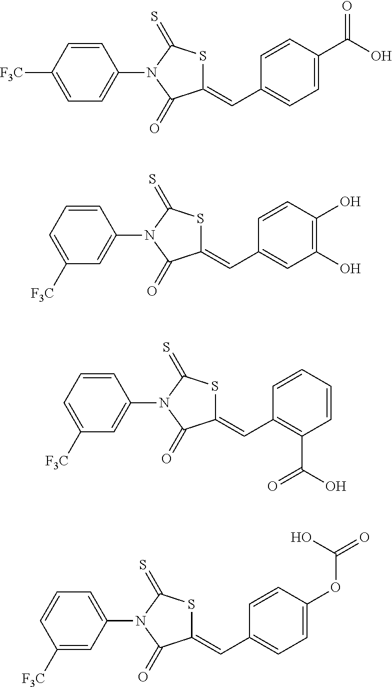

In another specific embodiment of structure I, Z₂ is O.

In an embodiment of structure I(A), J is S, R₅ is absent, and X₃ is tetrazolo-5-yl and the compound has the following structure I(A1):

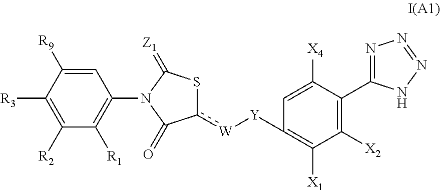

or a pharmaceutically acceptable salt, prodrug, or stereoisomer thereof, wherein

Y is —NH— or absent;

W is ═CH—, —S—, —O—, —C(═S)—, or —C(═O)—;

Z₁, Z₃, Z₄, and Z₅ are each independently O or S;

R₁, R₂, R₃, and R₉ are each independently H, C₁₋₆ alkyl, alkoxy, halo, —CF₃, —CF₂CF₃, or —OCF₃; and

X₁, X₂, and X₄ are each independently H, —OH, —SH, halo, —P(═O)(OH)₂, —C(═Z₃)Z₄H, —Z₅—C(═Z₃)Z₄H, or —Z₅—CH₂—C(═Z₃)Z₄H.

In another embodiment of the compound of structure I(A1), R₁, R₂, R₃, and R₉ are each independently H, —CH₃, halo, —CF₃, —CF₂CF₃, or —OCF₃. In a more specific embodiment, R₁, R₂, R₃, and R₉ are each independently H, —CH₃, chloro, fluoro, or —CF₃. In still other specific embodiments, at least one of R₁, R₂, R₃, and R₉ is —CH₃ or —CF₃. In still another embodiment, at least one of one of R₁, R₂, R₃, and R₉ is —CF₃ and at least one of the remaining R₁, R₂, R₃, and R₉ is halo or —CH₃. In yet other specific embodiments, at least two of R₁, R₂, R₃, and R₉ are —CH₃. In yet other specific embodiments, at least two of R₁, R₂, R₃, and R₉ are H. In a more specific embodiment, R₁ is —CF₃ or —CH₃; R₂ is —CF₃; and R₃ and R₉ are each H.

In yet another embodiment of the compound of structure I(A1), X₁, X₂, and X₄ are each independently H, —OH, bromo, —C(═O)OH, or —OCH₂C(═O)OH.

In another embodiment, a compound having the structure I(A) has a substructure in which wherein J is S, R₅ is absent, X₃ is tetrazolo-5-yl, Y is absent, each of X₁, X₂, and X₄ is H, and the compound has the following structure I(A2):

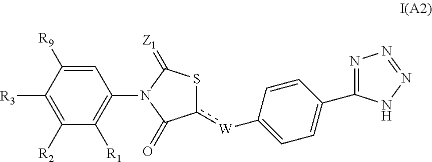

or a pharmaceutically acceptable salt, prodrug, or stereoisomer thereof, wherein

W is ═CH— or —S—;

Z₁ is O or S;

R₁, R₂, R₃, and R₉ are each independently H, C₁₋₆ alkyl, alkoxy, halo, —CF₃, —CF₂CF₃, or —OCF₃.

In a more specific embodiment, R₁, R₂, R₃, and R₉ are each independently H, —CH₃, chloro, fluoro, or —CF₃, —CF₂CF₃, or —OCF₃. In still other specific embodiments, at least one of R₁, R₂, R₃, and R₉ is —CH₃ or —CF₃. In other specific embodiments at least two of R₁, R₂, R₃, and R⁹ are H. In yet other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is halo or —CH₃. In yet other specific embodiments, at least two of R₁, R₂, R₃, and R₉ are H. In a more specific embodiment, R₁ is —CF₃ or —CH₃; R₂ is —CF₃; and R₃ and R₉ are each H.

In yet another embodiment, a compound having the structure I(A) has a substructure in which wherein J is S, R₅ is absent, X₃ is tetrazolo-5-yl, Z₁ is S, W is ═CH—, Y is absent, and the compound has the following structure I(A3):

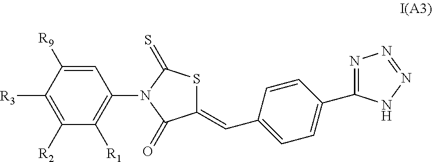

or a pharmaceutically acceptable salt, prodrug, or stereoisomer thereof, wherein R₁, R₂, R₃, and R₉ are each independently H, C₁₋₆ alkyl, —OCH₃, halo, —CF₃, —CF₂CF₃, or —OCF₃.

In a specific embodiment, R₁, R₂, R₃, and R₉ are each independently H, —CH₃, chloro, fluoro, or —CF₃. In a more specific embodiment, at least one of R₁, R₂, R₃, or R₉ is —CF₃ or —CH₃. In still another more specific embodiment, at least one of R₁, R₂, R₃, or R₉ is —CF₃. In yet another specific embodiment, at least one of R₁, R₂, R₃, or R₉ is —CH₃. In yet other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is halo or —CH₃. In other specific embodiments, at least two of R₁, R₂, R₃, or R₉ are H. In a more particular embodiment, R₁ is —CF₃ or —CH₃; R₂ is —CF₃; and R₃ and R₉ are each H.

In one specific embodiment, the compound of structure I and I(A), the compound has the following structure:

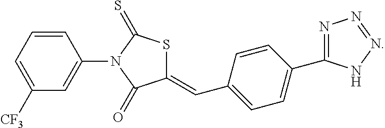

In another specific embodiment, the compound has a specific structure selected from:

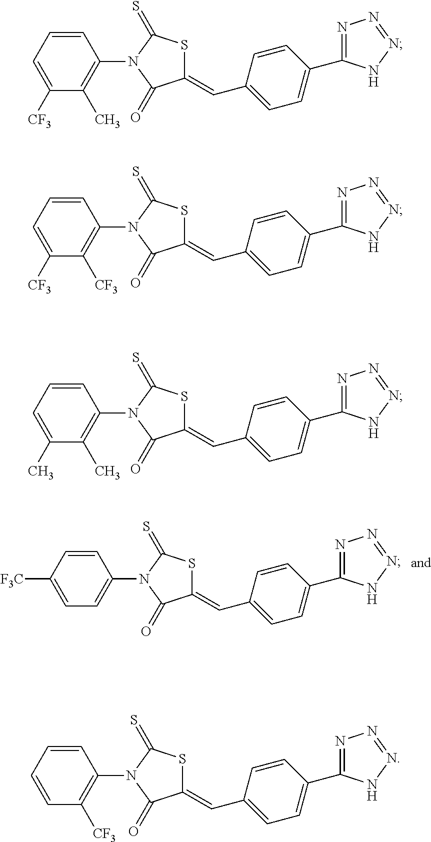

In another embodiment, the compound has a substructure of I(A) in which wherein J is S, R₅ is absent, X₃ is tetrazolo-5-yl, Z₁ is O, W is ═CH—, Y is absent, and the compound has the following structure I(A4):

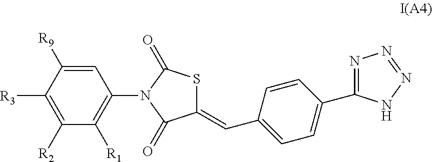

or a pharmaceutically acceptable salt, prodrug, or stereoisomer thereof, wherein R₁, R₂, R₃, and R₉ are each independently H, C₁₋₆ alkyl, —OCH₃, halo, —CF₃, —CF₂CF₃, or —OCF₃.

In a particular embodiment, R₁, R₂, R₃, and R₉ are each independently H, —CH₃, chloro, fluoro, or —CF₃. In a more specific embodiment, at least one of R₁, R₂, R₃, or R₉ is —CF₃ or —CH₃. In still another specific embodiment, at least one of R₁, R₂, R₃, or R₉ is —CF₃. In yet another specific embodiment, at least one of R₁, R₂, R₃, or R₉ is —CH₃. In yet other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is halo or —CH₃. In other specific embodiments, at least two of R₁, R₂, R₃, or R₉ are H. In a certain particular embodiment, R₁ is —CF₃ or —CH₃; R₂ is —CF₃; and R₃ and R₉ are each H.

In a more specific embodiment, the compound of structure I, I(A) and I(A4) has the following structure:

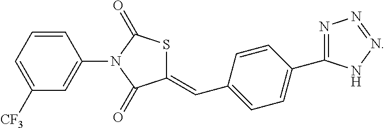

In another specific embodiment, the compound of structure I, I(A) and I(A4) the compound has a structure selected from:

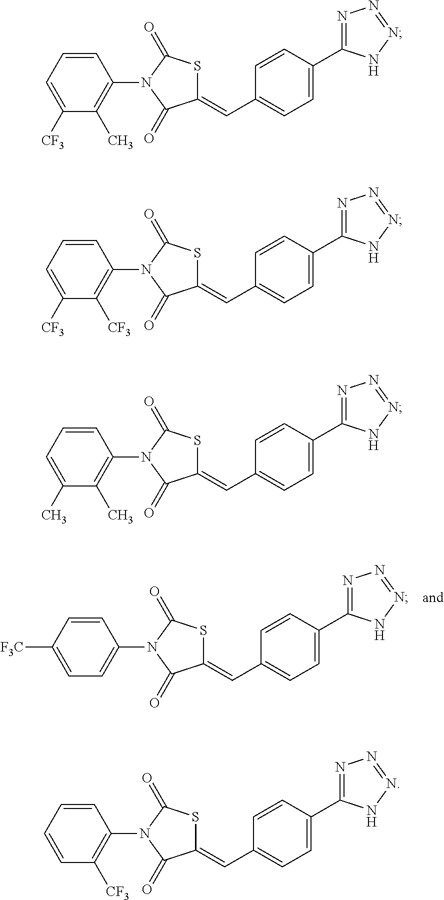

In another embodiment, a compound of structure I and I(A) has a substructure wherein J is S, R₅ is absent, Y is absent, and W is ═CH— and the compound has the following structure I(A5):

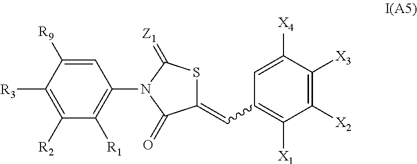

or a pharmaceutically acceptable salt, prodrug, or stereoisomer thereof, wherein

Z₁, Z₃, Z₄, and Z₅ are each independently O or S;

R₁, R₂, R₃, and R₉ are each independently H, C₁₋₆ alkyl, alkoxy, halo, —CF₃, —CF₂CF₃, or —OCF₃ wherein at least one of R₁, R₂, R₃, and R₉ is —CH₃; and

X₁, X₂, X₃, and X₄ are each independently H, —OH, —SH, halo, —P(═O)(OH)₂, —C(═Z₃)Z₄H, —Z₅—C(═Z₃)Z₄H, or —Z₅—CH₂—C(═Z₃)Z₄H.

In particular embodiments, Z₁ is S.

In other particular embodiments, each of X₁, X₂, and X₄ is H, and X₃ is either —C(═O)OH, —O—C(═O)OH, or —O—CH2-C(═O)OH. In a more specific embodiment, each of X₁, X₂, and X₄ is H, and X₃ is —C(═O)OH. In still another specific embodiment, X₁ and X₄ are each H, X₂ is —OH, and X₃ is —C(═O)OH; or X₁ and X₄ are each H, X₂ is —C(═O)OH, and X₃ is —OH; or X₁ is H or —OH, X₂ and X₄ are each bromo, and X₃ is —OH.

In another embodiment, at least one of R₁, R₂, R₃, and R₉ is —CF₃. In yet another embodiment, at least two of R₁, R₂, R₃, and R₉ are —CH₃. In yet another specific embodiment, a compound of structure I(A5) has a substructure wherein R₁ is either H or —CH₃; R₂ is —CH₃; and R₃ and R₉ are each H. In yet another specific embodiment, R₁ is —CH₃; R₂ is —CF₃; and R₃ and R₉ are each H. In still another specific embodiment, R₁ is —CH₃ and R₉ is —CF₃ or R₁ is —CF₃ and R₉ is —CH₃.

In specific embodiments, the compound of structure I, I(A) and I(A5) has a structure selected from:

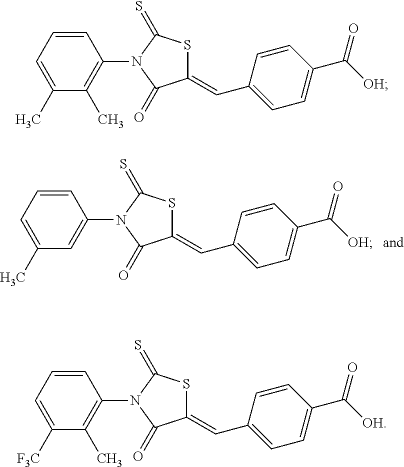

In other embodiments, a compound of structure I(A) has a substructure wherein J is S, R₅ is absent, Y is absent, and W is —S— and the compound has the following structure I(A6):

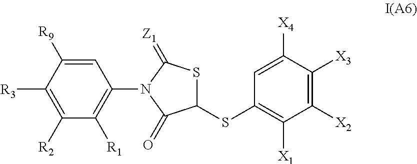

or a pharmaceutically acceptable salt, prodrug, or stereoisomer thereof, wherein

Z₁, Z₃, Z₄, and Z₅ are each independently O or S;

R₁, R₂, R₃, and R₉ are each independently H, C₁₋₆ alkyl, alkoxy, halo, —CF₃, —CF₂CF₃, or —OCF₃; and

X₁, X₂, X₃, and X₄ are each independently H, —OH, —SH, halo, —P(═O)(OH)₂, —C(═Z₃)Z₄H, —Z₅—C(═Z₃)Z₄H, or —Z₅—CH₂—C(═Z₃)Z₄H.

In a more specific embodiment, a compound of substructure I(A6) is provided wherein Z₁ is O.

In certain embodiments, each of R₁, R₂, R₃, and R₉ is independently H, —CH₃, chloro, fluoro, or —CF₃. In other specific embodiments, at least one of R₁, R₂, R₃, and R₉ is —CF₃ or —CH₃. In yet other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is halo or —CH₃. In a specific embodiment, at least two or at least three of R₁, R₂, R₃, and R₉ is H. In yet other specific embodiments, R₁, R₃, and R₉ are each H and R₂ is —CF₃.

In certain embodiments, a compound of structure I(A6) is provided wherein at least one of X₁, X₂, X₃, and X₄ is —C(═O)OH. In a particular embodiment, each of X₁, X₂, and X₄ is H, and X₃ is —C(═O)OH.

In a more specific embodiment, the compound of structure I(A) and substructure I(A6) has the following structure:

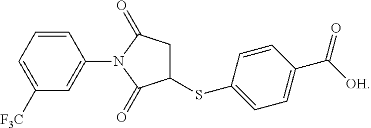

In yet another embodiment, a compound of structure I(A) is provided wherein J is S, R₅ is absent, Y is —NH— and W is —C(═S)— and the compound has the following structure I(A7):

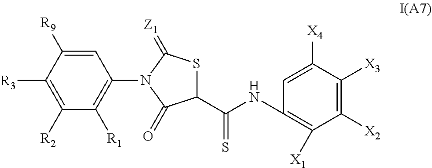

or a pharmaceutically acceptable salt, prodrug, or stereoisomer thereof, wherein

Z₁, Z₃, Z₄, and Z₅ are each independently O or S;

R₁, R₂, R₃, and R₉ are each independently H, C₁₋₆ alkyl, alkoxy, halo, —CF₃, —CF₂CF₃, or —OCF₃; and

X₁, X₂, X₃, and X₄ are each independently H, —OH, —SH, halo, —P(═O)(OH)₂, —C(═Z₃)Z₄H, —Z₅—C(═Z₃)Z₄H, or —Z₅—CH₂—C(═Z₃)Z₄H.

In a specific embodiment, a compound of structure I(A7) is provided wherein Z₁ is S.

In another specific embodiment, each of each of R₁, R₂, R₃, and R₉ is independently H, —CH₃, chloro, fluoro, or —CF₃. In a more specific embodiment, at least one of R₁, R₂, R₃, and R₉ is —CF₃ or —CH₃. In yet other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is halo or —CH₃. In a specific embodiment, at least two or at least three of R₁, R₂, R₃, and R₉ are H. In yet another specific embodiment, R₁, R₃, and R₉ are each H and R₂ is —CF₃.

In another embodiment, a compound of structure I(A7) is provided wherein at least one of X₁, X₂, X₃, and X₄ is —C(═O)OH. In a particular embodiment, each of X₁, X₂, and X₄ is H, and X₃ is —C(═O)OH.

In a specific embodiment the compound of structure I(A) and substructure I(A7) has a structure selected from:

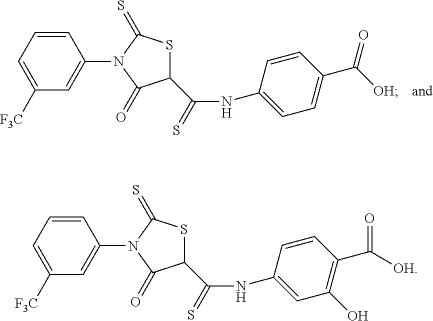

In yet another embodiment, a compound of structure I(A) is provided wherein J is S, R₅ is absent, Y is absent, W is ═CH—, each of X₁ and X₃ is —OH, and each of X₂ and X₄ is Br, wherein the compound has the following structure I(A8):

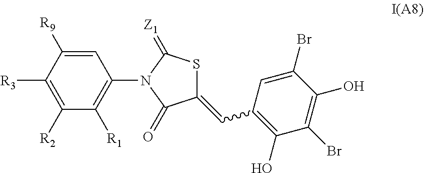

or a pharmaceutically acceptable salt, prodrug, or stereoisomer thereof,

wherein Z₁ is O or S; and

each of R₁, R₂, R₃, and R₉ is independently H, C₁₋₆ alkyl, alkoxy, halo, —CF₃, —CF₂CF₃, or —OCF₃.

In a specific embodiment of the compound of substructure I(A8), each of R₁, R₂, R₃, and R₉ is independently H, —CH₃, chloro, fluoro, or —CF₃. In yet another specific embodiment, at least one of R₁, R₂, R₃, and R₉ is —CF₃ or —CH₃. In more specific embodiments, at least two of R₁, R₂, R₃, and R₉ are H. In still a more specific embodiment, R₂ is —CF₃.

In other embodiments, a compound of substructure I(A8) is provided wherein Z₁ is S.

In a more specific embodiment a compound of structure I(A) and substructure I(A8) has the following structure:

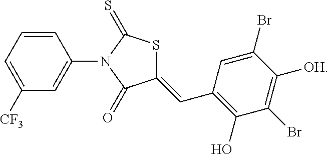

In another embodiment, a compound of structure I is provided, wherein Q is N, X₅ is absent, Z₂ is O, J is S and R₅ is absent, and the compound has the following structure I(B):

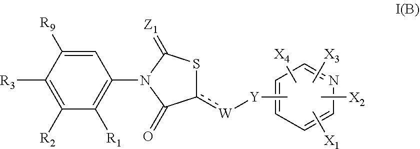

or a pharmaceutically acceptable salt, prodrug, or stereoisomer thereof, wherein

Y is —NH— or absent;

W is ═CH—, —S—, —O—, —C(═S)—, or —C(═O)—;

Z₁, Z₃, Z₄, and Z₅ are each independently O or S;

R₁, R₂, R₃, and R₉ are each independently H, C₁₋₆ alkyl, alkoxy, halo, —CF₃, —CF₂CF₃, or —OCF₃; and

X₁, X₂, X₃, and X₄ are each independently H, —OH, —SH, halo, tetrazolo, —P(═O)(OH)₂, —C(═Z₃)Z₄H, —Z₅—C(═Z₃)Z₄H, or —Z₅—CH₂—C(═Z₃)Z₄H.

In a more specific embodiment, Y is —NH— or absent, and W is ═CH—, —S—, or —C(═S)—. In another specific embodiment, Y is absent and W is ═CH—.

In another embodiment, a compound of structure I and I(B) is provided, wherein X₁, X₂, X₃, and X₄ are each independently H, —OH, halo, or tetrazolo, —C(═O)OH, —O—C(═O)OH, or —O—CH₂—C(═O)OH.

In another specific embodiment, R₁, R₂, R₃, and R₉ are each independently H, —CH₃, halo, —CF₃, —CF₂CF₃, or —OCF₃. In still another embodiment, at least one of R₁, R₂, R₃, and R₉ is —CH₃ or —CF₃. In yet other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is halo or —CH₃. In particular embodiments, at least two of R₁, R₂, R₃, and R₉ are H. In still another specific embodiment, R₂ is —CF₃.

In yet another specific embodiment of the compound of structure I(B), each of each of X₁, X₂, X₃, and X₄ is H, Y is absent, and W is ═CH— and the compound has the following structure I(B1):

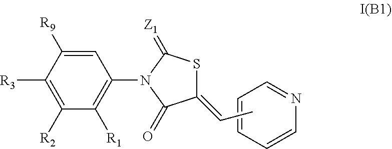

wherein

Z₁ is O or S; and

R₁, R₂, R₃, and R₉ are each independently H, —CH₃, halo, —CF₃, —CF₂CF₃, or —OCF₃.

In other specific embodiments of the compound of structure I(B) and I(B1), at least one of R₁, R₂, R₃, and R₉ is —CF₃ or —CH₃. In a more specific embodiment, R₂ is —CF₃. In other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is —CH₃. In particular embodiments, at least two of R₁, R₂, R₃, and R₉ are H.

Also provided is a compound of structure I(B) and I(B1) wherein Z₁ is S.

In a particular embodiment, a compound of structure I(B) and I(B1) has the following structure:

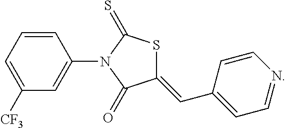

In another embodiment, a compound of structure I is provided, wherein Q is N, Z₂ is O, J is S, R₅ is absent, and the compound has the following structure I(C):

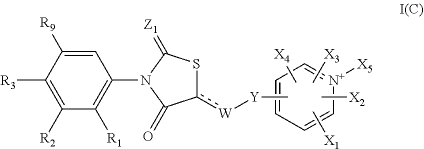

or a pharmaceutically acceptable salt, prodrug, or stereoisomer thereof, wherein

Y is —NH— or absent;

W is ═CH—, —S—, —O—, —C(═S)—, or —C(═O)—;

Z₁, Z₃, Z₄, and Z₅ are each independently O or S;

R₁, R₂, R₃, and R₉ are each independently H, C₁₋₆ alkyl, alkoxy, halo, —CF₃, —CF₂CF₃, or —OCF₃;

X₁, X₂, X₃, and X₄ are each independently H, —OH, —SH, halo, —P(═O)(OH)₂, —C(═Z₃)Z₄H, —Z₅—C(═Z₃)Z₄H, or —Z₅—CH₂—C(═Z₃)Z₄H; and

X₅ is —O⁻, tetrazolo, —C(═O)OH, or —O—C(═O)OH.

In a specific embodiment, a compound of structure I and I(C) is provided, wherein Y is —NH— or absent, and W is ═CH—, —S—, or —C(═S)—.

In another particular embodiment, X₁, X₂, X₃, and X₄ are each independently H, —OH, halo, —C(═O)OH, —O—C(═O)OH, or —O—CH₂—C(═O)OH.

In still another certain embodiment, R₁, R₂, R₃, and R₉ are each independently H, —CH₃, halo, —CF₃, —CF₂CF₃, or —OCF₃. In still another embodiment, at least one of R₁, R₂, R₃, and R₉ is —CH₃ or —CF₃. In yet other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is —CH₃. In another specific embodiment at least two of R₁, R₂, R₃, and R₉ are H. In a certain embodiment, R₂ is —CF₃.

In a specific embodiment, a compound of structure I and I(C) is provided wherein each of each of X₁, X₂, X₃, and X₄ is H, Y is absent, and W is ═CH— and the compound has the following structure I(C1):

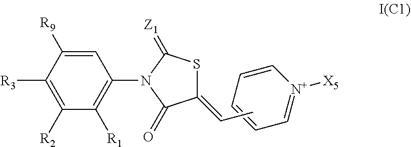

or a pharmaceutically acceptable salt, prodrug, or stereoisomer thereof, wherein

Z₁ is O or S;

R₁, R₂, R₃, and R₉ are each independently H, —CH₃, halo, —CF₃, —CF₂CF₃, or —OCF₃; and

X₅ is —O⁻, tetrazolo, —C(═O)OH, or —O—C(═O)OH.

In a more specific embodiment of structure I(C1), at least one of R₁, R₂, R₃, and R₉ is —CF₃ or —CH₃. In yet other specific embodiments, at least two of R₁, R₂, R₃, and R₉ are —CH₃. In still another embodiment, at least one of one of R₁, R₂, R₃, and R₉ is —CF₃ and at least one of the remaining R₁, R₂, R₃, and R₉ is —CH₃. In another specific embodiment at least two of R₁, R₂, R₃, and R₉ are H. In a certain embodiment, R₂ is —CF₃.

In one embodiment of the compound of structure I(C1), Z₁ is S.

In a specific embodiment the compound of substructure I(C1) has the following structure:

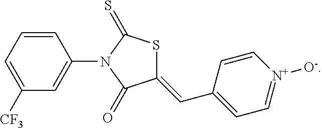

### Glycine Hydrazide Derivative Compounds

Provided herein are glycine hydrazide derivative compounds that are inhibitors of the cystic fibrosis transmembrane conductance regulator (CFTR) chloride channel. An embodiment provided herein is a glycine hydrazide derivative compound, which has the following structure II:

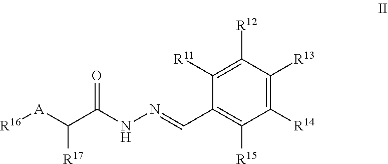

or a pharmaceutically acceptable salt, prodrug, or stereoisomer thereof, wherein

A is —O— or —NH—;

R¹¹, R¹², R¹³, R¹⁴, R¹⁵ are each the same or different and independently hydrogen, hydroxy, C₁₋₆ alkyl, C₁₋₆ alkoxy, carboxy, halo, nitro, aryl, and heteroaryl;

R¹⁶ is phenyl, heteroaryl, quinolinyl, anthracenyl, or naphthalenyl; and

R¹⁷ is H, alkoxy, or substituted or unsubstituted aryl.

In a particular embodiment, when A is —NH—, R¹⁷ is unsubstituted phenyl or substituted phenyl wherein phenyl is substituted with halo, C₁₋₆ alkyl, C₁₋₆ alkoxy, or carboxy. In another particular embodiment, when A is —O—, R¹⁷ is H.

Also provided herein is a compound of structure II, wherein A is —O— and R¹⁷ is H, and the compound has the following structure II(A):

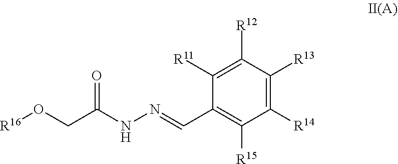

or a pharmaceutically acceptable salt, prodrug, or stereoisomer thereof, wherein

R¹⁶ is phenyl, heteroaryl, quinolinyl, anthracenyl, or naphthalenyl; and

R¹¹, R¹², R¹³, R¹⁴, R¹⁵ are each the same or different and independently hydrogen, hydroxy, C₁₋₆ alkyl, C₁₋₆ alkoxy, carboxy, halo, nitro, aryl, and heteroaryl.

In certain embodiments, R¹⁶ is phenyl, heteroaryl, quinolinyl, or anthracenyl. In a more specific embodiment, R¹⁶ is unsubstituted phenyl, or substituted phenyl wherein phenyl is substituted with one or more of hydroxy, methyl, or halo. In a particular embodiment, halo is chloro or fluoro. In other specific embodiments, R¹⁶ is 2-naphthalenyl, 1-naphthalenyl, 2-chlorophenyl, 4-chlorophenyl, 2,4-chlorophenyl, 4-methylphenyl, 2-anthracenyl, 7-quinolinyl, or 6-quinolinyl. In more particular embodiments, R¹⁶ is 2-chlorophenyl, 4-chlorophenyl, or 2,4-chlorophenyl.

In still other embodiments, R¹¹, R¹², R¹³, R¹⁴, R¹⁵ are each the same or different and independently hydrogen, hydroxy, carboxy, or halo. In more specific embodiments, R¹¹ is H, each of R¹² and R¹⁴ is halo and each of R¹³ and R¹⁵ is hydroxy. In another specific embodiment, R¹¹ is H, each of R¹² and R¹⁴ is halo, R¹³ is hydroxyl, and R¹⁵ is H. In certain particular embodiments, halo is bromo. In another particular embodiment, R¹¹ is H, each of R¹² and R¹⁴ is bromo, and each of R¹³ and R¹⁵ is hydroxyl. In still another specific embodiment, R¹¹ is H, each of R¹² and R¹⁴ is bromo, R¹³ is hydroxy, and R¹⁵ is hydrogen.

In particular embodiments, the compound having a structure of II and II(A) has a structure selected from:

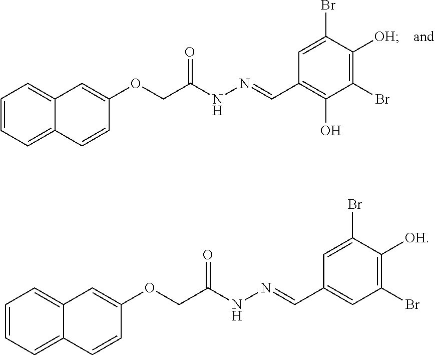

As set forth above, in one embodiment, a glycine hydrazide derivative compound has the following structure II:

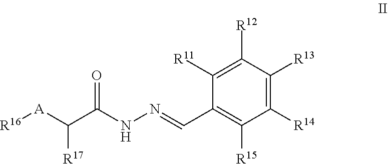

or a pharmaceutically acceptable salt, prodrug, or stereoisomer thereof, wherein

A is —O— or —NH—;

R¹¹, R¹², R¹³, R¹⁴, R¹⁵ are each the same or different and independently hydrogen, hydroxy, C₁₋₆ alkyl, C₁₋₆ alkoxy, carboxy, halo, nitro, aryl, and heteroaryl;

R¹⁶ is phenyl, heteroaryl, quinolinyl, anthracenyl, or naphthalenyl; and

R¹⁷ is H, alkoxy, or substituted or unsubstituted aryl,

wherein in certain embodiments, A is —NH—, R¹⁷ is unsubstituted phenyl or substituted phenyl wherein phenyl is substituted with halo, C₁₋₆ alkyl, C₁₋₆ alkoxy, or carboxy.

In certain embodiments, R¹⁶ is phenyl, heteroaryl, quinolinyl, or anthracenyl, or 2-naphthalenyl. In other certain embodiments, R¹⁶ is phenyl, heteroaryl, quinolinyl, or anthracenyl. In a specific embodiment, a compound has the structure II or II(A) wherein when A is —O— and R¹⁷ is H, R¹¹ is H, R¹² is Br, R¹³ is OH, R¹⁴ is Br, and R¹⁵ is H, then R¹⁶ is not 1-naphthalenyl.

In other embodiments, a compound of structure II is provided wherein A is —NH— and R¹⁷ is unsubstituted phenyl, and the compound has the following structure II(B):

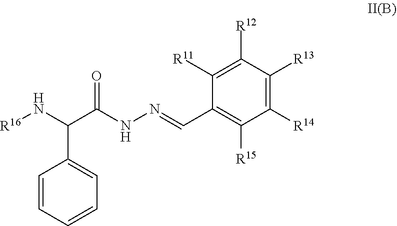

or a pharmaceutically acceptable salt, prodrug, or stereoisomer thereof, wherein

R¹⁶ is phenyl, heteroaryl, quinolinyl, anthracenyl, or naphthalenyl; and

R¹¹, R¹², R¹³, R¹⁴, R¹⁵ are each the same or different and independently hydrogen, hydroxy, C₁₋₆ alkyl, C₁₋₆ alkoxy, carboxy, halo, nitro, aryl, and heteroaryl.

In particular embodiments, R¹⁶ is unsubstituted phenyl, or substituted phenyl wherein phenyl is substituted with one or more of hydroxy, methyl, or halo. In more specific embodiments, halo is chloro or fluoro. In yet other embodiments, R¹⁶ is 2-naphthalenyl, 1-naphthalenyl, 2-chlorophenyl, 4-chlorophenyl, 2,4-chlorophenyl, 4-methylphenyl, 2-anthracenyl, 7-quinolinyl, or 6-quinolinyl. In certain specific embodiments, R¹⁶ is 2-chlorophenyl, 4-chlorophenyl, or 2,4-chlorophenyl.

In other particular embodiments, a compound of structure II and II(B) is provided wherein R¹¹, R¹², R¹³, R¹⁴, R¹⁵ are each the same or different and independently hydrogen, hydroxy, carboxy, or halo. In a more specific embodiment, R¹¹ is H, each of R¹² and R¹⁴ is halo and each of R¹³ and R¹⁵ is hydroxy. In still another specific embodiment, R¹¹ is H, each of R¹² and R¹⁴ is halo, R¹³ is hydroxyl, and R¹⁵ is H. In certain specific embodiments, halo is bromo. In yet another embodiment, R¹¹ is H, each of R¹² and R¹⁴ is bromo, and each of R¹³ and R¹⁵ is hydroxy. In other embodiments, R¹¹ is H, each of R¹² and R¹⁴ is bromo, R¹³ is hydroxy, and R¹⁵ is hydrogen.

In specific embodiments, a compound of structure II and II(B) has either of the following structures:

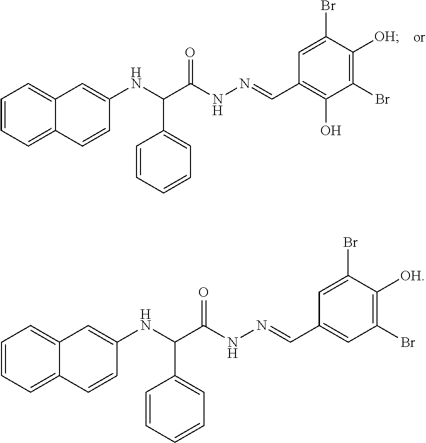

Pharmaceutical Compositions and Methods of Using the Compounds

As described in greater detail herein, in other embodiments, pharmaceutical compositions are provided wherein the pharmaceutical composition comprises a pharmaceutically suitable excipient and at least one of the compounds of any one of the structures, substructures, and specific compounds described herein, including a compound of structure I and substructures I(A), I(A1-A8), I(B), I(B1), I(C), I(C1) and specific structures as described herein (i.e., thiazolidinone derivative compounds) and/or at least one compound of structure II and substructures II(A) and II(B) and specific structures described herein (i.e., glycine hydrazide derivative compounds).

In other embodiments, a method is provided for inhibiting cyst formation or cyst enlargement comprising contacting (a) a cell that comprises CFTR and (b) at least one compound of structure I and substructures I(A), I(A1-A8), I(B), I(B1), I(C), I(C1) and specific structures as described herein (i.e., thiazolidinone derivative compounds) and/or at least one compound of structure II and substructures II(A) and II(B) and specific structures described herein (i.e., glycine hydrazide derivative compounds), under conditions and for a time sufficient for the CFTR and the compound to interact, wherein the compound inhibits ion transport by CFTR. In particular embodiments, the cyst formation or cyst enlargement that is inhibited is kidney cyst formation or kidney cyst enlargement (i.e., cyst formation or enlargement in at least one kidney is inhibited.)

In yet another embodiment, a method is provided for treating polycystic kidney disease comprising administering to subject a (a) pharmaceutically suitable excipient and (b) at least one of the compounds of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein (i.e., a pharmaceutical composition as described herein). In a specific embodiment, polycystic kidney disease is autosomal dominant polycystic kidney disease. In another specific embodiment, polycystic kidney disease is autosomal recessive polycystic kidney disease.

In another embodiment, a method is provided for treating a disease or disorder associated with aberrantly increased ion transport by cystic fibrosis transmembrane conductance regulator (CFTR), the method comprising administering to a subject a pharmaceutically suitable excipient and at least one of the compounds of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein (i.e., a pharmaceutical composition as described herein), wherein ion transport by CFTR is inhibited. In a specific embodiment, the disease or disorder is aberrantly increased intestinal fluid secretion. In a more specific embodiment, the disease or disorder is secretory diarrhea. In a specific embodiment, secretory diarrhea is caused by an enteric pathogen. In particular embodiments, the enteric pathogen is Vibrio cholerae, Clostridium difficile, Escherichia coli, Shigella, Salmonella, rotavirus, Giardia lamblia, Entamoeba histolytica, Campylobacter jejuni, and Cryptosporidium. In other specific embodiments, the secretory diarrhea is induced by an enterotoxin. In particular embodiments, the enterotoxin is a cholera toxin, a E. coli toxin, a Salmonella toxin, a Campylobacter toxin, or a Shigella toxin. In other particular embodiments, secretory diarrhea is a sequelae of ulcerative colitis, irritable bowel syndrome (IBS), AIDS, chemotherapy, or an enteropathogenic infection.

In particular embodiments of the methods described herein, the subject is a human or non-human animal.

In another embodiment, a method is provided for inhibiting ion transport by a cystic fibrosis transmembrane conductance regulator (CFTR) comprising contacting (a) a cell that comprises CFTR and (b) at least one of the compounds of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein, under conditions and for a time sufficient that permit the CFTR and the compound to interact, thereby inhibiting ion transport (e.g., chloride ion transport) by CFTR.

In another embodiment, a method is provided for treating secretory diarrhea comprising administering to a subject a pharmaceutically acceptable excipient and at least one of the compounds of structure I, substructures I(A), I(A1-A8), I(B), I(B1), I(C), I(C1) (i.e., thiazolidinone derivative compounds) and specific structures as described herein and/or at least one of the compounds of structure II and substructures II(A) and II(B) (i.e., glycine hydrazide derivative compounds) and specific structures described herein (i.e., a pharmaceutical composition as described herein). In a particular embodiment, the subject is a human or non-human animal.

In particular embodiments of each of the methods described in detail herein, (including the method of inhibiting cyst formation or cyst enlargement, the method of treating polycystic kidney disease, the method of treating a disease or disorder associated with aberrantly increased ion transport by cystic fibrosis transmembrane conductance regulator (CFTR), method of inhibiting ion transport by CFTR, and the method of treating secretory diarrhea), the compound is selected from:

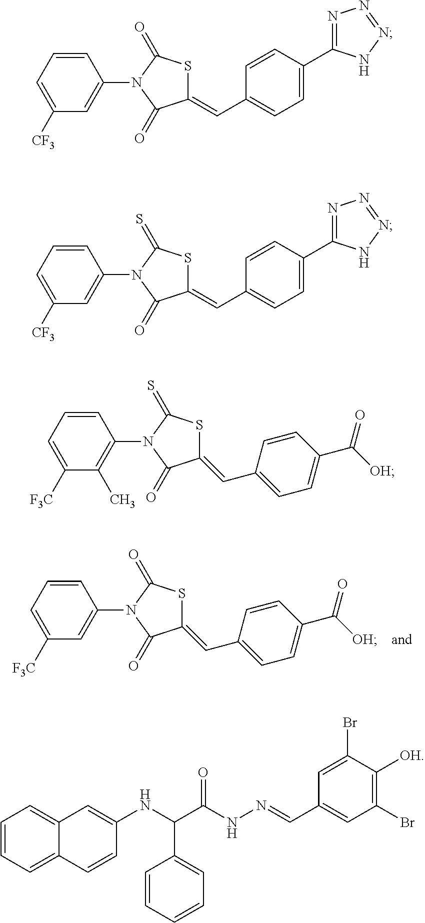

In a specific embodiment, a method is provided for inhibiting cyst formation or cyst enlargement comprising contacting (a) a cell that comprises CFTR and (b) a compound that inhibits ion transport by CFTR, under conditions and for a time sufficient that permit CFTR and the compound to interact, wherein the compound has the following structure:

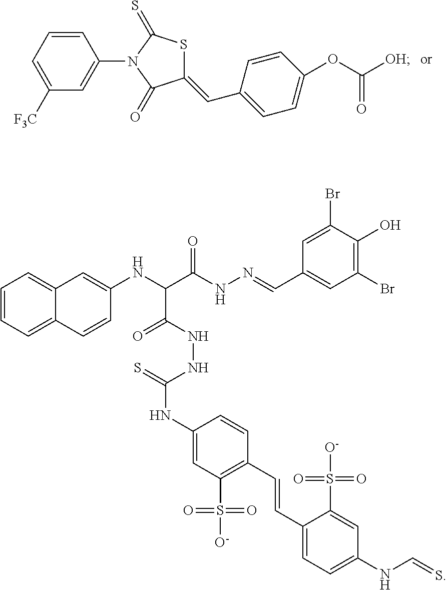

In another embodiment, a method is provided for treating polycystic kidney disease comprising administering to subject a pharmaceutical composition that comprises a pharmaceutically suitable excipient and a compound having a structure:

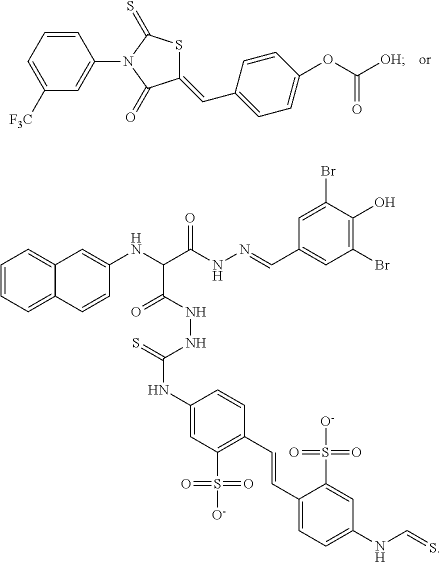

In a particular embodiment, polycystic kidney disease is autosomal dominant polycystic kidney disease. In another particular embodiment, polycystic kidney disease is autosomal recessive polycystic kidney disease.

### Chemistry Definitions

Certain chemical groups named herein are preceded by a shorthand notation indicating the total number of carbon atoms that are to be found in the indicated chemical group. For example; C₁-C₆ alkyl describes an alkyl group, as defined below, having a total of 1 to 6 carbon atoms, and C₃-C₁₂ cycloalkyl describes a cycloalkyl group, as defined below, having a total of 3 to 12 carbon atoms. The total number of carbons in the shorthand notation does not include carbons that may exist in substituents of the group described. In addition to the foregoing, as used herein, unless specified to the contrary, the following terms have the meaning indicated.

“Alkyl” means a straight chain or branched, noncyclic or cyclic, unsaturated or saturated aliphatic hydrocarbon containing from 1 to 18 carbon atoms, while the term “C₁₋₆ alkyl” has the same meaning as alkyl but contain from 1 to 6 carbon atoms. Representative saturated straight chain alkyls include methyl, ethyl, n-propyl, n-butyl, n-pentyl, n-hexyl, and the like, while saturated branched alkyls include isopropyl, sec-butyl, isobutyl, tert-butyl, heptyl, n-octyl, isopentyl, 2-ethylhexyl and the like. Representative saturated cyclic alkyls include cyclopropyl, cyclobutyl, cyclopentyl, cyclohexyl, —CH₂cyclopropyl, —CH₂cyclobutyl, —CH₂cyclopentyl, —CH₂cyclohexyl, and the like; unsaturated cyclic alkyls include cyclopentenyl and cyclohexenyl, and the like. Cyclic alkyls, also referred to as “homocyclic rings,” include di- and poly-homocyclic rings such as decalin and adamantyl. Unsaturated alkyls contain at least one double or triple bond between adjacent carbon atoms (referred to as an “alkenyl” or “alkynyl,” respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; representative straight chain and branched alkynyls include acetylenyl, propynyl, 1-butynyl, 2-butynyl, 1-pentynyl, 2-pentynyl, 3-methyl-1 butynyl, and the like.

It is understood that within the context of the compounds described herein that the terms alkyl, aryl, heteroaryl, arylalkyl, heterocycle, homocycle, and heterocycloalkyl are taken to comprise unsubstituted alkyl and substituted alkyl, unsubstituted aryl and substituted aryl, unsubstituted heteroaryl and substituted heteroaryl, unsubstituted arylalkyl and substituted arylalkyl, unsubstituted heterocycle and substituted heterocycle, unsubstituted homocycle and substituted homocycle, unsubstituted heterocycloalkyl and substituted heterocyclealkyl, respectively, as defined herein, unless otherwise specified.

As used herein, the term “substituted” in the context of alkyl, aryl, arylalkyl, heterocycle, heteroaryl, and heterocycloalkyl means that at least one hydrogen atom of the alky, aryl, arylalkyl, heterocycle, heteroaryl, or heterocycloalkyl moiety is replaced with a substituent. In the instance of an oxo substituent (“═O”) two hydrogen atoms are replaced. A “substituent” as used within the context of this disclosure includes oxo, halogen, hydroxy, cyano, nitro, amino, alkylamino, dialkylamino, alkyl, alkoxy, thioalkyl, haloalkyl, substituted alkyl, heteroalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocycloalkyl, substituted heterocycloalkyl, —NRₐR_{b}, —NRₐC(═O)R_{b}, —NRₐC(═O)NRₐR_{b}, —NRₐC(═O)OR_{b} —NRₐS(═O)₂R_{b}, —ORₐ, —C(═O)Rₐ —C(═O)ORₐ, —C(═O)NRₐR_{b}, —OCH₂C(═O)NRₐR_{b}, —OC(═O)NRₐR_{b}, —SH, —SRₐ, —SORₐ, —S(═O)₂NRₐR_{b}, —S(═O)₂Rₐ, —SRₐC(═O)NRₐR_{b}, —OS(═O)₂Rₐ and —S(═O)₂ORₐ (also written as —SO₃Rₐ), wherein Rₐ and R_{b} are the same or different and independently hydrogen, alkyl, haloalkyl, substituted alkyl, alkoxy, aryl, substituted aryl, arylalkyl, substituted arylalkyl, arylalkoxy, heteroaryl, substituted heteroaryl, heteroarylalkyl, substituted heteroarylalkyl, heterocycle, substituted heterocycle, heterocycloalkyl or substituted heterocycloalkyl. The definitions of Rₐ and R_{b} above apply to all uses of these substituents throughout the description.

Representative substituents include (but are not limited to) alkoxy (i.e., alkyl-O—, including C₁₋₆ alkoxy e.g., methoxy, ethoxy, propoxy, butoxy, pentoxy,), aryloxy (e.g., phenoxy, chlorophenoxy, tolyloxy, methoxyphenoxy, benzyloxy, alkyloxycarbonylphenoxy, alkyloxycarbonyloxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkyloxycarbonyl-phenylthio), amino (e.g., amino, mono- and di-C₁-C₃ alkanylamino, methylphenylamino, methylbenzylamino, C₁-C₃ alkanylamido, acylamino, carbamamido, ureido, guanidino, nitro and cyano). Moreover, any substituent may have from 1-5 further substituents attached thereto.

“Aryl” means an aromatic carbocyclic moiety such as phenyl or naphthyl (i.e., naphthalenyl) (1- or 2-naphthyl) or anthracenyl (e.g., 2-anthracenyl).

“Arylalkyl” (e.g., phenylalkyl) means an alkyl having at least one alkyl hydrogen atom replaced with an aryl moiety, such as —CH₂-phenyl, —CH═CH-phenyl, —C(CH₃)═CH-phenyl, and the like.

“Heteroaryl” means an aromatic heterocycle ring of 5- to 10 members and having at least one heteroatom selected from nitrogen, oxygen, and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl (including 6-quinolinyl and 7-quinolinyl), isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined herein. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “optionally substituted” as used in the context of an optionally substituted heterocycle (as well heteroaryl) means that at least one hydrogen atom is replaced with a substituent. In the case of a keto substituent (“—C(═O)—”) two hydrogen atoms are replaced. When substituted, one or more of the above groups are substituted. “Substituents” within the context of description herein are also described above and include halogen, hydroxy, cyano, nitro, amino, alkylamino, dialkylamino, alkyl, alkoxy, alkylthio, haloalkyl, aryl, arylalkyl, heteroaryl, heteroarylalkyl, heterocycle and heterocycloalkyl, as well as —NRₐR_{b}, —NRₐC(═O)R_{b}, —NRₐC(═O)NRₐR_{b}, —NRₐC(═O)OR_{b}—NRₐS(═O)₂R_{b}, —ORₐ, —C(═O)Rₐ —C(═O)ORₐ, —C(═O)NRₐR_{b}, —OCH₂C(═O)NRₐR_{b}, —OC(═O)NRₐR_{b}, —SH, —SRₐ, —SORₐ, —S(═O)₂NRₐR_{b}, —S(═O)₂Rₐ, —OS(═O)₂Rₐ and —S(═O)₂ORₐ. In addition, the above substituents may be further substituted with one or more of the above substituents, such that the substituent is a substituted alkyl, substituted aryl, substituted arylalkyl, substituted heterocycle or substituted heterocycloalkyl. Rₐ and R_{b} in this context may be the same or different and independently hydrogen, alkyl, haloalkyl, substituted alkyl, alkoxy, aryl, substituted aryl, arylalkyl, substituted arylalkyl, heterocycle (including heteroaryl), substituted heterocycle (including substituted heteroaryl), heterocycloalkyl, or substituted heterocycloalkyl.

“Heterocycloalkyl” means an alkyl having at least one alkyl hydrogen atom replaced with a heterocycle, such as —CH₂morpholinyl, —CH₂CH₂piperidinyl, —CH₂azepineyl, —CH₂pirazineyl, —CH₂pyranyl, —CH₂furanyl, —CH₂pyrrolidinyl, and the like.

“Homocycle” (also referred to herein as “homocyclic ring”) means a saturated or unsaturated (but not aromatic) carbocyclic ring containing from 3-7 carbon atoms, such as cyclopropane, cyclobutane, cyclopentane, cyclohexane, cycloheptane, cyclohexene, and the like.

“Halogen” or “halo” means fluoro, chloro, bromo, and iodo.

“Haloalkyl,” which is an example of a substituted alkyl, means an alkyl having at least one hydrogen atom replaced with halogen, such as trifluoromethyl and the like.

“Haloaryl,” which is an example of a substituted aryl, means an aryl having at least one hydrogen atom replaced with halogen, such as 4-fluorophenyl and the like.

“Alkoxy” means an alkyl moiety attached through an oxygen bridge (i.e., —O-alkyl) such as methoxy, ethoxy, and the like.

“Haloalkoxy,” which is an example, of a substituted alkoxy, means an alkoxy moiety having at least one hydrogen atom replaced with halogen, such as chloromethoxy and the like.

“Alkoxydiyl” means an alkyl moiety attached through two separate oxygen bridges (i.e., —O-alkyl-O—) such as —O—CH₂—O—, —O—CH₂CH₂—O—, —O—CH₂CH₂CH₂—O—, —O—CH(CH₃)CH₂CH₂—O—, —O—CH₂C(CH₃)₂CH₂—O—, and the like.

“Alkanediyl” means a divalent alkyl from which two hydrogen atoms are taken from the same carbon atom or from different carbon atoms, such as —CH₂—, —CH₂CH₂—, —CH₂CH₂CH₂—, —CH(CH₃)CH₂CH₂—, —CH₂C(CH₃)₂CH₂—, and the like.

“Thioalkyl” means an alkyl moiety attached through a sulfur bridge (i.e., —S-alkyl) such as methylthio, ethylthio, and the like.

“Alkylamino” and “dialkylamino” mean one or two alkyl moieties attached through a nitrogen bridge (i.e., —N-alkyl) such as methylamino, ethylamino, dimethylamino, diethylamino, and the like.

“Carbamate” is RₐOC(═O)NRₐR_{b}.

“Cyclic carbamate” means any carbamate moiety that is part of a ring.

“Amidyl” is —NRₐR_{b}.

“Hydroxyl” or “hydroxy” refers to the —OH radical.

“Sulfhydryl” or “thio” is —SH.

“Amino” refers to the —NH₂ radical.

“Nitro” refers to the —NO₂ radical.

“Imino” refers to the ═NH radical.

“Thioxo” refers to the ═S radical.

“Cyano” refers to the —C≡N radical.

“Sulfonamide refers to the radical —S(═O)₂NH₂.

“Isocyanate” refers to the —N═C═O radical.

“Isothiocyanate” refers to the —N═C═S radical.

“Azido” refers to the —N═N⁺═N⁻ radical.

“Carboxy” refers to the —CO₂H radical (also depicted as —C(═O)OH or COOH).

“Hydrazide” refers to the —C(═O)NRₐ—NRₐR_{b} radical.

“Oxo” refers to the ═O radical.

The compounds described herein may generally be used as the free acid or free base. Alternatively, the compounds may be used in the form of acid or base addition salts. Acid addition salts of the free base amino compounds may be prepared according to methods well known in the art, and may be formed from organic and inorganic acids. Suitable organic acids include (but are not limited to) maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include (but are not limited to) hydrochloric, hydrobromic, sulfuric, phosphoric, and nitric acids. Base addition salts of the free acid compounds of the compounds described herein may also be prepared by methods well known in the art, and may be formed from organic and inorganic bases. Suitable inorganic bases included (but are not limited to) the hydroxide or other salt of sodium, potassium, lithium, ammonium, calcium, magnesium, iron, zinc, copper, manganese, aluminum, and the like, and organic bases such as substituted ammonium salts. Thus, the term “pharmaceutically acceptable salt” of compounds of Structures I and II and substructures thereof, as well as any and all substructures and specific compounds described herein is intended to encompass any and all pharmaceutically suitable salt forms.

Compounds of Structures I and II and substructures thereof may sometimes be depicted as an anionic species. For instance, the compounds may be depicted as the sulfonic acid (SO₃⁻) anion. One of ordinary skill in the art will recognize that the compounds exist with an equimolar ratio of cation. For instance, the compounds described herein can exist in the fully protonated form, or in the form of a salt such as sodium, potassium, ammonium or in combination with any inorganic base as described above. When more than one anionic species is depicted, each anionic species may independently exist as either the protonated species or as the salt species. In some specific embodiments, the compounds described herein exist as the sodium salt.

Also contemplated are prodrugs of compounds of structure I and substructure thereof and structure II and substructures thereof described herein. Prodrugs are any covalently bonded carriers that release the compound of Structure I or II, or substructures thereof, as described herein, in vivo when such prodrug is administered to a subject. Prodrugs are generally prepared by modifying functional groups in a way such that the modification is cleaved, either by routine manipulation or by an in vivo process, yielding the parent compound. Prodrugs include, for example, thiazolidinone derivative compounds of structure I (and substructures thereof) and glycine hydrazide compounds of structure II (and substructures thereof) described herein when, for example, hydroxy or amine groups are bonded to any group that, when administered to a subject, is cleaved to form the hydroxy or amine groups. Thus, representative examples of prodrugs include (but are not limited to) acetate, formate and benzoate derivatives of alcohol and amine functional groups of the compounds of Structures I and II, and substructures thereof, as described herein. Further, in the case of a carboxylic acid (—COOH), esters may be employed, such as methyl esters, ethyl esters, and the like. Prodrug chemistry is conventional to and routinely practiced by a person having ordinary skill in the art.

Prodrugs are typically rapidly transformed in vivo to yield the parent thiazolidinone derivative compounds of structure I (and substructures thereof) or the parent glycine hydrazide compounds of structure II (and substructures thereof), for example, by hydrolysis in blood. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

With regard to stereoisomers, the compounds of structure I and substructures thereof and structure II and substructures thereof, may have one or more chiral centers and may occur in any isomeric form, including racemates, racemic mixtures, and as individual enantiomers or diastereomers. In addition, the compounds of structure I and substructures thereof and structure II and substructures thereof that contain olefinic double bonds or other centers of geometric asymmetry, unless specifically indicated otherwise, include both E and Z geometric isomers (e.g., cis or trans). Accordingly, in such structures with an olefinic double bond or other center of geometric asymmetry, a bond shown as a wavy bond or a bond shown as a straight bond each indicate that both E and Z geometric isomers are included. Likewise, all possible isomers, as well as their racemic and optically pure forms, and all tautomeric forms are also intended to be included. A tautomer refers to a proton shift from one atom of a molecule to another atom of the same molecule. All such isomeric forms of the compounds are included and contemplated, as well as mixtures thereof. Furthermore, some of the crystalline forms of any compound described herein may exist as polymorphs, which are also included and contemplated by the present disclosure. In addition, some of the compounds may form solvates with water or other organic solvents. Such solvates are similarly included within the scope of compounds and compositions described herein.

In general, the compounds used in the reactions described herein may be made according to organic synthesis techniques known to those skilled in this art, starting from commercially available chemicals and/or from compounds described in the chemical literature. “Commercially available chemicals” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hanover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

Methods known to one of ordinary skill in the art may be identified through various reference books and databases. Suitable reference books and treatises that detail the synthesis of reactants useful in the preparation of thiazolidinone derivative compounds of structure I (and substructures thereof) and glycine hydrazide compounds of structure II (and substructures thereof) described herein, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions,” 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the thiazolidinone derivative compounds of structure I (and substructures thereof) and glycine hydrazide compounds of structure II (and substructures thereof) described herein is P. H. Stahl & C. G. Wermuth “Handbook of Pharmaceutical Salts”, Verlag Helvetica Chimica Acta, Zurich, 2002.

### Synthesis of Compounds of Structures I and II

Synthesis of thiazolidinone compounds of structure I and substructures I(A), I(A1-A8), I(B), I(B1), I(C), I(C1) and specific structures as described herein may be performed by general methods described herein (see Example 1) and in the art (see, e.g., Ma et al., J. Clin. Invest. 110, 1651-1658 (2002); Sonawane et al., J. Pharm. Sci. 94: 134-143 (2004); U.S. Pat. No. 7,235,573; see also, e.g., U.S. Pat. No. 5,326,770 and U.S. Pat. No. 6,380,186). Synthesis of glycine hydrazide derivative compounds of structure II and substructures II(A) and II(B) and specific structures described herein may be performed as described herein and in the art (see, e.g., U.S. Pat. No. 7,414,037, U.S. Patent Application Publication No. 2005/0239740; Muanprasat et al., J. Gen. Physiol. 124:125-137 (2004)).

Those skilled in the art will also appreciate that in the processes described herein and in the art, functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters. Protecting groups may be added or removed in accordance with standard techniques, which are well-known to those skilled in the art and as described herein. The use of protecting groups is described in detail in Theodora W. Greene, Peter G. M. Wuts, Protective Groups in Organic Synthesis (1999), 3rd Ed., Wiley-Interscience. The protecting group may also be a polymer resin such as a Wang resin or a 2-chlorotrityl chloride resin.

The following Reaction Scheme illustrates methods to make compounds described herein. A person of ordinary skill in the art would be able to make the compounds these compounds by similar methods or by methods known to one skilled in the art. In general, starting components may be obtained from sources such as Sigma-Aldrich (St. Louis, Mo.), or synthesized according to sources known to those of ordinary skill in the art (see, e.g., Smith and March, March's Advanced Organic Chemistry: Reactions, Mechanisms, and Structure, 5th edition (Wiley Interscience, New York) and other publications described herein). Moreover, the various substituted groups (e.g., R₁, R₂, R₃, and X₁, etc.) of the compounds of the invention may be attached to the starting components, intermediate components, and/or final products according to methods known to those of ordinary skill in the art.

### Synthesis of Thiazolidinone Compounds of Structure I

An exemplary reaction scheme for preparation of thiazolidinone derivative compounds is provided in Scheme 1. Route 1 of Scheme 1 has been described for synthesis of CFTRᵢₙₕ-172 (Compound 5) and CFTRᵢₙₕ-172 derivatives (see, e.g., Ma et al., J. Clin. Invest. 110, 1651-1658 (2002); U.S. Pat. No. 7,235,573; U.S. Patent Application Publication No. US2008/0064666); Sonawane et al., Biorg. Med. Chem. 16:8187-95 (2008)).

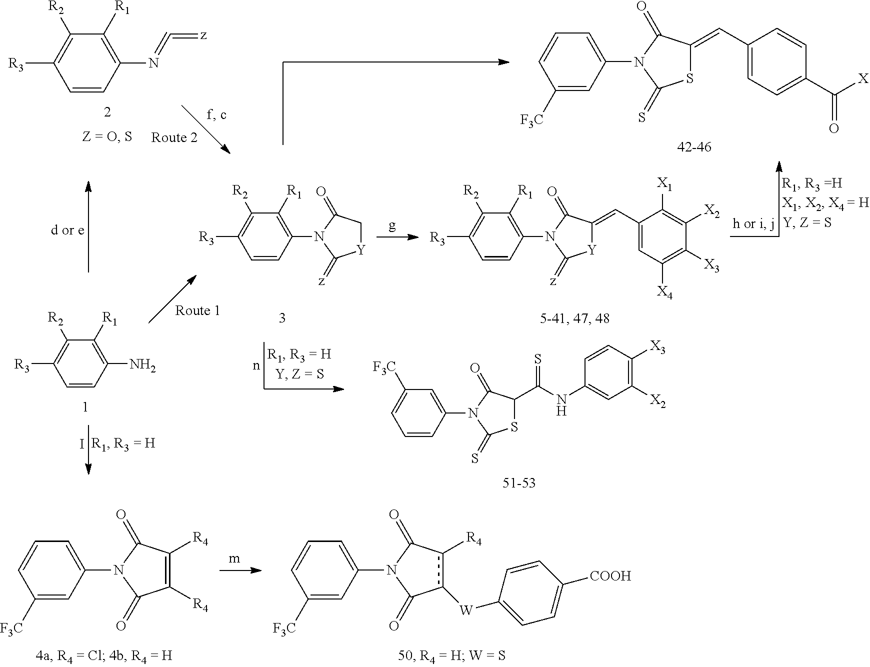

Reagents and conditions that are used are described in greater detail herein (See Example 1). For synthesis of thiazolidinone intermediates by route 2 as shown in Scheme 1, certain isocyanates and isothiocyanates are commercially available.

Equimolar carbon disulfide is added to an ice-cold solution of 3-trifluoromethylaniline and triethylamine in ethyl acetate over a period of time (see Scheme 1). After stirring for a period of time between about 1 hour to 24 hours, a yellow dithiocarbamate is isolated by filtration and reacted with equimolar amount of aqueous bromoacetic acid solution. After a period of time (about 1-4 hours), the solution is acidified, refluxed, and the resultant precipitate is crystallized from ethanol to yield the thiazolidinone intermediate. Intermediates may be confirmed by mass and ¹H NMR.

Scheme 1 also shows an alternate isothiocyanate route (Route 2) that may be used for synthesis of the 2-thiaoxo-4-thiazolidinone ring intermediates 3. Isothiocyanates 2 may be prepared by single step reaction of corresponding amino compounds 1 with thiophosgene and reacted with thioglycolic acid in the presence of triethylamine to yield dithiocarbamate intermediates. The intermediates upon in situ acidification and reflux generate 2-thioxox-4-thiazolidinones 3. Route 2 is single-pot. This route may be used for the synthesis of ortho-substituted analogues such as the compound referred to herein as α-Me-172, Compound 18, with high yields. A solution of isothiocyanate 2 in an appropriate solvent, such as THF, is added dropwise to a stirred aqueous solution of thioglycolic acid and triethylamine. After a period of time (e.g., 30 minutes) under conditions sufficient to cool the reaction mixture, the reaction mixture is further stirred at room temperature for a period of time, such as from about 1-6 hours. The reaction mixture is acidified, refluxed, and resultant precipitate crystallized from ethanol to yield thiazolidinone intermediate 3.

Isothiocyanates and isocyanates 2, if not available commercially, may be prepared by reaction of the respective amino compounds 1 with phosgene or thiophosgene, following known procedures.

For compounds that include a tetrazolo group, synthesis of the tetrazolo group may be performed as described in the art (see, e.g., Rostovtsev et al., Angew. Chem. Int. Ed. 41:2596-99 (2002)).

### Methods for Characterizing and Using the Thiazolidinone Derivative Compounds and Glycine Hydrazide Derivative Compounds

The thiazolidinone derivative compounds of structure I and substructures I(A), I(A1-A8), I(B), I(B1), I(C), I(C1) and specific structures as described herein and glycine hydrazide derivative compounds of structure II and substructures II(A) and II(B) and specific structures described herein are capable of blocking or impeding the CFTR pore or channel and inhibiting ion transport (e.g., inhibiting chloride ion (Cl⁻) transport (also referred to as inhibiting chloride ion conductance)) by CFTR located in the outer cell membrane of a cell.

Also provided herein are methods of inhibiting ion transport by CFTR, which comprises contacting a cell that has CFTR in the outer membrane with any one of the compounds described herein, thus permitting CFTR and the compound or compounds to interact. As described herein interaction of the thiazolidone compounds and glycine hydrazide compounds described herein results in binding to CFTR thereby inhibiting chloride ion transport.

In certain embodiments, these methods may be performed in vitro, such as with using a biological sample as described herein that comprises, for example, cells obtained from a tissue, body fluid, or culture adapted cell line or other biological source as described in detail herein below. The step of contacting refer to combining, mixing, or in some manner familiar to persons skilled in the art that permits the compound and the cell to interact such that any effect of the compound on CFTR activity can be measured according to methods described herein and routinely practiced in the art. Methods described herein for inhibiting ion transport by CFTR are understood to be performed under conditions and for a time sufficient that permit the CFTR and the compound to interact. Thiazolidinone derivative compounds of structure I (and substructures thereof) and glycine hydrazide compounds of structure II (and substructures thereof) may be identified and/or characterized by such a method of inhibiting ion transport by CFTR, performed with isolated cells in vitro. Conditions for a particular assay include temperature, buffers (including salts, cations, media), and other components that maintain the integrity of the cell and the compound, which a person skilled in the art will be familiar and/or which can be readily determined. A person skilled in the art also readily appreciates that appropriate controls can be designed and included when performing the in vitro methods and in vivo methods described herein.

Without wishing to be bound by any particular theory, in secretory epithelia, fluid secretion occurs by primary chloride exit across the cell apical membrane, which secondarily drives transepithelial sodium and water secretion (see, e.g., Barrett et al., Annu. Rev. Physiol. 62:535-72 (2000)). In renal cells, lumenal fluid accumulation causes progressive cyst expansion directly by net water influx into the cyst lumen, and indirectly by stretching cyst wall epithelial cells to promote their division and thinning (Ye et al., N. Engl. J. Med. 329:310-13 (1993); Sullivan et al., Physiol. Rev. 78:1165-91 (1998); Tanner et al., J. Am. Soc. Nephrol. 6:1230-41 (1995)). CFTR inhibition interferes with fluid secretion at the apical chloride exit step.

Methods for characterizing a compound, such as determining an effective concentration to achieve a therapeutic benefit, may be performed using techniques and procedures described herein and routinely practiced by a person skilled in the art. Exemplary methods include, but are not limited to, fluorescence cell-based assays of CFTR inhibition (see, e.g., Galietta et al., J. Physiol. 281:C1734-C1742 (2001)), short circuit apical chloride ion current measurements and patch-clamp analysis (see, e.g., Muanprasat et al., J. Gen. Physiol. 124:125-37 (2004); Ma et al., J. Clin. Invest. 110:1651-58 (2002); see also, e.g., Carmeliet, Verh. K. Acad. Geneeskd. Belg. 55:5-26 (1993); Hamill et al., Pflugers Arch. 391:85-100 (1981)). The thiazolidinone and glycine hydrazide compounds may also be analyzed in animal models, for example, a closed intestinal loop model of cholera, suckling mouse model of cholera, and in vivo imaging of gastrointestinal transit (see, e.g., Takeda et al., Infect. Immun. 19:752-54 (1978); see also, e.g., Spira et al., Infect. Immun. 32:739-747 (1981)). See also Yang et al., J. Am. Soc. Nephrol. 19:1300-1310 (2008).

Methods that may be used to characterize a thiazolidinone derivative compound or a glycine derivative compound, including those described herein, and to determine effectiveness of the compound for reducing, inhibiting, or preventing cyst enlargement and/or preventing or inhibiting cyst formation, and which compound is therefore useful for treating a subject who has or who is at risk of developing PKD, include methods described in the art and herein. For example, a cell culture model for determining whether a compound inhibits cyst formation or enlargement includes an MDCK cell (Madin-Darby Canine Kidney Epithelial Cell) model of PKD (Li et al., Kidney Int 66:1926-1938 (2004); see also, e.g., Neufeld et al., Kidney Int. 41:1222-36 (1992); Mangoo-Karim et al., Proc. Natl. Acad. Sci. USA 86:6007-6011 (1989); Mangoo-Karim et al., FASEB J. 3:2629-32 (1989); Grantham et al., Trans. Assoc. Am. Physic. 102:158-62 (1989); Mohamed et al., Biochem J 322: 259-265 (1997)). See also, e.g., Murcia et al., Kidney Int. 55:1187-97 (1999); Igarishi et al., J. Am. Soc. Nephrol. 13:2384-88 (2002)). Accordingly, provided herein are methods for identifying or characterizing thiazolidinone derivative compounds of structure I (and substructures thereof) and glycine hydrazide compounds of structure II (and substructures thereof) by determining the capability of the compound to inhibit cyst enlargement or prevent or inhibit cyst formation in an in vitro cell culture model.

The MDCK cell line may also be used in methods and techniques for determining that a compound lacks cytotoxicity, for example, by evaluating cell viability (e.g., by any one of numerous cell staining methods and microscopy methods routinely practiced in the art), cell proliferation (e.g., by determining the level of incorporation of nucleotide analogs and other methods for measuring division of cells), and/or apoptosis by using any one of a number of techniques and methods known in the art and described herein.

Other methods for determining or quantifying the capability of a compound described herein to inhibit cyst enlargement or expansion and/or to inhibit or prevent cyst formation include an embryonic kidney organ culture model, which is practiced in the art and described herein (see, e.g., Magenheimer et al., J. Am. Soc. Nephrol. 17: 3424-37 (2006); Steenhard et al., J. Am. Soc. Nephrol. 16:1623-1631 (2005)). In such an embryonic kidney culture model, organotypic growth and differentiation of renal tissue can be monitored in defined media in the absence of any effect or influence by circulating hormones and glomerular filtration (Magenheimer et al., supra; Gupta et al., Kidney Int. 63:365-376 (2003)). In metanephric organ culture, the early mouse kidney tubule has an intrinsic capacity to secrete fluid by a CFTR-dependent mechanism in response to cAMP (Magenheimer et al., supra).

Persons skilled in the art may also use animal models to characterize a thiazolidinone derivative compound or a glycine derivative compound, including those described herein, and to determine effectiveness of the compound for reducing, inhibiting, or preventing cyst enlargement and/or preventing or inhibiting cyst formation, and to determine the usefulness of such compounds for treating a subject who has or who is at risk of developing PKD. By way of example, Pkd1^{flox} mice and Ksp-Cre transgenic mice in a C57BL/6 background may be generated as described and practiced in the art (see, e.g., Shibazaki et al., J. Am. Soc. Nephrol. 13:10-11 (2004) (abstract); Shao et al., J. Am. Soc. Nephrol. 13:1837-46 (2002)). Ksp-Cre mice express Cre recombinase under the control of the Ksp-cadherin promoter (see, e.g., Shao et al., supra). Pkd1^{flox/-}; Ksp-Cre mice may be generated by cross-breeding Pkd1^{flox/flox} mice with Pkd1^{+/-}:Ksp-Cre mice. The effect of a test compound may be determined by quantifying cyst size and growth in metanephroi and kidney sections, histological analyses of tissues and cells, and delay or prevention of renal failure and death (see, e.g., Shibazaki et al., supra).

As described herein, the thiazolidinone derivative compounds of structure I and substructures I(A), I(A1-A8), I(B), I(B1), I(C), I(C1) and specific structures as described herein and glycine hydrazide derivative compounds of structure II and substructures II(A) and II(B) and specific structures described herein are capable of inhibiting CFTR activity (i.e., inhibiting, reducing, decreasing, blocking transport of chloride ion in the CFTR channel or pore in a statistically significant or biologically significant manner) in a cell and may be used for treating diseases, disorders, and conditions that result from or are related to aberrantly increased CFTR activity. Accordingly, methods of inhibiting ion transport by CFTR are provided herein that comprise contacting a cell (e.g., a gastrointestinal cell) that comprises CFTR in the outer membrane of the cell (i.e., a cell that expresses CFTR and has channels or pores formed by CFTR in the cell membrane) with any one or more of the thiazolidinone derivative compounds of structure I (and substructures thereof) and glycine hydrazide compounds of structure II (and substructures thereof) described herein, under conditions and for a time sufficient for CFTR and the compound to interact.

In certain embodiments, the cell is contacted in an in vitro assay, and the cell may be obtained from a subject or from a biological sample. A biological sample may be a blood sample (from which serum or plasma may be prepared and cells isolated), biopsy specimen, body fluids (e.g., lung lavage, ascites, mucosal washings, synovial fluid), bone marrow, lymph nodes, tissue explant, organ culture, or any other tissue or cell preparation from a subject or a biological source. A sample may further refer to a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. The subject or biological source may be a human or non-human animal, a primary cell culture (e.g., immune cells, virus infected cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein the thiazolidinone derivative compounds of structure I (and substructures thereof) and glycine hydrazide compounds of structure II (and substructures thereof) are CFTR inhibitors, and are useful in the treatment of a CFTR-mediated or associated condition, i.e., any condition, disorder or disease, that results from activity of CFTR, such as CFTR activity in ion transport. Such conditions, disorders, and diseases, are amenable to treatment by inhibition of CFTR activity, e.g., inhibition of CFTR ion transport.

In one embodiment, the thiazolidinone derivative compounds of structure I (and substructures thereof) and glycine hydrazide compounds of structure II (and substructures thereof) are used in the treatment of conditions associated with aberrantly increased intestinal secretion, particularly acute aberrantly increased intestinal secretion, including secretory diarrhea. Diarrhea amenable to treatment using thiazolidinone derivative compounds of structure I (and substructures thereof) and glycine hydrazide compounds of structure II (and substructures thereof) can result from exposure to a variety of pathogens or agents including, without limitation, cholera toxin (Vibrio cholera), E. coli (particularly enterotoxigenic (ETEC)), Shigella, Salmonella, Campylobacter, Clostridium difficile, parasites (e.g., Giardia, Entamoeba histolytica, Cryptosporidiosis, Cyclospora), or diarrheal viruses (e.g., rotavirus). Secretory diarrhea resulting from an increased intestinal secretion mediated by CFTR may also be a disorder or sequelae associated with food poisoning, or exposure to a toxin including an enterotoxin such as cholera toxin, a E. coli toxin, a Salmonella toxin, a Campylobacter toxin, or a Shigella toxin.

Other secretory diarrheas that may be treated by administering any one or more of the thiazolidinone derivative compounds of structure I (and substructures thereof) and glycine hydrazide compounds of structure II (and substructures thereof) described herein include diarrhea associated with or that is a sequelae of AIDS, diarrhea that is a condition related to the effects of anti-AIDS medications such as protease inhibitors, diarrhea that is a condition or is related to administration of chemotherapeutic compounds, inflammatory gastrointestinal disorders, such as ulcerative colitis, inflammatory bowel disease (IBD), Crohn's disease, diverticulosis, and the like. Intestinal inflammation modulates the expression of three major mediators of intestinal salt transport and may contribute to diarrhea in ulcerative colitis both by increasing transepithelial Cl⁻ secretion and by inhibiting the epithelial NaCl absorption (see, e.g., Lohi et al., Am. J. Physiol. Gastrointest. Liver Physiol. 283:G567-75 (2002)).

Thus, one or more of the thiazolidinone derivative compounds of structure I and substructures I(A), I(A1-A8), I(B), I(B1), I(C), I(C1) and specific structures as described herein and glycine hydrazide derivative compounds of structure II and substructures II(A) and II(B) and specific structures described herein may be administered in an amount effective to inhibit CFTR ion transport and, thus, decrease intestinal fluid secretion. In such embodiments, at least one or more of the compounds are generally administered to a mucosal surface of the gastrointestinal tract (e.g., by an enteral route, e.g., oral, intraintestinal, rectal, and the like) or to a mucosal surface of the oral or nasal cavities, or (e.g., intranasal, buccal, sublingual, and the like).

Methods are provided herein for treating a disease or disorder associated with aberrantly increased ion transport by cystic fibrosis transmembrane conductance regulator (CFTR) and that is treatable by inhibiting ion transport by CFTR, wherein the methods comprise administering to a subject any one (or more) of the thiazolidinone derivative compounds of structure I (and substructures thereof) and glycine hydrazide compounds of structure II (and substructures thereof) described herein, wherein ion transport (particularly chloride ion transport) by CFTR is inhibited.

Other embodiments provided herein include use of at least one of the thiazolidinone derivative compounds of structure I and substructures I(A), I(A1-A8), I(B), I(B1), I(C), I(C1) and specific structures as described herein and glycine hydrazide derivative compounds of structure II and substructures II(A) and II(B) and specific structures described herein for treating any one of the diseases or disorders described herein that is treatable by inhibiting ion transport (e.g., chloride ion transport) by CFTR. In one embodiment, a use is provided for the preparation of a medicament for treating any one of the diseases or disorders described herein that is treatable by inhibiting ion transport (e.g., chloride ion transport) by CFTR.

A subject in need of the treatments described herein includes humans and non-human animals. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

### Pharmaceutical Compositions

Also provided herein are pharmaceutical compositions comprising any one or more of the thiazolidinone derivative compounds of structure I (and substructures thereof) and/or glycine hydrazide compounds of structure II (and substructures thereof). The compounds described herein may be formulated in a pharmaceutical composition for use in treatment, which includes preventive treatment, of a disease or disorder manifested by increased intestinal fluid secretion, such as secretory diarrhea. In other embodiments, the compounds described herein may be formulated in a pharmaceutical composition for use in treatment, which includes preventive treatment, of polycystic kidney disease (PKD), which includes autosomal dominant PKD (ADPKD) and autosomal recessive PKD (ARPKD).

In pharmaceutical dosage forms, any one or more of the thiazolidinone derivative compounds of structure I and substructures I(A), I(A1-A8), I(B), I(B1), I(C), I(C1) and specific structures as described herein and glycine hydrazide derivative compounds of structure II and substructures II(A) and II(B) and specific structures described herein may be administered in the form of a pharmaceutically acceptable derivative, such as a salt, or they may also be used alone or in appropriate association, as well as in combination, with other pharmaceutically active compounds. The methods and excipients described herein are merely exemplary and are in no way limiting.

In one embodiment of particular interest, the thiazolidinone derivative compounds of structure I (and substructures thereof) and/or glycine hydrazide compounds of structure II (and substructures thereof) are delivered to the gastrointestinal tract of the subject to provide for decreased fluid secretion. Suitable formulations for this embodiment include any formulation that provides for delivery of the compound to the gastrointestinal surface, particularly an intestinal tract surface.

Optimal doses may generally be determined using experimental models and/or clinical trials. The optimal dose may depend upon the body mass, weight, or blood volume of the subject. In general, the amount of a thiazolidinone derivative compound of structure I (and substructures thereof) and/or glycine hydrazide compound of structure II (and substructures thereof) described herein, that is present in a dose, ranges from about 0.01 μg to about 1000 μg per kg weight of the host. The use of the minimum dose that is sufficient to provide effective therapy is usually preferred. Subjects may generally be monitored for therapeutic effectiveness using assays suitable for the condition being treated or prevented, which assays will be familiar to those having ordinary skill in the art and are described herein. The level of a compound that is administered to a subject may be monitored by determining the level of the compound in the urine. Any method practiced in the art to detect the compound may be used to measure the level of compound during the course of a therapeutic regimen.

The dose of the composition for treating a disease or disorder associated with aberrant CFTR function, including but not limited to intestinal fluid secretion, secretory diarrhea, such as a toxin-induced diarrhea, or secretory diarrhea associated with or a sequelae of an enteropathogenic infection, Traveler's diarrhea, ulcerative colitis, irritable bowel syndrome (IBS), AIDS, chemotherapy and other diseases or conditions described herein may be determined according to parameters understood by a person skilled in the medical art. Accordingly, the appropriate dose may depend upon the subject's condition, that is, stage of the disease, general health status, as well as age, gender, and weight, and other factors considered by a person skilled in the medical art. Similarly, the dose of a composition comprising at least one of the thiazolidinone derivative compounds and/or glycine hydrazide derivative compounds described herein for treating PKD may depend upon the subject's condition, that is, stage of the disease, renal function, severity of symptoms caused by enlarged cysts, general health status, as well as age, gender, and weight, and other factors apparent to a person skilled in the medical art.

Pharmaceutical compositions may be administered in a manner appropriate to the disease or disorder to be treated as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose (or effective dose) and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). When a subject is treated for aberrantly increased intestinal fluid secretion, clinical assessment of the level of dehydration and/or electrolyte imbalance may be performed to determine the level of effectiveness of a compound and whether dose or other administration parameters (such as frequency of administration or route of administration) should be adjusted.

Polycystic kidney disease (PKD) (or PCKD) and polycystic renal disease are used interchangeably, and refer to a group of disorders characterized by a large number of cysts distributed throughout enlarged kidneys. The resultant cyst development leads to impairment of kidney function and can eventually cause kidney failure. PDK includes autosomal dominant polycystic kidney disease (ADPKD) and recessive autosomal recessive polycystic kidney disease (ARPKD), in all stages of development, regardless of the underlying etiology or cause. Effectiveness of a treatment for PKD may be monitored by one or more of several methods practiced in the medical art including, for example, by monitoring renal function by standard measurements, and by radiologic investigations that are performed with ultrasounds, computerized tomography (CT), or magnetic resonance imaging, which are useful for evaluating renal cyst morphology and volume and estimating the amount of residual renal parenchyma.

To evaluate and to monitor the effectiveness of any one of the compounds described herein to treat PKD or a related disease or condition, one or more of several clinical assay methods may be performed that are familiar to a person skilled in the clinical art. For example, a clinical method called a urea clearance test may be performed. A blood sample is obtained from a subject to whom the compound is being administered so that the amount of urea in the bloodstream can be determined. In addition, a first urine sample is collected from the subject and at least one hour later, a second urine sample is collected. The amount of urea quantified in the urine indicates the amount of urea that is filtered, or cleared by the kidneys into the urine. Another clinical assay method measures urine osmolality (i.e., the amount of dissolved solute particles in the urine). Inability of the kidneys to concentrate the urine in response to restricted fluid intake, or to dilute the urine in response to increased fluid intake during osmolality testing may indicate decreased kidney function.

Urea is a by-product of protein metabolism and is formed in the liver. Urea is then filtered from the blood and excreted in the urine by the kidneys. The BUN (blood urea nitrogen) test measures the amount of nitrogen contained in the urea. High BUN levels may indicate kidney dysfunction, but because blood urea nitrogen is also affected by protein intake and liver function, the test is usually performed in conjunction with determination of blood creatinine, which is considered a more specific indicator of kidney function. Low clearance values for creatinine and urea indicate diminished ability of the kidneys to filter these waste products from the blood and excrete them in the urine. As clearance levels decrease, blood levels of creatinine and urea nitrogen increase. An abnormally elevated blood creatinine, a more specific and sensitive indicator of kidney disease than the BUN, is diagnostic of impaired kidney function.

The terms, “treat” and “treatment” refer to both therapeutic treatment and prophylactic or preventative measures, wherein the object is to prevent or slow or retard (lessen) an undesired physiological change or disorder, or to prevent or slow or retard (lessen) the expansion or severity of such disorder. As discussed herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival when compared to expected survival if a subject were not receiving treatment. Subjects in need of treatment include those already with the condition or disorder as well as subjects prone to have or at risk of developing the condition or disorder, and those in which the condition or disorder is to be prevented.

A pharmaceutical composition may be a sterile aqueous or non-aqueous solution, suspension or emulsion, which additionally comprises a physiologically acceptable excipient (pharmaceutically acceptable or suitable excipient or carrier) (i.e., a non-toxic material that does not interfere with the activity of the active ingredient). Such compositions may be in the form of a solid, liquid, or gas (aerosol). Alternatively, compositions described herein may be formulated as a lyophilizate, or compounds may be encapsulated within liposomes using technology known in the art. Pharmaceutical compositions may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable excipient or carrier known to those of ordinary skill in the art for use in pharmaceutical compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient is selected based on the mode of administration. Pharmaceutical compositions may be formulated for any appropriate manner of administration, including, for example, topical, oral, nasal, intrathecal, rectal, vaginal, intraocular, subconjunctival, sublingual or parenteral administration, including subcutaneous, intravenous, intramuscular, intrasternal, intracavernous, intrameatal or intraurethral injection or infusion. For parenteral administration, the carrier preferably comprises water, saline, alcohol, a fat, a wax or a buffer. For oral administration, any of the above excipients or a solid excipient or carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

A composition comprising any one of the thiazolidinone derivative compounds of structure I (and substructures thereof) and/or glycine hydrazide compounds of structure II (and substructures thereof) described herein may be formulated for sustained or slow release. Such compositions may generally be prepared using well known technology and administered by, for example, oral, rectal or subcutaneous implantation, or by implantation at the desired target site. Sustained-release formulations may contain a compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active compound contained within a sustained release formulation depends upon the site of implantation, the rate and expected duration of release, and the nature of the condition to be treated or prevented.

For oral formulations, the thiazolidinone derivative compounds of structure I (and substructures thereof) and/or glycine hydrazide compounds of structure II (and substructures thereof) described herein can be used alone or in combination with appropriate additives to make tablets, powders, granules or capsules, for example, with conventional additives, such as lactose, mannitol, corn starch or potato starch; with binders, such as starch, gelatin, natural sugars such as glucose or beta-lactose, corn sweeteners, natural and synthetic gums such as acacia, tragacanth, or sodium alginate, carboxymethylcellulose, polyethylene glycol, waxes, crystalline cellulose, cellulose derivatives, and acacia; with disintegrators, such as corn starch, potato starch or sodium carboxymethylcellulose, methyl cellulose, agar, bentonite, or xanthan gum; with lubricants, such as talc, sodium oleate, magnesium stearate sodium stearate, sodium benzoate, sodium acetate, or sodium chloride; and if desired, with diluents, buffering agents, moistening agents, preservatives, coloring agents, and flavoring agents. The compounds may be formulated with a buffering agent to provide for protection of the compound from low pH of the gastric environment and/or an enteric coating. The thiazolidinone derivative compounds of structure I (and substructures thereof) and/or glycine hydrazide compounds of structure II (and substructures thereof) may be formulated for oral delivery with a flavoring agent, e.g., in a liquid, solid or semi-solid formulation and/or with an enteric coating.

Oral formulations may be provided as gelatin capsules, which may contain the active compound along with powdered carriers, such as lactose, starch, cellulose derivatives, magnesium stearate, stearic acid, and the like. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract. Liquid dosage forms for oral administration may contain coloring and/or flavoring agents to increase acceptance of the compound by the subject.

The thiazolidinone derivative compounds of structure I (and substructures thereof) and glycine hydrazide compounds of structure II (and substructures thereof) described herein can be made into suppositories by mixing with a variety of bases such as emulsifying bases or water-soluble bases. The compounds described herein can be administered rectally via a suppository. The suppository can include vehicles such as cocoa butter, carbowaxes and polyethylene glycols, which melt at body temperature, yet are solidified at room temperature.

The thiazolidinone derivative compounds of structure I (and substructures thereof) and glycine hydrazide compounds of structure II (and substructures thereof) described herein may be used in aerosol formulation to be administered via inhalation. The compounds may be formulated into pressurized acceptable propellants such as dichlorodifluoromethane, propane, nitrogen and the like.

Any one or more of the thiazolidinone derivative compounds of structure I (and substructures thereof) and/or glycine hydrazide compounds of structure II (and substructures thereof) described herein may be administered topically (e.g., by transdermal administration). Topical formulations may be in the form of a transdermal patch, ointment, paste, lotion, cream, gel, and the like. Topical formulations may include one or more of a penetrating agent, thickener, diluent, emulsifier, dispersing aid, or binder. When the thiazolidinone derivative compound of structure I (and substructures thereof) and/or glycine hydrazide compound of structure II (and substructures thereof) compound is formulated for transdermal delivery, the compound may be formulated with or for use with a penetration enhancer. Penetration enhancers, which include chemical penetration enhancers and physical penetration enhancers, facilitate delivery of the compound through the skin, and may also be referred to as “permeation enhancers” interchangeably. Physical penetration enhancers include, for example, electrophoretic techniques such as iontophoresis, use of ultrasound (or “phonophoresis”), and the like. Chemical penetration enhancers are agents administered either prior to, with, or immediately following compound administration, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneruas et al., J. Pharm. Pharmacol. 2002; 54(4):499-508; Karande et al., Pharm. Res. 2002; 19(5):655-60; Vaddi et al., Int. J. Pharm. 2002 July; 91(7):1639-51; Ventura et al., J. Drug Target 2001; 9(5):379-93; Shokri et al., Int. J. Pharm. 2001; 228(1-2):99-107; Suzuki et al., Biol. Pharm. Bull. 2001; 24(6):698-700; Alberti et al., J. Control Release 2001; 71(3):319-27; Goldstein et al., Urology 2001; 57(2):301-5; Kiijavainen et al., Eur. J. Pharm. Sci. 2000; 10(2):97-102; and Tenjarla et al., Int. J. Pharm. 1999; 192(2):147-58.

When a thiazolidinone derivative compound of structure I (and substructures thereof) or a glycine hydrazide compound of structure II (and substructures thereof) is formulated with a chemical penetration enhancer, the penetration enhancer is selected for compatibility with the compound, and is present in an amount sufficient to facilitate delivery of the compound through skin of a subject, e.g., for delivery of the compound to the systemic circulation. The thiazolidinone derivative compounds of structure I (and substructures thereof) and/or glycine hydrazide compounds of structure II (and substructures thereof) may be provided in a drug delivery patch, e.g., a transmucosal or transdermal patch, and can be formulated with a penetration enhancer. The patch generally includes a backing layer, which is impermeable to the compound and other formulation components, a matrix in contact with one side of the backing layer, which matrix provides for sustained release, which may be controlled release, of the compound, and an adhesive layer, which is on the same side of the backing layer as the matrix. The matrix can be selected as is suitable for the route of administration, and can be, for example, a polymeric or hydrogel matrix.

For use in the methods described herein, one or more of the thiazolidinone derivative compounds of structure I (and substructures thereof) and/or glycine hydrazide compounds of structure II (and substructures thereof) described herein may be formulated with other pharmaceutically active agents or compounds, including other CFTR-inhibiting agents and compounds or agents and compounds that block intestinal chloride channels. Similarly, one or more of the thiazolidinone derivative compounds of structure I (and substructures thereof) and/or glycine hydrazide compounds of structure II (and substructures thereof) described herein may be formulated with other pharmaceutically active agents or compounds, including other CFTR-inhibiting agents and compounds, or other agents and compounds that are administered to a subject for treating PKD.

Kits with unit doses of the thiazolidinone derivative compounds of structure I (and substructures thereof) and/or glycine hydrazide compounds of structure II (and substructures thereof) described herein, usually in oral or injectable doses, are provided. In such kits, in addition to the containers containing the unit doses, will be an informational package insert describing the use and attendant benefits of the drugs in treating pathological condition of interest.

Also provided herein are methods of manufacturing the pharmaceutical compositions described herein that comprise at least one of the thiazolidinone derivative compounds of structure I and substructures I(A), I(A1-A8), I(B), I(B1), I(C), I(C1) and specific structures as described herein and glycine hydrazide derivative compounds of structure II and substructures II(A) and II(B) and specific structures described herein. In one embodiment, the method of manufacture comprises synthesis of the compound. Synthesis of one of more of the compounds described herein may be performed according to methods described herein and practiced in the art. In another method of manufacture, the method comprises comprise formulating (i.e., combining, mixing) at least one of the compounds disclosed herein with a pharmaceutically suitable excipient. These methods are performed under conditions that permit formulation and/or maintenance of the desired state (i.e., liquid or solid, for example) of each of the compound and excipient. A method of manufacture may comprise one or more of the steps of synthesizing the at least one compound, formulating the compound with at least one pharmaceutically suitable excipient to form a pharmaceutical composition, and dispensing the formulated pharmaceutical composition in an appropriate vessel (i.e., a vessel appropriate for storage and/or distribution of the pharmaceutical composition).

Other embodiments and uses will be apparent to one skilled in the art in light of the present disclosures. The following examples are provided merely as illustrative of various embodiments and shall not be construed to limit the invention in any way.

### EXAMPLES

### Example 1

### Synthesis of Thiazolidinone Derivative Compounds

### Synthesis of Thiazolidinone Compounds of Structure I

An exemplary reaction scheme for preparation of thiazolidinone derivative compounds is provided in Scheme 1. Route 1 of Scheme 1 has been described for synthesis of CFTRᵢₙₕ-172 (Compound 5) and CFTRᵢₙₕ-172 analogues (see, e.g., Ma et al., J. Clin. Invest. 110, 1651-1658 (2002); U.S. Pat. No. 7,235,573); Sonawane et al., Bioorg. Med. Chem. 16:8187-95 (2008)).

¹H nuclear magnetic resonance spectra were obtained in CDCl₃ or dimethyl sulfoxide (DMSO)-d₆ using a 400-MHz Varian Spectrometer referenced to CDCl₃ or DMSO. Mass spectrometry was done on a Waters LC/MS system (Alliance HT 2790+ZQ, HPLC: Waters model 2690, Milford, Mass.). Flash chromatography was done using EM silica gel (230-400 mesh), and thin-layer chromatography was performed on MERK silica gel 60 F254 plates (Darmstadt, Germany).

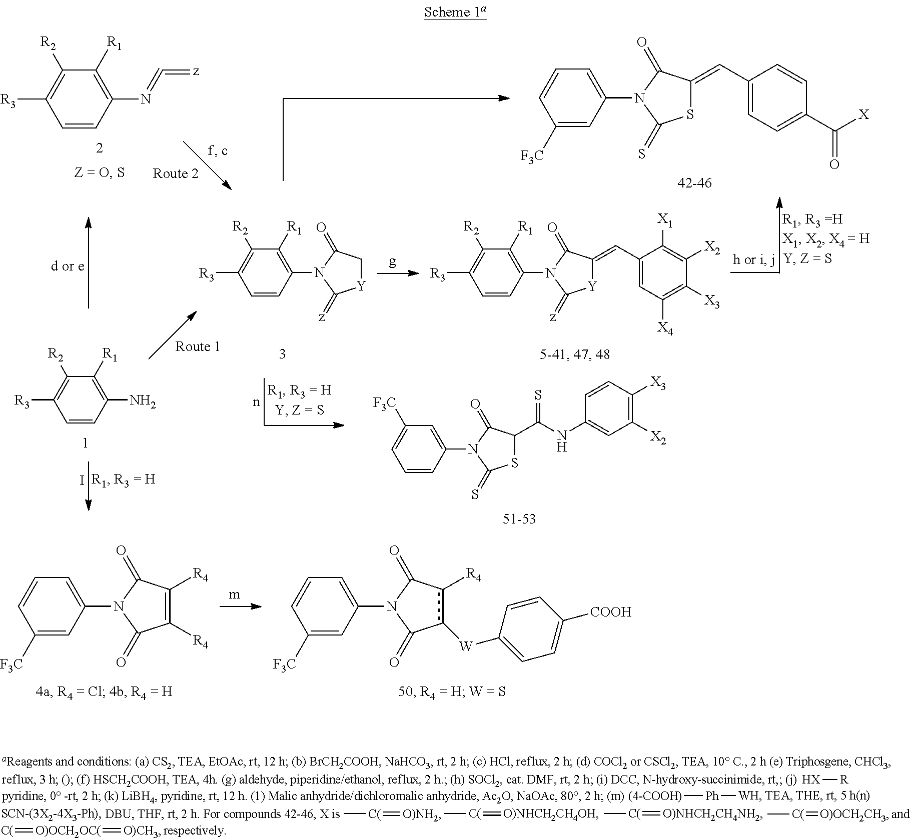

For synthesis of thiazolidinone intermediates by route 2 as shown in Scheme 1, certain isocyanates and isothiocyanates are commercially available.

Briefly, equimolar carbon disulfide was added dropwise to an ice-cold solution of 3-trifluoromethylaniline and triethylamine in ethyl acetate over 30 min (see Scheme 1). After stirring overnight, a yellow dithiocarbamate was isolated by filtration and reacted with equimolar amount of aqueous bromoacetic acid solution (NaHCO₃, pH 8-9). After 2 h, the solution was acidified (HCl), refluxed, and resultant precipitate crystallized from ethanol to yield the thiazolidinone intermediate. Intermediates were confirmed by mass and ¹H NMR. Compounds that had lesser CFTR inhibitor activity or were inactive and their intermediates were characterized by mass spectral analysis (LC/MS). Purity was determined by TLC and HPLC. Compounds with purity >95% purity were used for CFTR inhibition testing.

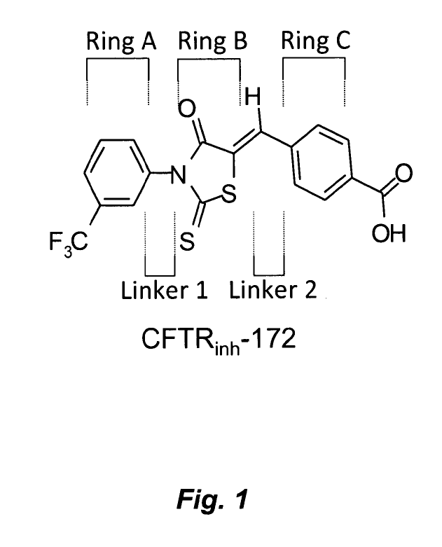

CFTRᵢₙₕ-172 analogs were synthesized with different substituents on Ring A (see FIG. 1), keeping the remainder of the molecule the same. Commercially available substituted anilines 1 were reacted with carbon disulfide, followed by reaction with bromoacetate and acidic cyclization to give thiazolidinone intermediate 3 (Route 1, Scheme 1). These intermediates, upon Knoevenagel condensation with aromatic aldehydes in ethanol under reflux in the presence of piperidine, produced the target CFTRᵢₙₕ-172 analogs 5-44 and 47-49 (see Table 1). TLC and LC/MS showed quantitative formation of products. This reaction generates a double bond that can produce E and Z isomers. Similar analogs have been reported to exist predominantly as Z-isomers (see, e.g., Cutshall et al., Bioorg. Med. Chem. Lett. 15:3374 (2005); Deubner et al., Magn. Reson. Chem. 40:762 (2002); Fresneau et al., J. Med. Chem. 41:4706 (1998): Bulletin et al., Chem. Pharm. Bull. 39:1440 (1991); Ohishi et al., Chem. Pharm. Bull. 38:1911 (1990); Sing et al. Biorg. Med. Chem. Lett. 11:91 (2001)).

Scheme 1 also shows an alternate isothiocyanate route (Route 2) used for synthesis of the 2-thiaoxo-4-thiazolidinone ring intermediates 3. This route was used for the synthesis of ortho-substituted analogues like α-Me-172, Compound 18, with high yields. Route 2 was also used for synthesis of compounds 13, 20, 22, and 27. Isothiocyanates 2 were prepared by single step reaction of corresponding amino compounds 1 with thiophosgene and reacted with thioglycolic acid in the presence of triethylamine to yield dithiocarbamate intermediates. This intermediate upon in situ acidification (HCl) and reflux generated 2-thioxo-4-thiazolidonones 3. Route 2 was single-pot and increased overall yields compared with Route 1. Briefly, a solution of isothiocyante 2 (5 mmol, in THF) was added dropwise to a stirred aqueous solution of thioglycolic acid (0.347 g, 3.7 mmol) and triethylamine (1.38 ml, 10 mmol). After 30 min at 0° C., the reaction mixture was further stirred at room temperature for 3 h. The reaction mixture was acidified (HCl), refluxed, and resultant precipitate crystallized from ethanol to yield thiazolidinone intermediate 3.

For synthesis of compound 50, maleimide intermediates 4 were prepared by reaction of 3-trifluoromethylaniline with dichloromaleic anhydride (R4=CL) or maleic anhydride (R4=H) in refluxing acetic anhydride (Scheme 2). Subsequent reaction with 4-aminobenzoic acid and 4-mercaptobenzoic acid produced compound 50. Compounds 51 and 52 were synthesized by reaction of aryl isothiocyanates with 3 in presence of base DBU at room temperature (Scheme 1).

Isothiocyanates and isocyanates 2, if not available commercially, were prepared by reaction of the respective amino compounds 1 with phosgene or thiophosgene, following known procedures.

### Example 2

### Compound 5: CFTRᵢₙₕ-172

Synthesis of CFTRᵢₙₕ-172 (4-[[4-Oxo-2-thioxo-3-[3-(trifluoromethyl)phenyl]-5-thiazolidinylidene]methyl]benzoic acid) was performed as described in Example 1. See Scheme 1.

A mixture of 2-thioxo-3-(3-trifluoromethyl phenyl)-4-thiazolidinone (prepared as described above for intermediate 3) (55 mg, 0.2 mmol), 4-carboxybenzaldehyde (30 mg, 0.2 mmol), and a drop of piperidine in absolute ethanol (0.5 ml) was refluxed for 2 h. Solvent was evaporated, and the residue was crystallized from ethanol and further purified by normal phase flash chromatography to yield 54 mg yellow powder (yield 67%); mp 180-182° C.; ¹H NMR (DMSO-d6): δ 13.20 (bs, 1H, COOH, D₂O exchange), 8.07 (d, 2H, carboxyphenyl, J=8.31 Hz), 7.80-8.00 (m, 5H, trifluoromethyl-phenyl and CH), 7.78 (d, 2H, carboxyphenyl, J=8.2 Hz); MS (ES⁻) (m/z): [M−1]⁻ calculated for C₁₈H₉F₃NO₃S₂, 408.40. found 408.23.

### Example 3

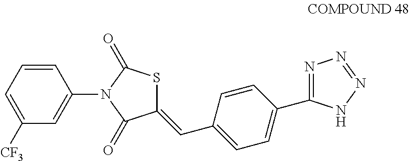

Synthesis of compound 48 was performed essentially as described in Example 1.

### Example 4

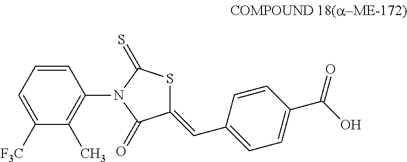

Synthesis of Compound 18 was performed as described in Example 1. See Scheme 1.

4-[[4-Oxo-2-thioxo-3-[2-methyl-3-(trifluoromethyl)phenyl]-5-thiazolidinylidene]methyl]benzoic acid (α-Me-172, 18): mp 156-158° C.; ¹H NMR (DMSO-d6): δ 12.74 (bs, 1H, COOH, D₂O exchange), 8.05 (d, 2H, carboxyphenyl, J=8.301 Hz), 7.906 (1H, s, ═CH—), 7.85 (d, 1H, J=7.813 Hz, trifluoromethylphenyl), 7.796 (d, 2H, J=8.301, carboxyphenyl), 7.739 (d, 1H, J=7.324 Hz, trifluoromethylphenyl), 7.585 (t, 1H, J=7.813 Hz, trifluoromethylphenyl), 2.132 (s, 3H, CH₃); MS (ES⁻) (m/z): [M−1]⁻ calculated for C₁₉H₁ₗF₃NO₃S₂, 422.43. found 422.34.

### Example 5

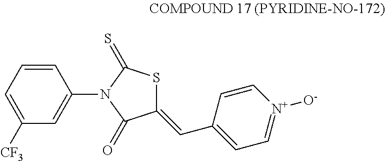

Synthesis of 5-(1-Oxido-4-pyridinyl)methylene)-2-thioxo-3-[3-(trifluoromethyl)phenyl]-4-thiazolidinone: A mixture of 2-thioxo-3-(3-trifluoromethyl phenyl)-4-thiazolidinone (55 mg, 0.2 mM, synthesized according to Sonawane et al., J. Pharm. Sci. 94, 134-143 (2005)), 4-pyridinecarboxaldehyde-1-oxide (25 mg, 0.2 mM), and sodium acetate (10 mg) in glacial acetic acid (0.5 ml) was refluxed for 8 h. Solvent was evaporated, residue crystallized from ethanol and resultant product was further purified by normal phase flash chromatography to yield 22 mg yellow powder (yield 29%); mp: 209-210° C. (decomp); MS (ES+) (m/z): [M+H]⁺ calculated for C₁₆H₉F₃N₂O₂S₂, 382.39. found 383.01. 5-[(1-Oxido-4-pyridinyl)methylene]-2-thioxo-3-[3-(trifluoromethyl)phenyl]-4-thiazolidinone (17): mp>200° C. (decomposition); MS (ES⁺) (m/z): [M+1]⁺ calculated for C₁₆H₉F₃N₂O₂S₂, 383.40. found 383.08.

### Example 6

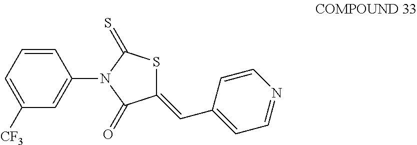

5-(4-Pyridinylmethylene)-2-thioxo-3-[3-(trifluoromethyl)phenyl]-4-thiazolidinone (33): mp 186-188° C.; ¹H NMR (DMSO-d6): δ 8.72 (dd, 2H, J=6.348, 2.930 Hz, pyridine), 7.918 (s, 1H, ═CH—), 7.869 (d, 1H, J=7.324 Hz, trifluoromethylphenyl), 7.811-7.748 (m, 3H, trifluoromethylphenyl), 7.595 (dd, 2H, J=6.348, 2.930 Hz, pyridine); MS (ES⁺) (m/z): [M+1]⁺ calculated for C₁₆H₉F₃N₂OS₂, 367.40. found 367.20.

### Example 7

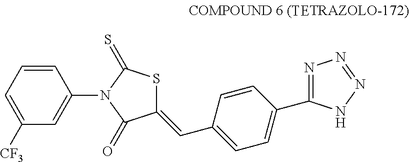

Synthesis of the tetrazolo group was performed essentially as described (see, e.g., Rostovtsev et al., Angew. Chem., Int. Ed. 41:2596-2599 (2002)). 1-Ethynyl-3-(trifluoromethyl)-benzen (0.85 g, 5 mmol) and 4-azidobenzoic acid (0.815 g, 5 mmol) were suspended in a 1:1 mixture of water and tert-butyl alcohol (10 mL). Freshly prepared solution of sodium ascorbate (0.3 mmol, 300 μL of 1 M) was added, followed by copper(II) sulfate pentahydrate (7.5 mg, 0.03 mmol, in 100 μL, of water). The mixture was stirred for 24 hr at room temperature until analysis by LC/MS indicated completion of reaction. The reaction mixture was diluted with water, white precipitate collected by filtration, washed and dried. After washing the precipitate with cold water (2×25 mL), the precipitate was dried under vacuum to afford 1.53 g (92%) of pure product as an off-white powder.

Compound 6 was synthesized as described in Example 1. 5-[[4-(2H-Tetrazol-5-yl)phenyl]methylene]-2-thioxo-3-[3-(trifluoromethyl)phenyl]-4-Thiazolidinone (Tetrazolo-172, 6): mp 216-219° C.; ¹H NMR (DMSO-d6): δ 11.92 (bs, 1H, tetrazolo-H, D2O exchange), 8.16 (d, 2H, carboxyphenyl, J=8.31 Hz), 8.026 (s, 1H), 7.92 (s, 1H), 7.87-7.84 (m, 3H, carboxyphenyl or trifluoromethyl-phenyl and/or CH), 7.79-7.76 (m, 2H, trifluoromethylphenyl); MS (ES⁻) (m/z): [M−1]⁻ calculated for C₁₈H₉F₃N₅OS₂, 432.43. found 432.49.

### Example 8

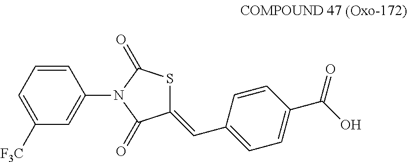

Compound 47 was prepared as described in Example 1. Oxo-172 47 was synthesized by condensation of 2,4-thiazolidinedione intermediate 3 with 4-carboxybenzaldehyde (Scheme 1). The isocyanate (Route 2) was used for the synthesis of intermediate 3. 4-[[3-[3-(trifluoromethyl)phenyl]-2,4-dioxo-5-thiazolidinylidene]methyl]benzoic acid (Oxo-172, 47): mp 168-170° C.; ¹H nmr (DMSO-d6): δ 13.054 (bS, 1H, COOH, D₂O exchange), 8.057-8.036 (d, 2H, carboxyphenyl, J=8.30 Hz), 8.011 (s, 1H), 7.916 (s, 1H), 7.860-7.842 (m, 1H), 7.778-7.743 (m, 4H); MS (ES⁻) (m/z): [M−1]⁻ calculated for C₁₈H₁₀F₃NO₄S, 392.34. found 392.18.

### Example 9

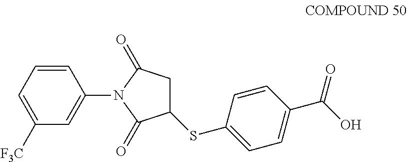

4-[[2,5-dioxo-1-[3-(trifluoromethyl)phenyl]-3-pyrrolidinyl]thio]-benzoic acid (50). 3-Trifluoromethylaniline was reacted with dichloromaleic anhydride or maleic anhydride in refluxing acetic anhydride to yield maleimide 4a and 4b analogs, respectively (Scheme 1). Subsequent reaction with 4-mercaptobenzoic acid produced 50, (Scheme 1, dotted line indicate double bond in Compound 49). MS (ES⁻) (m/z): [M−1]⁻ calculated for C₁₈H₁₂F₃NO₄S, 394.35. found 394.18.

Compound 50 had moderate CFTR inhibitory activity (IC₅₀ ˜7 μM) (see Table 2).

### Example 10

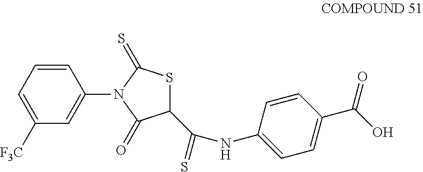

4-Oxo-[(3-trifluoromethyl)phenyl]-2-thioxo-N-[4-(carboxy)phenyl]-5-thiazolidinethiocarboxamide (51). Aryl isothiocyanates were reacted with 3 in the present of base DBU at room temperature (see Scheme 1 in Example 1). MS (ES⁺) (m/z): [M+1]⁺ calculated for C₁₈H₁₁F₃N₂O₃S₃, 457.49. found 457.37.

### Example 11

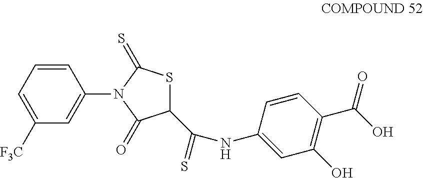

4-oxo-[(3-trifluoromethyl)phenyl]-2-thioxo-N-[(4-carboxy-2-hydroxy)phenyl]-5-thiazolidinethiocarboxamide (52): MS (ES⁺) (m/z): [M+1]⁺ calculated for C₁₈H₁₁F₃N₂O₄S₃, 473.50. found 473.23. (See Example 1).

### Example 12

### General Synthesis Schemes 2-4

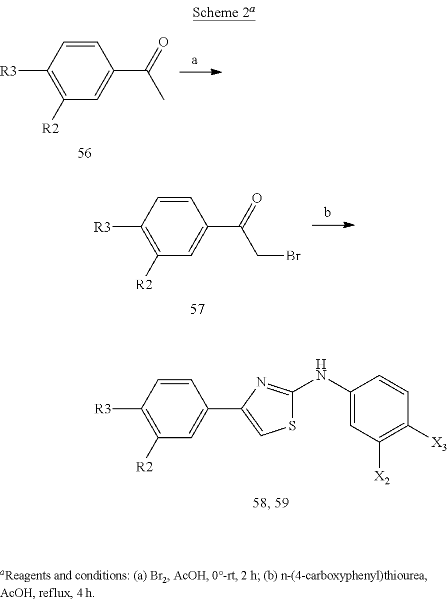

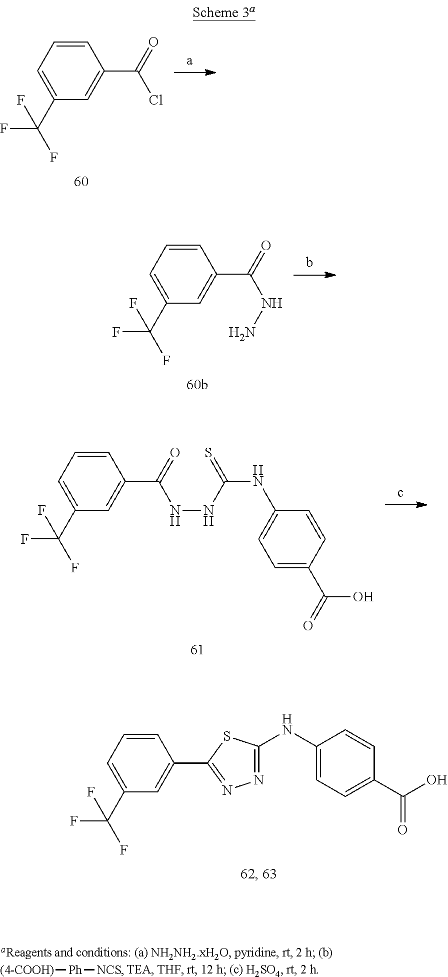

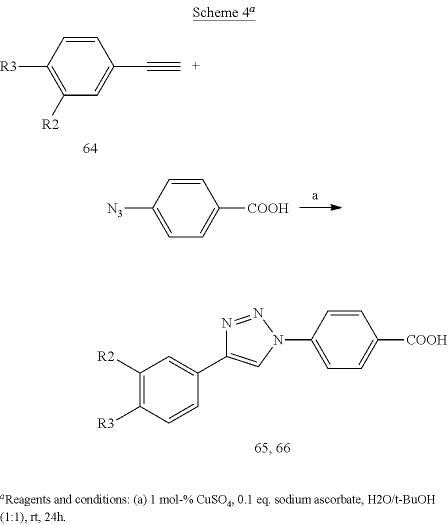

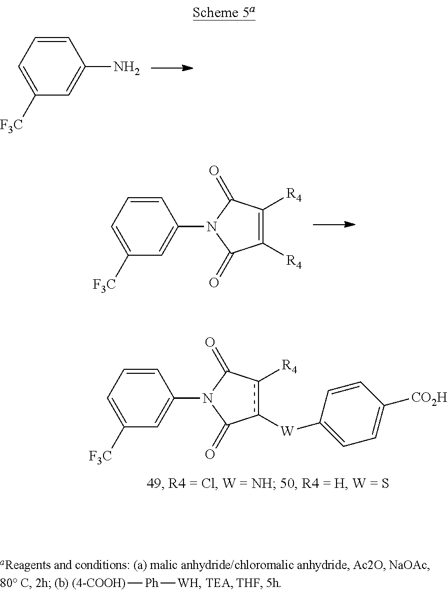

Synthesis Scheme 2 describes synthesis of compounds that instead of a thiazolidinone group as Ring B (see FIG. 1), have a thiazole, thiadiazole, or 1,2,3-triazole group. See also Table 2. Thiadiazole synthesis is performed by methods known in the art, for example, Oruc et al., J. Med. Chem. 47:6760-67 (2004). As shown in Scheme 2, thiazole analogs 59 and 60 were synthesized by bromination of acetophenone 57 in acetic acid at 0° C. for 2 h, followed by reaction with substituted phenylthiourea in refluxing ethanol.

Synthesis of thiadiazole compounds 64 and 65 proceeded as shown in Scheme 3 and as outlined below.

Synthesis of compound 62: 3-Trifluoromethyl benzoic Acid Hydrazide 60b: Hydrazine hydrate (4 equivalent) was added to the stirred solution of 3-trifluoromethyl benzoyl chloride 60 in pyridine at 0° C. Reaction mixture was added to the ice cold water and precipitate was collected and recrystallized from ethanol.

Thiosemicarbazides 61. Mixture of above hydrazide 60b (1 g, 5 mmol) and appropriate carboxyphenylisothiocyanate (5 mmol) in THF was refluxed for 2 hrs. Solid, obtained after solvent evaporation, was purified by recrystallization from ethanol to afford thiosemicarbazide 61 (yield 74-84%).

4-[(5-(3-Trifluoromethylphenyl)-1,3,4-thiadiazol-2-yl)amino]-benzoic acid (Compound 62). Thiosemicarbazide 61 (2.6 mmol) was added portion wise to 10 ml of concentrated sulfuric acid; stirred for 30 min at room temperature and reaction content was slowly dumped into stirring ice-water mixture. The precipitated product was purified by flash chromatography to yield (77%) of final product, 62. ¹H NMR (DMSO-d6): δ10.985 (bs, 1H, COOH), 8.136-8.112 (m, 3H, trifluoromethylphenyl), 7.904 (d, 2H, J=8.301 Hz, carboxyphenyl) 7.845-7.712 (m, 5H, carboxyphenyl and trifluoromethylphenyl, NH); MS (ES⁺) (m/z): [M+1]⁺ calculated for C₁₆H₁₀F₃N₃O₂S, 366.35. found 366.46.

Scheme 4 shows the synthesis of 1,2,3-triazoles. 1,3-Dipolar cycloaddition of alkynes 66 and 67 with 4-azidobenzoic acid produced the 1,2,3-triazoles (compounds 68 and 69) in high yields. Single spot in TLC indicated that adducts were predominantly 4-regioisomers.

Scheme 5 shows synthesis of compounds 49 and 50. Briefly, maleimide intermediates 4 (R4=Cl or H) were prepared by reaction of 3-trifluoromethylaniline with dichloromaleic anhydride (R4=Cl) or maleic anhydride (R4=H) in refluxing acetic anhydride (Scheme 2). Subsequent reaction with 4-aminobenzoic acid and 4-mercaptobenzoic acid produced compounds 49 and 50 (dotted line indicates double bond in 49).

### Example 13

### Synthesis of an Inactive Analog of CFTRᵢₙₕ-172

Synthesis of inactive-CFTRᵢₙₕ-172: 5-(N,N-dimethylphenyl)methylene)-2-thioxo-3-[3-(trifluoromethyl)phenyl]-4-thiazolidinone: A mixture of 2-thioxo-3-(3-trifluoromethyl phenyl)-4-thiazolidinone (110 mg, 0.4 mM), 4-(N,N-dimethyl)benzaldehyde (59 mg, 0.4 mM), and sodium acetate (50 mg) in glacial acetic acid (1 ml) was refluxed for 4 h. Solvent was evaporated, residue dissolved in ethyl acetate, filtered, and silica gel (1 g) was added. Compound was purified on silica gel by normal phase flash chromatography to yield 68 mg yellow-orange crystals (yield 42%); mp: 224-226° C.; MS (ES+) (m/z): [M+H]⁺ calculated for C₁₉H₁₅F₃N₂OS₂, 409.4. found 409.3.

### Example 14

### CFTR Inhibitory Activity of Thiazolidinone Derivative Compounds

Fluorescence cell-based assay of CFTR inhibition. CFTR inhibition by thiazolidinone derivative compounds was determined by a fluorescence cell-based assay utilizing doubly transfected cells expressing human wild-type CFTR and a yellow fluorescent protein (YFP) iodide sensor, as described (see, e.g., Galietta, et al., J. Physiol. 281:C1734-C1742 (2001)). Fisher rat thyroid (FRT) cells stably expressing wild-type human CFTR and YFP-H148Q were cultured on 96-well black-wall plates as described (see, e.g., Ma, et al., J. Clin. Invest. 110:1651-1658 (2002)). Cells in 96-well plates were washed three times, and then CFTR was activated by incubation for 15 minutes with an activating cocktail containing 10 μM forskolin, 20 μM apigenin, and 100 μM IBMX. Test compounds were added 5 minutes before assay of iodide influx in which cells were exposed to a 100 mM inwardly directed iodide gradient. YFP fluorescence was recorded for 2 seconds before and 12 seconds after creation of the iodide gradient. Initial rates of iodide influx were computed from the time course of decreasing fluorescence after the iodide gradient.

CFTR-facilitated iodide influx following extracellular iodide addition results in quenching of cytoplasmic YFP fluorescence. IC₅₀ data are presented in Tables 1 and 2.

TABLE 1
CFTR Inhibitory Activity of Thiazolidinone Compounds Having Structure I

| Cmpd | R₃ | R₂ | R₁ | R₉ | X₁ | X₂ | X₃ | X₄ | IC₅₀ |
|---|---|---|---|---|---|---|---|---|---|
| 5 | H | CF₃ | H | H | H | H | COOH | H | 0.1-1 |
| (CFTRᵢₙₕ- | | | | | | | | | |
| 172) | | | | | | | | | |
| 6 | H | CF₃ | H | H | H | H | tetrazolo-5-yl | H | 0.1-1 |
| (Tetrazolo- | | | | | | | | | |
| 172) | | | | | | | | | |
| 7 | H | CF₃ | H | H | H | COOH | OH | H | 1-2 |
| 8 | F | CF₃ | H | H | H | H | COOH | H | 1-2 |
| 9 | H | CF₃ | H | H | H | COOH | H | H | 1-2 |
| 10 | H | CF₃ | H | H | H | H | O—CH₂—COOH | H | 2-3 |
| 11 | F | CF₃ | H | H | H | COOH | H | H | 5-10 |
| 12 | H | CH₃ | H | H | H | H | COOH | H | 5-10 |
| 13 | H | CH₃ | CH₃ | H | H | H | COOH | H | 5-10 |
| 14 | H | CF₃ | H | H | H | Br | OH | Br | 5-10 |
| 15 | H | CF₃ | H | H | OH | Br | OH | Br | 5-10 |
| 16 | H | CF₃ | H | H | OH | H | COOH | H | 7-9 |
| 18 | H | CF₃ | CH₃ | H | H | H | COOH | H | 5-10 |
| (α-Me- | | | | | | | | | |
| 172) | | | | | | | | | |
| 19 | H | CF₃ | H | H | H | OH | OH | OH | 10-20 |
| 20 | H | H | CF₃ | H | H | H | COOH | H | 10-20 |
| 21 | CF₃ | H | H | H | H | COOH | H | H | 10-20 |
| 22 | H | H | CF₃ | H | H | COOH | OH | H | 10-20 |
| 23 | H | CF₃ | H | CF₃ | H | H | COOH | H | 10-20 |
| 24 | Cl | CF₃ | H | H | H | H | COOH | H | 10-20 |
| 25 | CF₃ | H | H | H | H | H | COOH | H | 10-20 |
| 26 | H | CF₃ | H | H | COOH | H | H | H | 10-20 |
| 27 | H | H | CF₃ | H | COOH | H | H | H | 10-20 |
| 28 | H | H | CF₃ | H | H | COOH | H | H | 10-20 |
| 29 | CF₃ | H | H | H | COOH | H | H | H | 10-20 |
| 30 | Cl | H | H | CF₃ | H | H | COOH | H | 10-20 |
| 31 | H | CF₃ | H | H | H | H | OH | H | 20-30 |
| 32 | CF₃ | H | H | H | H | COOH | OH | H | 20-30 |
| 34 | H | CF₃ | H | H | OH | OH | OH | H | 20-30 |
| 35 | H | CF₃ | H | H | H | H | SO₃Na | H | 20-30 |
| 36 | H | CF₃ | H | H | H | O—CH₂—COOH | H | H | 20-30 |

| | |
|---|---|
| 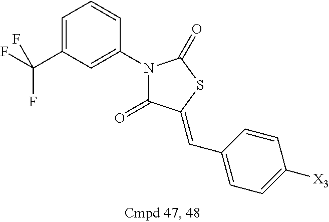 | 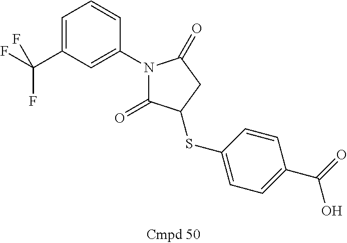 |

| Compound | X₃ | IC₅₀ (μM) |
|---|---|---|
| 47 (Oxo-172) | COOH | 1-2 |
| 48 | (2H)-Tetrazolo-5-yl | 10-20 |
| 50 | — | 5-10 |

| 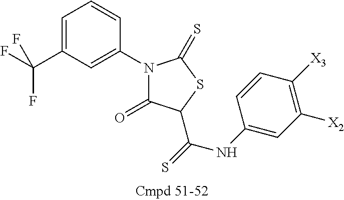 | 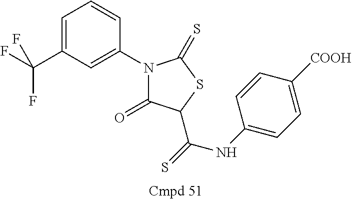 |
|---|---|
| Compound | X₃ |
| 51 | COOH |
| 52 | COOH |

| 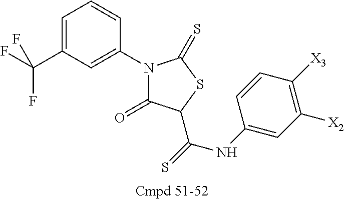 | 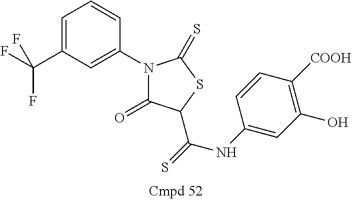 | |
|---|---|---|
| Compound | X₂ | IC₅₀ (μM) |
| 51 | H | 5-10 |
| 52 | OH | 10-20 |

Compounds of Structure I include compounds 17 and 33, which have the following structures and IC₅₀ values:

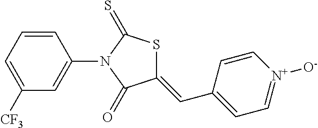

Compound 17; IC₅₀=5-10 μM (also referred to herein as Pyridine-NO-172)

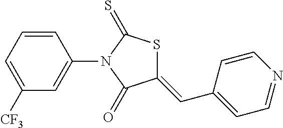

Compound 33; IC₅₀=20-30 μM (also referred to herein as T16).

TABLE 2
Summary Table

| # | Structure | Name^{a} | IC₅₀ |
|---|---|---|---|
| 5 | 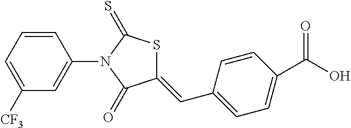 | (Z)-4-((4-oxo-2-thioxo- 3-(3-(trifluoromethyl) phenyl) thiazolidin-5- ylidene)methyl)benzoic acid | 0.1-1 |
| 6 | 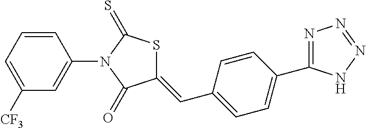 | (Z)-5-(4-(1H-tetrazol- 5-yl)benzylidene)- 2-thioxo-3-(3- (trifluoromethyl) phenyl)thiazolidin-4- one | 0.1-1 |
| 7 | 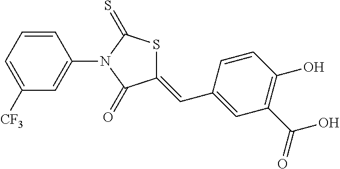 | (Z)-2-hydroxy-5-((4- oxo-2-thioxo-3- (3-(trifluoromethyl) phenyl) thiazolidin-5- ylidene)methyl)benzoic acid | 1-2 |
| 8 | 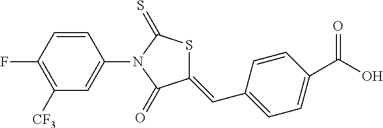 | (Z)-4-((3-(4-fluoro-3- (trifluoromethyl) phenyl)-4-oxo-2- thioxothiazolidin-5 ylidene)methyl)benzoic acid | 1-2 |
| 9 | 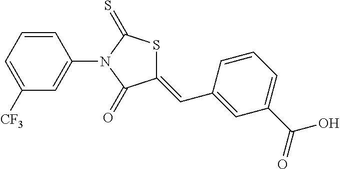 | (Z)-3-((4-oxo-2-thioxo- 3-(3-(trifluoromethyl) phenyl) thiazolidin-5- ylidene)methyl)benzoic acid | 1-2 |
| 10 | 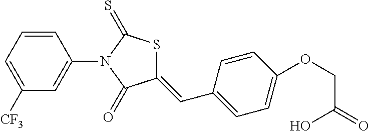 | (Z)-2-(4-((4-oxo-2- thioxo-3-(3- (trifluoromethyl) phenyl) thiazolidin-5- ylidene)methyl)phenoxy) acetic acid | 2-3 |
| 11 | 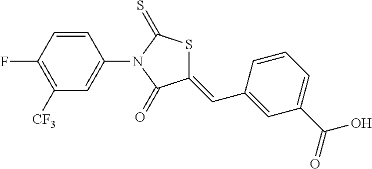 | (Z)-3-((3-(4-fluoro-3- (trifluoromethyl) phenyl)-4-oxo-2- thioxothiazolidin-5- ylidene)methyl)benzoic acid | 5-10 |
| 12 | 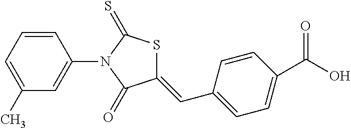 | (Z)-4-((4-oxo-2-thioxo- 3-m-tolylthiazolidin-5- ylidene)methyl)benzoic acid | 5-10 |
| 13 | 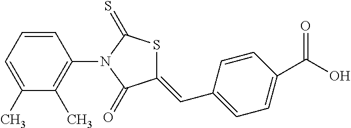 | (Z)-4-((3-(2,3- dimethylphenyl)-4- oxo-2- thioxothiazolidin-5- ylidene)methyl)benzoic acid | 5-10 |
| 14 | 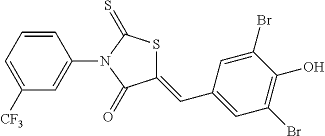 | (Z)-5-(3,5-dibromo-4- hydroxybenzylidene)- 2-thioxo-3- (3-(trifluoromethyl) phenyl) thiazolidin-4- one | 5-10 |
| 15 | 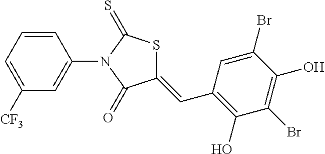 | (Z)-5-(3,5-dibromo- 2,4- dihydroxybenzylidene)- 2-thioxo-3- (3-(trifluoromethyl) phenyl) thiazolidin-4- one | 5-10 |
| 16 | 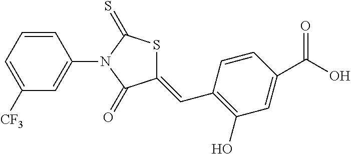 | (Z)-3-hydroxy-4-((4- oxo-2-thioxo-3- (3-(trifluoromethyl) phenyl) thiazolidin-5- ylidene)methyl)benzoic acid | 7-9 |
| 17 | 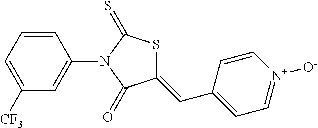 | (Z)-4-((4-oxo-2-thioxo- 3-(3-(trifluoromethyl) phenyl)thiazolidin-5- ylidene)methyl)pyridine 1-oxide | 5-10 |
| 18 | 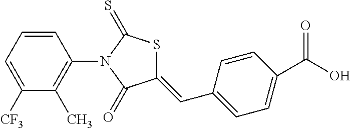 | (Z)-4-((3-(2-methyl-3- (trifluoromethyl) phenyl)-4-oxo-2- thioxothiazolidin-5- ylidene)methyl)benzoic acid | 5-10 |
| 19 | 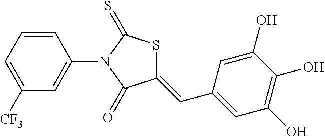 | (Z)-2-thioxo-3-(3- (trifluoromethyl) phenyl)-5-(3,4,5- trihydroxybenzylidene) thiazolidin-4-one | 10-20 |
| 20 | 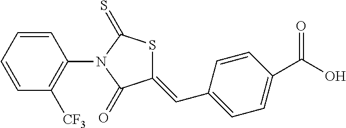 | (Z)-4-((4-oxo-2-thioxo- 3-(2-(trifluoromethyl) phenyl) thiazolidin-5- ylidene)methyl)benzoic acid | 10-20 |
| 21 | 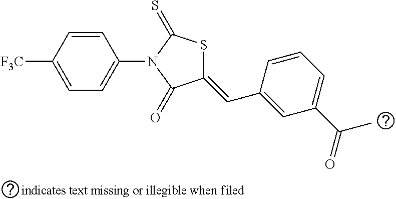 | (Z)-3-((4-oxo-2-thioxo- 3-(4-(trifluoromethyl) phenyl) thiazolidin-5- ylidene)methyl)benzoic acid | 10-20 |
| 22 | 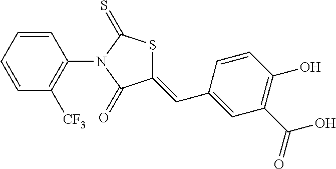 | (Z)-2-hydroxy-5-((4- oxo-2-thioxo-3-(2- (trifluoromethyl) phenyl) thiazolidin-5- ylidene)methyl)benzoic acid | 10-20 |
| 23 | 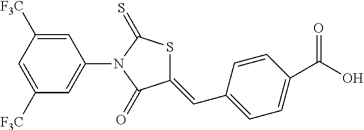 | (Z)-4-((3-(3,5- bis(trifluoromethyl) phenyl)-4-oxo-2- thioxothiazolidin-5- ylidene)methyl)benzoic acid | 10-20 |
| 24 | 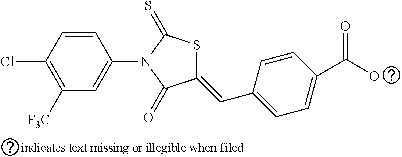 | (Z)-4-((3-(4-chloro-3- (trifluoromethyl) phenyl)-4-oxo-2- thioxothiazolidin-5- ylidene)methyl)benzoic acid | 10-20 |
| 25 | 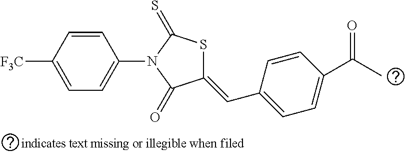 | (Z)-4-((4-oxo-2-thioxo- 3-(4-(trifluoromethyl) phenyl) thiazolidin-5- ylidene)methyl)benzoic acid | 10-20 |
| 26 | 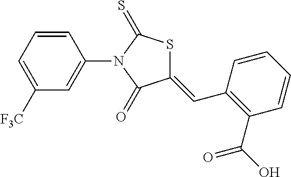 | (Z)-2-((4-oxo-2-thioxo- 3-(3-(trifluoromethyl) phenyl) thiazolidin-5- ylidene)methyl)benzoic acid | 10-20 |
| 27 | 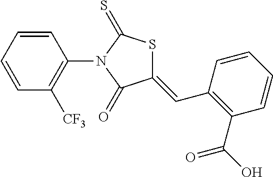 | (Z)-2-((4-oxo-2-thioxo- 3-(2-(trifluoromethyl) phenyl) thiazolidin-5- ylidene)methyl)benzoic acid | 10-20 |
| 28 | 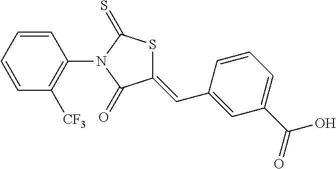 | (Z)-3-((4-oxo-2-thioxo- 3-(2-(trifluoromethyl) phenyl) thiazolidin-5- ylidene)methyl)benzoic acid | 10-20 |
| 29 | 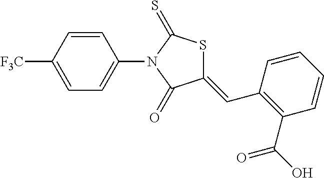 | (Z)-2-((4-oxo-2-thioxo- 3-(4-(trifluoromethyl) phenyl) thiazolidin-5- ylidene)methyl)benzoic acid | 10-20 |
| 30 | 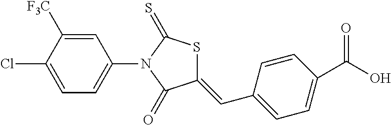 | (Z)-4-((3-(4-chloro-3- (trifluoromethyl) phenyl)-4-oxo-2- thioxothiazolidin-5- ylidene)methyl)benzoic acid | 10-20 |
| 31 | 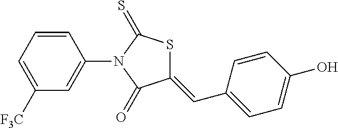 | (Z)-5-(4- hydroxybenzylidene)- 2-thioxo-3-(3- (trifluoromethyl) phenyl) thiazolidin-4- one | 20-30 |
| 32 | 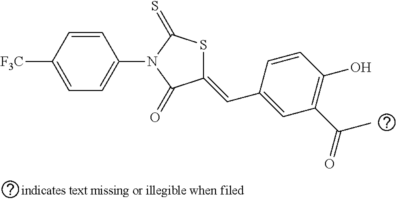 | (Z)-2-hydroxy-5-((4- oxo-2-thioxo-3-(4- (trifluoromethyl) phenyl) thiazolidin-5- ylidene)methyl)benzoic acid | 20-30 |
| 33 | 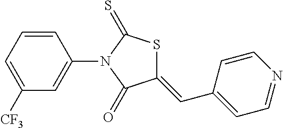 | (Z)-5-(pyridin-4- ylmethylene)-2-thioxo- 3-(3-(trifluoromethyl) phenyl) thiazolidin-4- one | 20-30 |
| 34 | 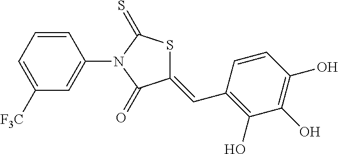 | (Z)-2-thioxo-3-(3- (trifluoromethyl) phenyl)-5-(2,3,4- trihydroxybenzylidene) thiazolidin-4-one | 20-30 |
| 35 | 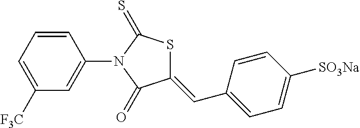 | sodium (Z)-4-((4-oxo- 2-thioxo-3-(3- (trifluoromethyl) phenyl) thiazolidin-5- ylidene)methyl) benzenesulfonate | 20-30 |
| 36 | 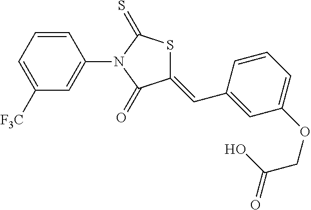 | (Z)-2-(3-((4-oxo-2- thioxo-3-(3- (trifluoromethyl) phenyl) thiazolidin-5- ylidene)methyl)phenoxy) acetic acid | 20-30 |
| 47 | 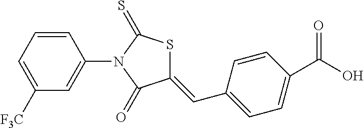 | (Z)-4-((2,4-dioxo-3-(3- (trifluoromethyl) phenyl) thiazolidin-5- ylidene)methyl)benzoic acid | 1-2 |
| 48 | 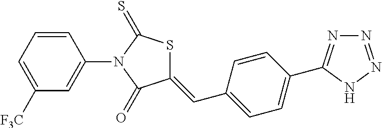 | (Z)-5-(4-(1H-tetrazol- 5-yl)benzylidene)-3-(3- (trifluoromethyl) phenyl) thiazolidine- 2,4-dione | 10-20 |
| 50 | 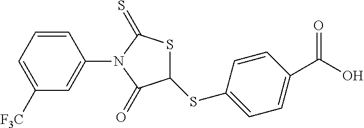 | 4-(2,5-dioxo-1-(3- (trifluoromethyl) phenyl) pyrrolidin-3- ylthio)benzoic acid | 5-10 |
| 51 | 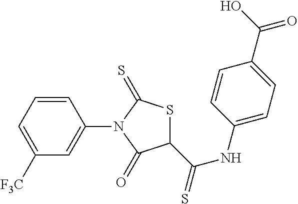 | 4-(4-oxo-2-thioxo-3-(3- (trifluoromethyl) phenyl) thiazolidine-5- carbothioamido)benzoic acid | 5-10 |
| 52 | 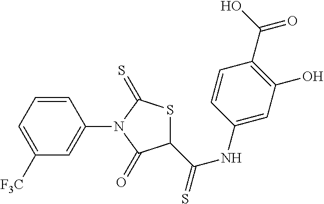 | 2-hydroxy-4-(4-oxo-2- thioxo-3-(3- (trifluoromethyl) phenyl) thiazolidine-5- carbothioamido)benzoic acid | 10-20 |

^{a}Naming of structures was performed using naming IUPAC conventions available in CambridgeSoft ® Chem & Bio Draw 11.0 (CambridgeSoft ® Corporation, Boston, MA). Even though the naming convention indicates the Z-isoform of the thiazolidinone compound structures, as described herein, the compounds may exist in either the E or Z geometric isoform.

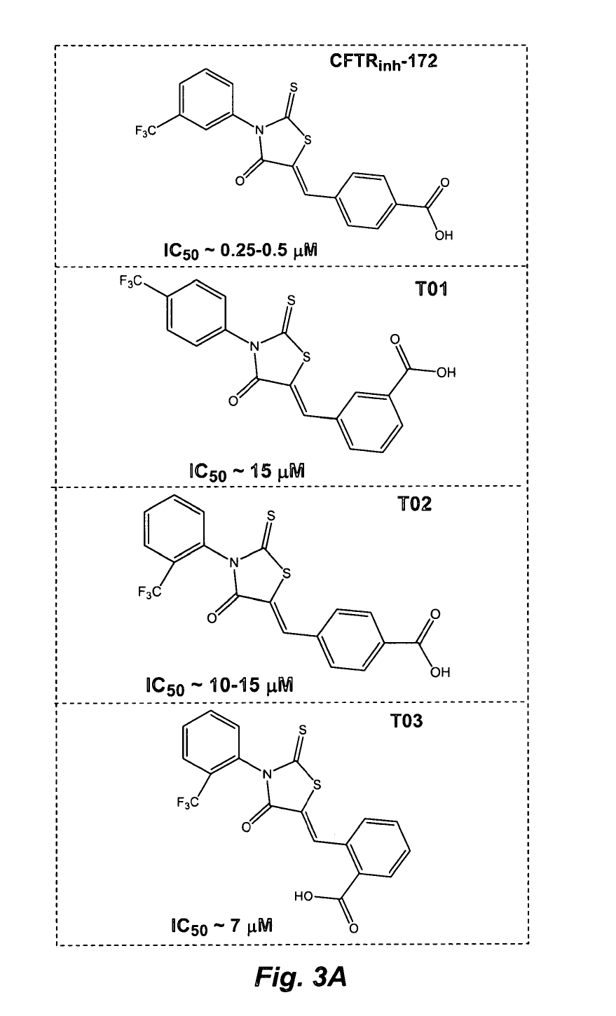

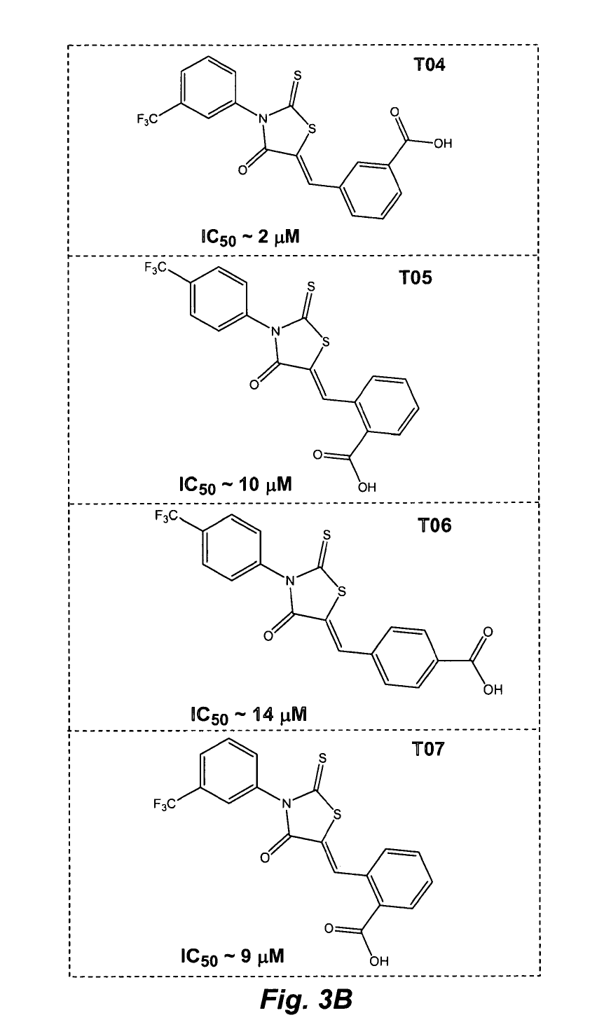

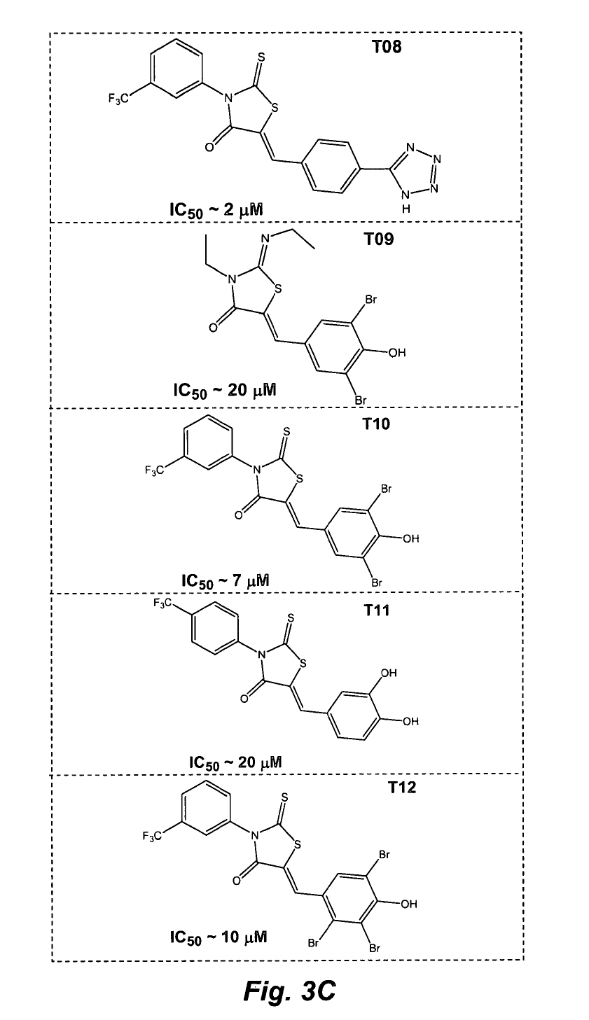

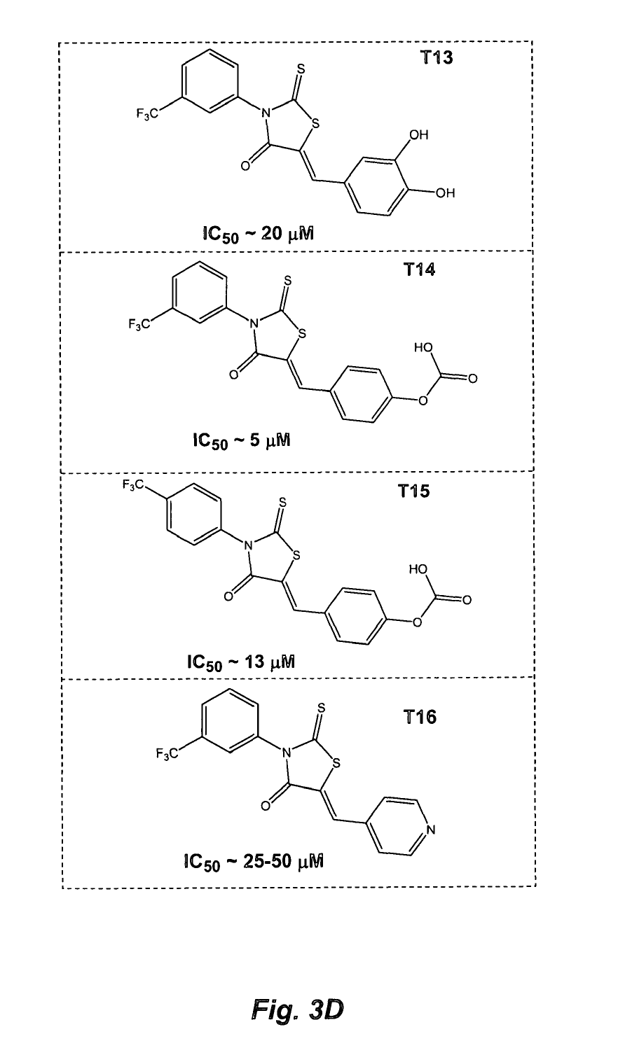

Some of the compounds provided in Table 2 are also referred to in FIG. 3 (see Example 20).

### Example 15

### Biological Methods

Solubility measurements. A saturated compound solution was prepared by addition of DMSO stock to phosphate buffered saline (final DMSO 2%) followed by sonication for 5 min at 25° C. and shaking at room temperature for 1 h. After centrifugation at 15,000 rpm for 1 h, the supernatant was analyzed by LC/MS with concentration determined from area under the curve, standardized against calibration data. A standard curve for each compound was obtained by plotting area under the curve from chromatograms against inhibitor concentration. The concentration range of the standard solutions was 1-15 μM (for 5) and 1-100 μM (other compounds). In all cases, standard curves prepared were linear with r>0.998.

Short-circuit current measurements. FRT cells (stably expressing human wildtype CFTR) were cultured on SNAPWELL filters with 1 cm² surface area (Corning-Costar) to resistance >1,000 Ω·cm² as described (see, e.g., Ma et al., J. Clin. Invest. 110:1651-1658 ((2002)); Sonawane et al., FASEB J. 20:130-132 (2006)). Filters were mounted in an Easymount Chamber System (Physiologic Instruments, San Diego). For apical current measurements, the basolateral hemichamber contained (in mM): 130 NaCl, 2.7 KCl, 1.5 KH₂PO₄, 1 CaCl₂, 0.5 MgCl₂, 10 Na-HEPES, 10 glucose (pH 7.3). The basolateral membrane was permeabilized with amphotericin B (250 μg/ml) for 30 min. In the apical solution 65 mM NaCl was replaced by sodium gluconate, and CaCl₂ was increased to 2 mM. Solutions were bubbled with 95% O₂/5% CO₂ and maintained at 37° C. Current was recorded using a DVC-1000 voltage-clamp (World Precision Instruments) using Ag/AgCl electrodes and 1 M KCl agar bridges.

Inhibitor absorption was performed as described (see, e.g., Sonawane, supra, 2006). For measurements of intestinal absorption, midjejunal loops were injected with 100 μl of phosphate buffered-saline containing 20 μM test compound and 5 μg FITC-dextran (40 kDa), in which 100 mM NaCl was replaced by 200 mM raffinose. The added raffinose prevented intestinal fluid absorption. After 0 or 2 h, loop fluid was withdrawn for assay of compound concentration from the ratio of optical absorbance of test compound vs. FITC-dextran (OD342/OD494 nm), which was assumed to be impermeant. In some experiments, fluid samples were also analyzed by LC/MS.

Cytotoxicity measurements. FRT cells in confluent monolayers were incubated with compounds for 2 days. Cells were washed 3 times, fixed (cytofix, 30 min) and stained with crystal violet (100 μl, 0.5%, 10 min) using standard procedures. Excess crystal violet was removed by washing, and dye was extracted with Sorenson's buffer (0.1 M sodium citrate, 50% ethanol, pH 4.2). Crystal violet was quantified by measurement of absorbance at 650 nm. The percentage crystal violet staining was determined from test wells measured 8 times, compared to blanks (wells not containing cells) and vehicle-treated cells.

### Example 16

### Solubility and Cytotoxicity of Thiazolidinone Derivative Compounds

The maximum solubility in saline of Compounds 5 (CFTRinh-172), 6 (tetrazolo-172), 18 (α-ME-172), 7, 9, 16, 17 (pyridine-NO-172), and 47 (Oxo-172) was determined. Results are shown in Table 3. A saturated drug solution of a compound was prepared by addition of the compound in DMSO to phosphate buffered saline (PBS), followed by sonication for 5 min at 25° C. Final concentration of DMSO was <2%. Each saturated solution was centrifuged and filtered. The saturated supernatant solution was diluted and injected into LC/MS. The concentration was determined from area under the curve using a calibration curve.

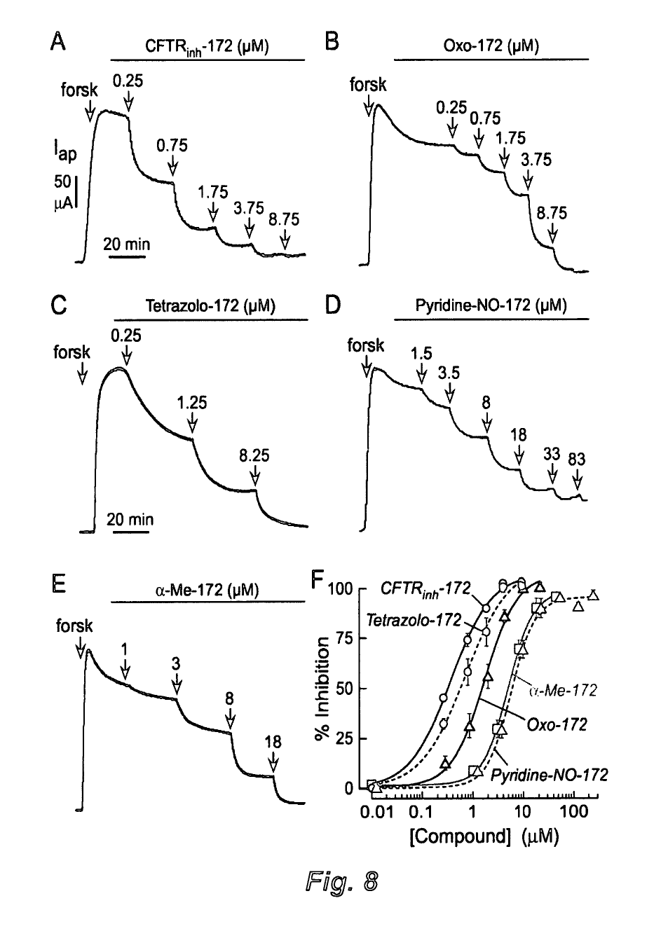

Introduction of methyl at the 2-position in α-Me-172, 18, increased water solubility to 259 μM, but reduced inhibition potency. FIG. 8 shows concentration-dependent inhibition of CFTR by CFTRᵢₙₕ-172 (A) and α-Me-172 (E) with IC₅₀ 0.4 and 8 μM, respectively. As determined by LC/MS, the solubility of α-Me-172 in saline was 259 μM, substantially greater than that of CFTRᵢₙₕ-172 (17 μM; Table 3). Addition of polar substituents such as hydroxy, SO₃Na or COOH in Ring A, or removal of CF₃, produced highly water-soluble though inactive compounds.

The thiazolidinone core, Ring B, was replaced by thiazolidinedione, maleimide, succinimide, thiazole, thiadiazole and triazole, while keeping Rings A and C and their substituents the same as in CFTRᵢₙₕ-172. The 2,4-thiazolidinedione 47 is a close analog of CFTRᵢₙₕ-172 in which the thioxo group is replaced by an oxo-group (referred to as Oxo-172). Replacement of 2-thioxo by 2-oxo increased solubility in saline by ˜25-fold, while reducing CFTR inhibition potency 3.6-fold in short-circuit current assays (FIGS. 8B, 8F; Table 1).

Cytotoxicity analysis was performed for Tetrazolo-172, Oxo-172, α-Me-172 and Pyridine-NO-172 (Compound 17) and compared with CFTRᵢₙₕ-172 (Table 3). Compounds were incubated with cell cultures for 48 h and cell viability determined by crystal violet staining Compounds at 20 μM showed little cytotoxicity, with staining approximately 90% of that of control (vehicle-treated cultures). Crystal violet staining was reduced for Tetrazolo-172 and α-Me-172 at 50 μM. CFTRᵢₙₕ-172 was not studied at 50 μM because of its limited aqueous solubility.

TABLE 3
Inhibition potency, solubility, and toxicity of CFTR inhibitors.

| | | Solubility | % Cell viability | |
|---|---|---|---|---|
| Compound | IC₅₀ (μM) | in saline (μM) | 20 μM | 50 μM |
| CFTRᵢₙₕ-172 | 0.38 ± 0.04 | 17 | 86 | not soluble |
| Tetrazolo-172 | 0.76 ± 0.2 | 189 | 87 | 73 |
| Oxo-172 | 1.4 ± 0.2 | 420 | 93 | 91 |
| α-Me-172 | 8.2 ± 0.4 | 259 | 89 | 72 |

| | | | |
|---|---|---|---|
| Pyridine-NO-172 | 8.7 ± 0.7 | 264 | not determined |

Maleimide analog 49 had weak inhibitory activity, though 2,5-pyrrolidinedione 50 had moderate activity with IC₅₀ ˜7 μM. The Ring B variation in 50 removed the double bond in CFTRᵢₙₕ-172, making it less reactive for Michael addition. The aminothiazole and aminothiadiazole analogs were inactive, however. Replacement of thiazolidinone by other heterocycles such as aminothiazoles (59, 60) gave weakly active compounds. Replacement of thiazolidinone by aminothiadiazole (64, 65), and 1,2,3-triazoles (68, 69) yielded inactive compounds.

C-ring substitutions were carried out in an attempt to increase compound solubility, and to convert compound net negative charge at physiological pH to neutral in order to increase compound accumulation in cytoplasm. Analogs were synthesized with different substitutions on Ring C, keeping Rings A and B, and both linkers the same as in CFTRᵢₙₕ-172. Substitutions were chosen to increase compound polarity and H-bond capacity, including carboxy, esters, amides, hydroxy, methoxy and sulfonate.

Monosubstituted compounds containing 4-COOH(CFTRᵢₙₕ-172) or 3-COOH, 9, showed greater CFTR inhibition potency than 2-COOH, 26. Esterification or amidation of 4-COOH in CFTRᵢₙₕ-172 gave inactive compounds 42-46. Compounds 31, 38, 19, and 34 containing mono, di or tri hydroxy functions at Ring C had low activity. Based on predicted pKa of >7.5, these compounds are expected to be neutral at physiological pH. Creation of a negative charge by addition of 3,4-dibromo electron withdrawing moieties, that lower the pKa of 4-OH, resulted in modest inhibition activity (compounds 14 and 15; Table 1), whereas modification of the ionizable 4-OH to 4-OMe yielded the inactive compound 39. However, sulfonic acid derivatives 35 and 37, which carry at physiological pH single and double negative charges, respectively, were inactive.

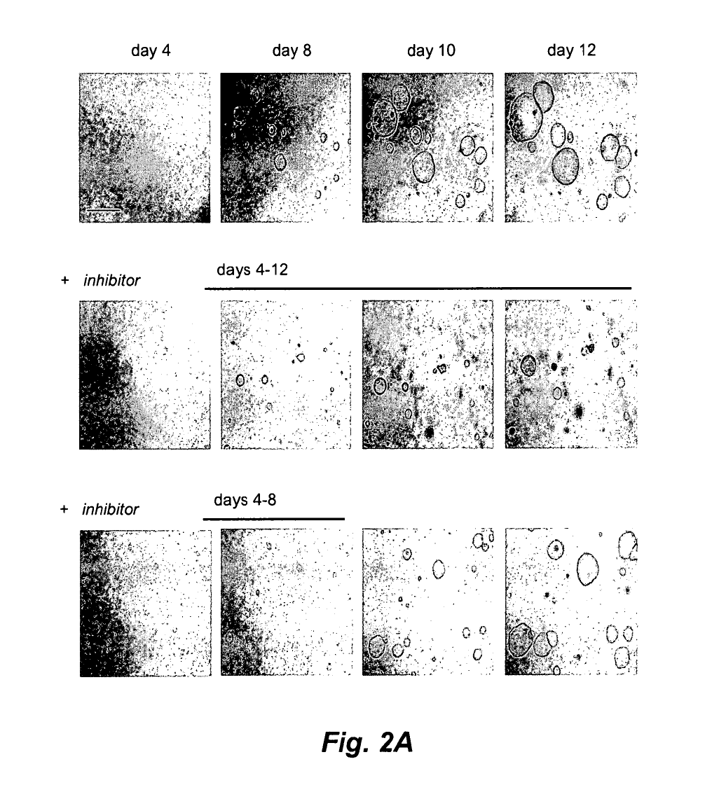

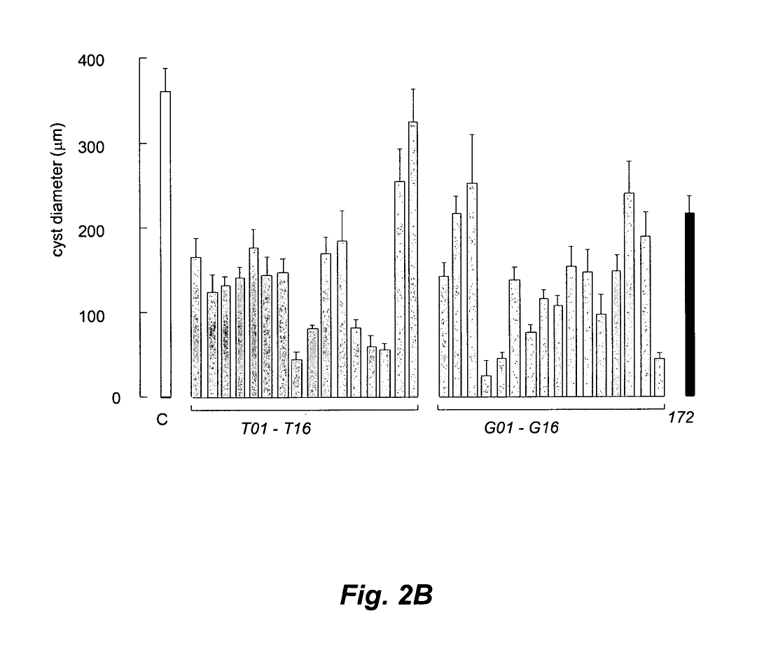

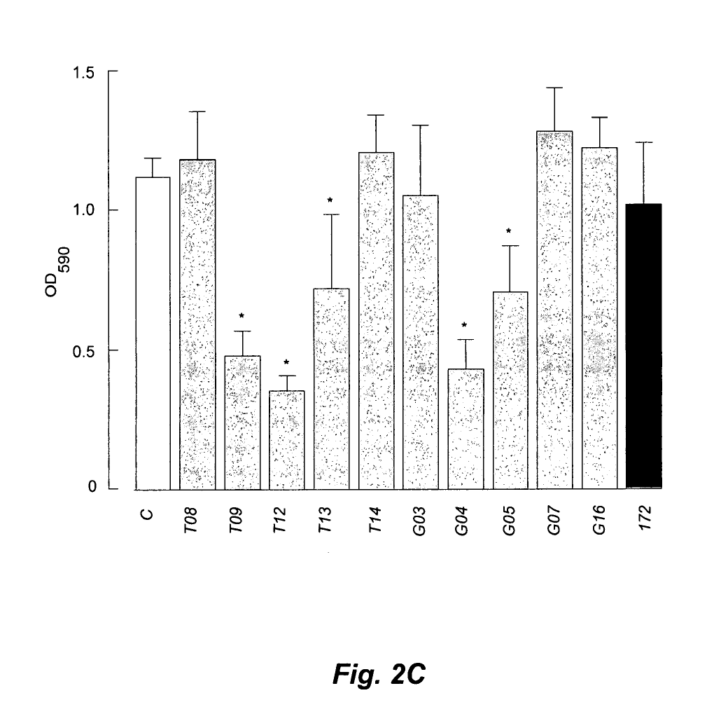

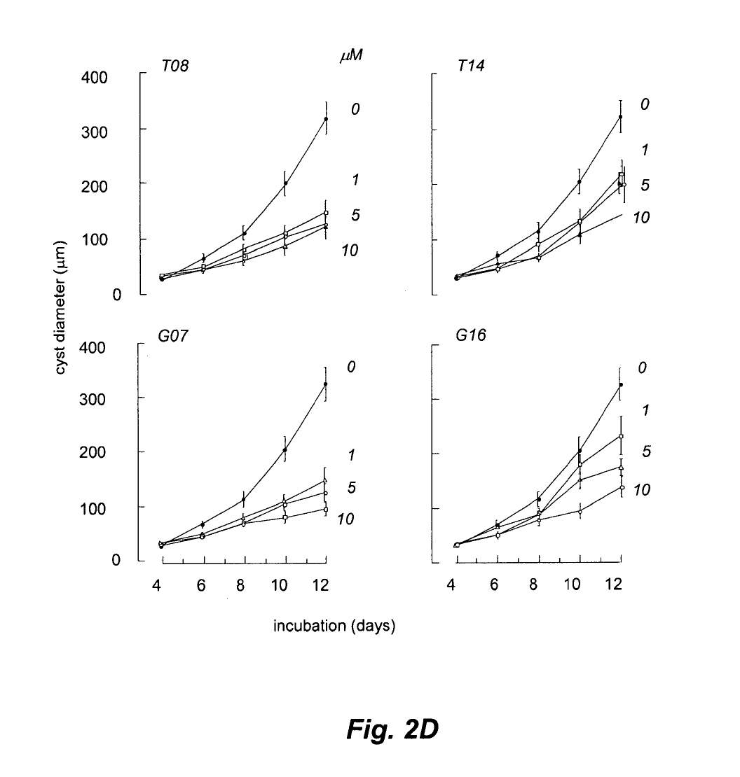

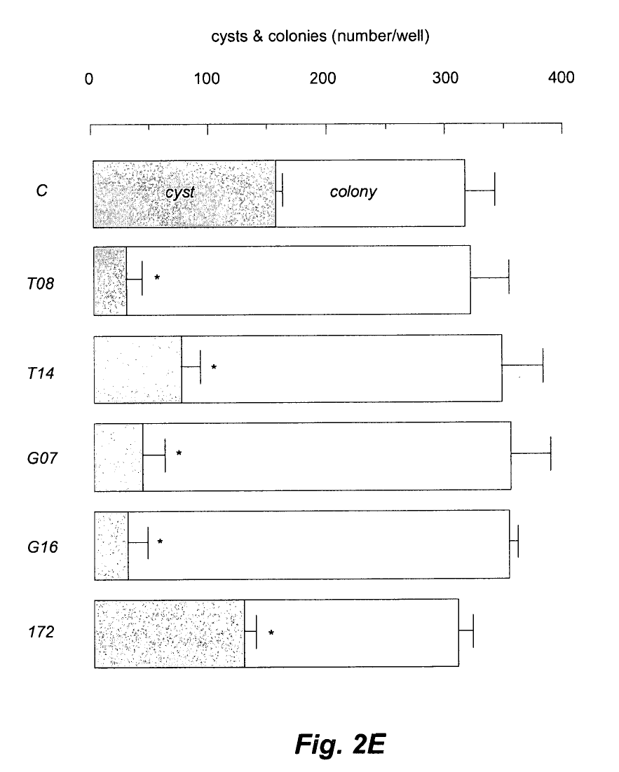

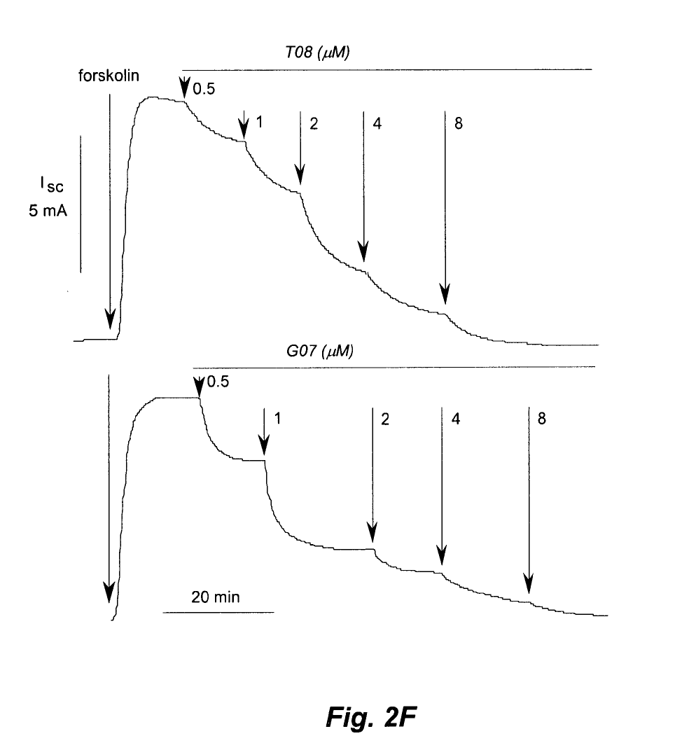

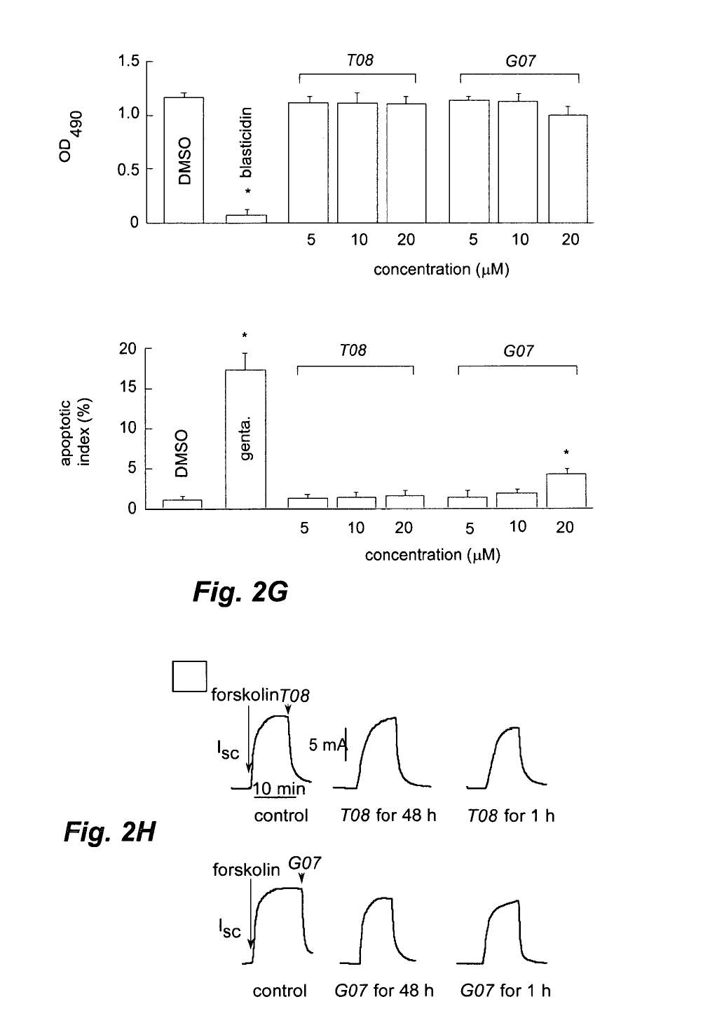

Ring C was also replaced by heterocyclic-equivalent ring systems. Replacement of the phenyl by a pyridyl ring gave the inactive neutral compound 33; however, N-oxidation of pyridyl nitrogen gave the first, net-neutral thiazolidinone CFTR inhibitor, pyridine-NO-172, 17, with IC₅₀ 9 μM (FIGS. 2D, 2F). This is a zwitterionic compound containing a positive charge on the ring nitrogen and negative charge on the oxygen. Addition of hetero atoms is expected to increase aqueous solubility by H-bonding and increased polarity. Pyridine-NO-172 had high aqueous solubility of 264 μM. Similarly, the polar analog 10 containing a 4-carboxymethoxy group had an IC₅₀ of 2.6 μM (Table 1). Moving the carboxymethoxy group from the 4-position (as in 10) to the 3-position (as in 36) reduced CFTR inhibition.

As another approach to improve water solubility, the 4-COOH in ring C was replaced by tetrazolo-5-yl, an isoster of carboxy with delocalized negative charge at physiological pH. The tetrazolo substitution increased water solubility 11-fold (Table 3) with IC₅₀ of 0.8 μM (FIG. 2F). As shown in FIG. 2C, Tetrazolo-172 showed slower inhibition kinetics than CFTRᵢₙₕ-172 or Oxo-172. Similarly replacement of carboxylate in 48 by tetrazolo in 49, gave lower inhibition potency (IC₅₀ 17 μM; Table 2).

CFTRᵢₙₕ-172 contains a single bond as a Linker 1 that directly connects lipophilic Ring A to heterocyclic Ring B. Introduction of a methylene group as a bridge between Rings A and B produced an inactive compound (Compound 47), however.

Because the double bond in Linker 2 is a Michael electrophile, alternative linkers between Ring B and Ring C were investigated. First, reduction of double bond linker of CFTRᵢₙₕ-172 gave compound 56, which was inactive. The double bond reduction disturbed the rigid geometry of CFTRᵢₙₕ-172, which is presumed as Z-configuration, allowing free rotation of Rings B and C. Further, replacement of the double bond-methylidyne bridge by thioamide in 52 and 53, though highly water soluble, were inactive, as were analogs 54 and 55 containing sulfonic acid substituents (Scheme 1 and Table 2).

### Example 17

### CFTR Inhibitory Activity of Thiazolidinone Compounds in Using Chamber

Short-circuit current measurements were performed as described in Example 15. CFTR was stimulated by cAMP agonist forskolin and increasing concentration of test compounds was added. The compounds (Compound 6, Compound 47, Compound 17, and Compound 18) exhibited dose dependant inhibition of CFTR in short-circuit current analysis (Using chamber experiments). Tetrazolo-172 (Compound 6) and Oxo-172 (Compound 47) exhibited an IC₅₀ 0.5-1 and 1-2 μM, respectively. Inhibition by Tetrazolo-172 was slower (FIG. 2A, middle-left) than CFTRᵢₙₕ-172 (Compound 5). In control experiments, CFTRinh-172 inhibited CFTR with IC₅₀ 0.25-0.5 μM. Pyridin-NO-172 (Compound 17) and α-Methyl-172 (Compound 18) were less active, exhibiting IC₅₀ values of 7-9 and 8-10 μM, respectively.

### Example 18

### Preparation of Compound 6 (Tetrazolo-172) (T08) for Biological Analyses

Larger quantities of Compound 6 (also called herein tetrazolo-172 and T08) were prepared to perform several biology studies. For synthesis of tetrazolo-CFTRᵢₙₕ-172 (compound T08, 3-[(3-trifluoromethyl)phenyl]-5-[(4-(1H-tetrazol-5-yl)phenyl)methylene]-2-thioxo-4-thiazolidinone), a mixture of 2-thioxo-3-(3-trifluoromethylphenyl)-4-thiazolidinone (16) (100 mg, 0.36 mmol) and 4-(1H-1,2,3,4-tetrazol-5-yl)benzaldehyde (63 mg, 0.36 mmol) in absolute alcohol (1 mL) containing piperidine (1 drop) was refluxed for 30 min. The yellow precipitate was filtered, washed with ethanol, dried, and recrystallized from ethanol to give 97 mg (62% yield) of a yellow powder. Melting point 216-219° C.; ms (ES⁻): M/Z 432 (M⁺); ¹H NMR (400 MHz, DMSO-d6): 7.78 (d, 2H, carboxyphenyl, J=8.2 Hz), 7.80-8.00 (m, 5H, trifluoromethyl-phenyl and CH), 8.07 (d, 2H, carboxyphenyl, J=8.31 Hz), 13.20 (s, 1H, tetrazolo, D₂O exchange).

### Example 19

### Preparation of Compound G07 for Biological Analyses

For synthesis of Ph-GlyH-101 (compound G07, N-2-naphthalenyl-2-hydroxyethyl-[(3,5-dibromo-2,4-dihydroxyphenyl)methylene]phenylglycinehydrazide), a mixture of 2-naphthylamine (0.72 g, 5 mmol), methyl α-bromophenylacetate (1.15 g, 5 mmol) and sodium acetate (0.82 g, 10 mmol) in 1 ml of water was stirred at 80° C. for 5 h. The resultant solid after cooling was filtered and recrystallized from ethanol to yield 0.83 g ethyl N-(2-naphthalenyl) glycinate (yield 57%, melting point 137-138° C.). A solution of above product (1.45 g, 5 mmol) in ethanol (10 ml) was refluxed with hydrazine hydrate (1 g, 20 mmol) for 6 h. Solvent and excess reagents were distilled under vacuum. The product was recrystallized from ethanol to yield 1.14 g of N-(2-naphthalenyl)-α-phenyl glycine hydrazide (78%, mp 176-178° C.). A mixture of the hydrazide (2.9 g, 10 mmol) and 3,5-dibromo-4-hydroxy-benzaldehyde (2.8 g, 10 mmol) in ethanol (10 ml) was refluxed for 6 h. The hydrazide that crystallized upon cooling was filtered, washed with ethanol, and recrystallized from ethanol to give 3.64 g (yield 66%) of Ph-GlyH-101. Melting point >280° C. (decomposition), ms (ES⁻): M/Z 554 (M⁺); ¹H NMR (DMSO-d₆): δ 4.1 (s, 2H, CH), 6.5-7.5 (m, 14H, aromatic, NH), 8.5 (s, 1H, CH═N), 10.4 (s, 1H, NH—CO), 11.9 (s, 1H, OH), 12.7 (s, 1H, OH).

### Example 20

### Preparation of Compounds for Biological Analyses

Compounds T01-T07, T09-T16, G01-06 and G08-G16 were synthesized according to methods practiced in the art (see, e.g., Ma et al., J. Clin. Invest. 110:1651-1658, (2002); Muanprasat et al., J Gen Physiol 124:125-137 (2004); Sonawane et al., FASEB J 20:130-132 (2006); U.S. Pat. No. 7,235,573; U.S. Pat. No. 5,326,770; U.S. Pat. No. 6,380,186; U.S. Pat. No. 7,414,037; U.S. Patent Application Publication No. 2005/0239740; Yang et al., J. Am. Soc. Nephrol. 19:1300-10 (2008)) with minor variations.

Compound T01 is also called herein Compound 21.

Compound T02 is also called herein Compound 20.

Compound T03 is also called herein Compound 27.

Compound T04 is also called herein Compound 9.

Compound T05 is also called herein Compound 29.

Compound T06 is also called herein Compound 25.

Compound T07 is also called herein Compound 26.

Compound T08 is also called herein Compound 6 (Tetrazolo-172).

Compound T10 is also called herein Compound 14.

Compound T12 is also called herein Compound 15.

Compound T13 is also called herein Compound 38.

Compound T16 is also called herein Compound 33.

### Example 21

### CFTR Inhibitory Activity on Cyst Formation in MDCK Cell Cyst Model

a. Model of Cyst Growth

An MDCK cell model of polycystic kidney disease was used to screen CFTR inhibitors of the thiazolidinone and glycine hydrazide classes for reducing cyst formation and expansion. MDCK cells, which endogenously express CFTR (Mohamed et al., Biochem J 322: 259-265, 1997), undergo proliferation, fluid transport and matrix remodeling, as seen in tubular epithelial cells cultured from PKD kidneys, and thus provide a useful in vitro model of cystogenesis. Culture of MDCK cells in three-dimensional collagen gels produces a polarized, single-layer, thinned epithelium surrounding a fluid-filled space, apical external-facing microvilli, a solitary cilium, and apical tight junctions (see, e.g., McAteer et al., Scan Elect Microsc (Pt 3): 1135-1150, 1986; McAteer et al., Scanning Microsc 2: 1739-1763, 1988; and Taide et al., Eur J Clin Invest 26: 506-513, 1996).

Type I MDCK cells (ATCC No. CCL-34) were cultured at 37° C. in a humidified 95% air/5% CO₂ atmosphere in a 1:1 mixture of Dulbecco's modified Eagle's medium (DMEM) and Ham's F-12 nutrient medium supplemented with 10% fetal bovine serum (Hyclone), 100 U/mL penicillin and 100 μg/mL streptomycin. To generate cysts, four hundred MDCK cells were suspended in 0.4 ml of ice-cold Minimum Essential Medium containing 2.9 mg/mL collagen (PuRECOL, Inamed Biomaterials, Fremont Calif.), 10 mM HEPES, 27 mM NaHCO₃, 100 U/mL penicillin and 100 μg/mL streptomycin (pH 7.4). The cell suspension was plated onto 24-well plates. After gelation, 1.5 mL of MDCK cell medium containing 10 μM forskolin was added to each well and plates were maintained at 37° C. in a 5% CO₂ humidified atmosphere.

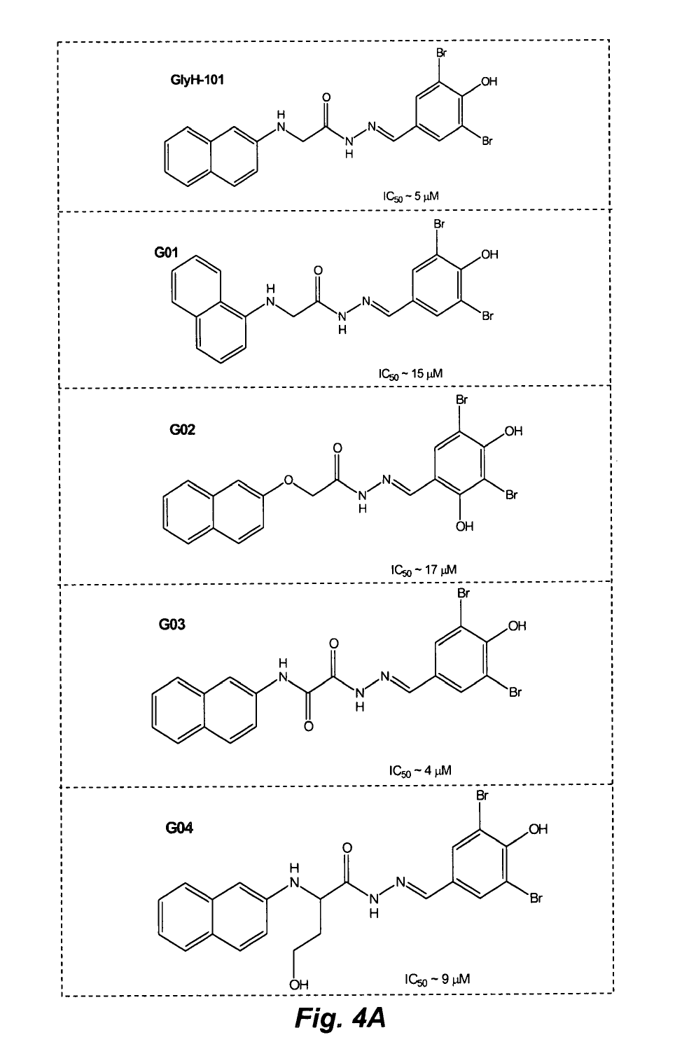

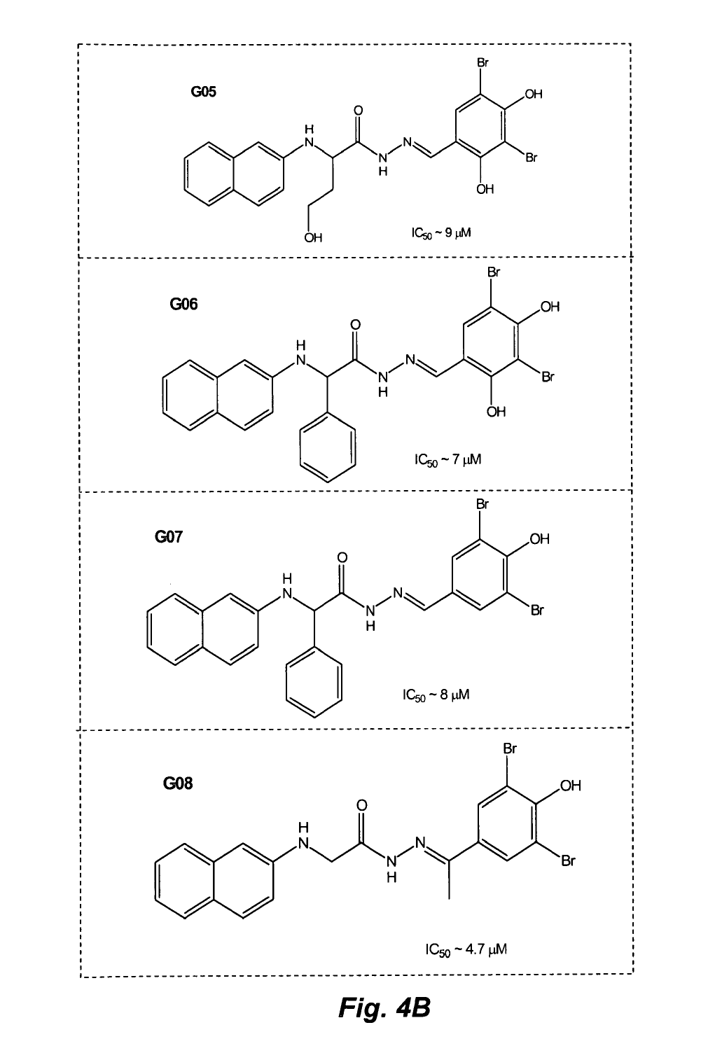

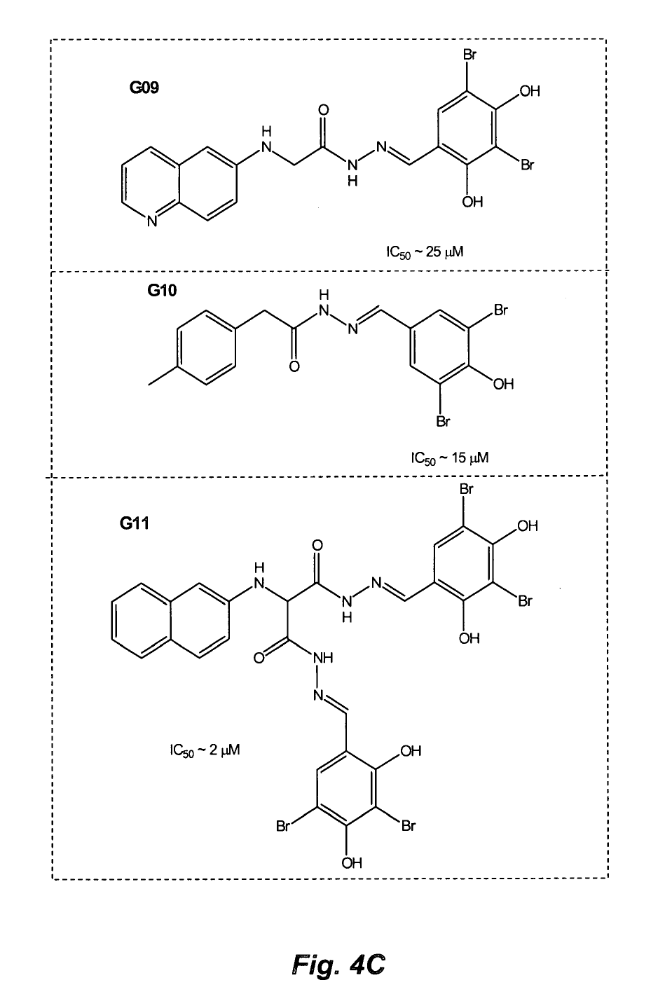

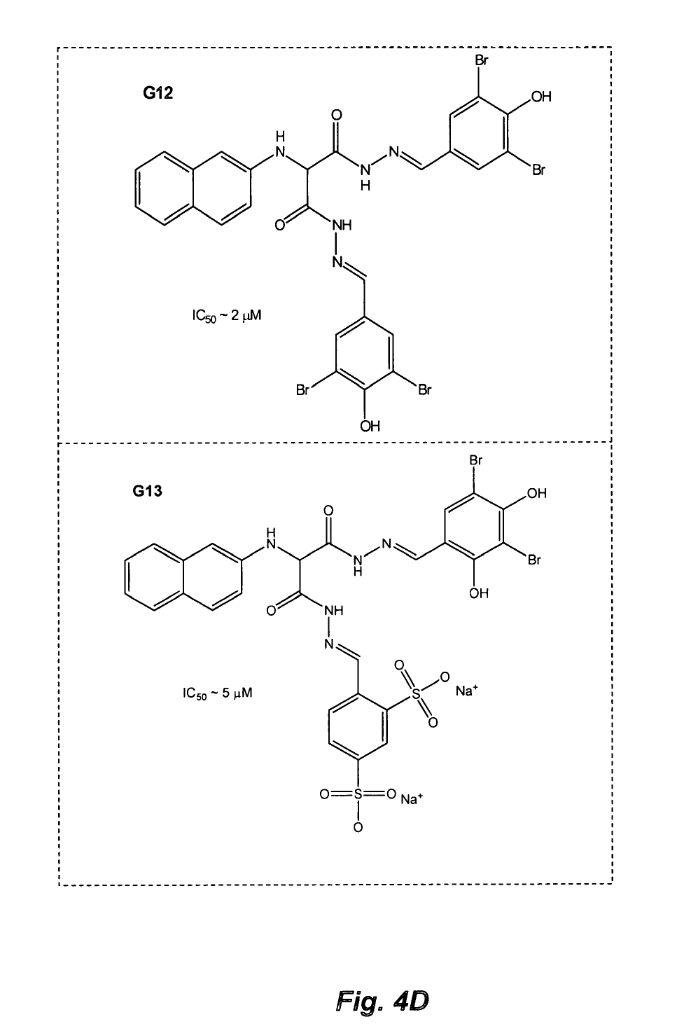

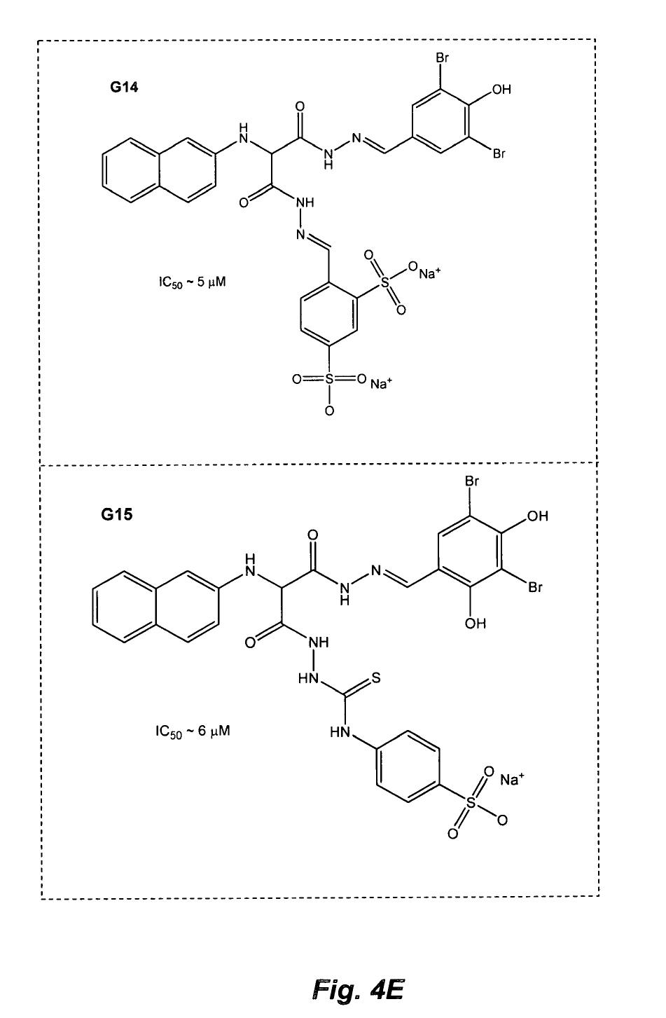

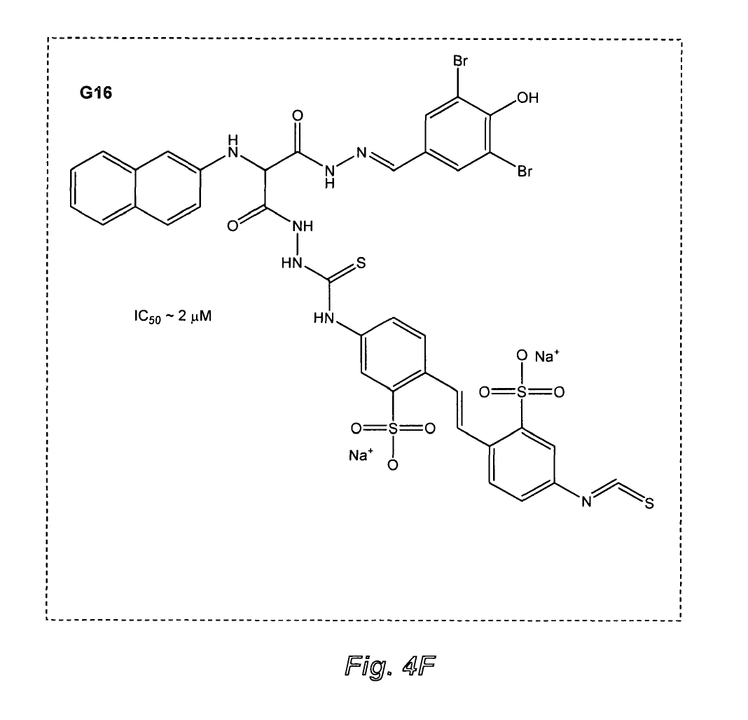

CFTR inhibitors (at 10 μM) were included in the culture medium in the continued presence of forskolin from day 0 onward. The compound structures together with their approximate CFTR inhibition potencies (expressed as IC₅₀ values) are provided in FIGS. 3 and 4. Indicated IC₅₀ values for T01-07, T10, T12-14 were reported by Ma et al. (J Clin Invest 110: 1651-1658 (2002)). IC₅₀ values for T08, T11, and T16 were determined by short-circuit analysis performed according to methods described herein. Indicated IC₅₀ values for G01-05, G8-16 were reported by Sonawane et al. (FASEB J. 20: 130-132 (2006); Sonawane et al., Gastroenterology 132:1234-44 (2007)). IC₅₀ values for G06 and G07 were determined by short-circuit current analysis performed according to methods described herein.

Medium containing forskolin and test compounds was changed every 12 h. At day 6, cysts (with diameters >50 μm) and non-cyst cell colonies were counted by phase-contrast light microscopy at 20× magnification (546 nm monochromatic illumination) using a Nikon TE 2000-S inverted microscope. In some experiments, compounds were added to medium in the continued presence of forskolin from day 4 after seeding and the medium containing forskolin and compounds was changed every 12 h for 8 days. Micrographs showing the same cysts in collagen gels (identified by markings on plates) were obtained every 2 days. For determination of cyst growth, cyst diameters were measured using Image J software. At least 10 cysts/well and 3 wells/group were measured for each condition.

Cysts were seen in 3 to 4 days, progressively enlarging over the next 8 days (FIG. 2A, top), and did not form in the absence of forskolin. FIG. 2E shows that the total numbers of colonies (cysts plus non-cyst colonies) were similar in the control and inhibitor-treated groups.

Exposure of established cysts (>50 μm diameter on day 4) to a CFTR inhibitor (compound T08) at 10 μM for 8 days slowed cyst enlargement (FIG. 2A, middle) Inhibition was reversible as shown by exposure to inhibitor at days 4-8 followed by washout (FIG. 2A, bottom). Eight compounds, including T08, T14, G07 and G16, inhibited cyst growth by >70% at 10 μM (FIG. 2B). CFTRᵢₙₕ-172 also inhibited cyst formation, but to a lesser extent (FIG. 2B). Upon further testing as described above, compounds G07 (a glycine hydrazide analog) and T08 (a thiazolidionone analog) strongly inhibited cyst enlargement at 1 μM (FIG. 2D).

b. Cytotoxicity, Cell Proliferation, and Apoptosis

To test whether inhibition of cyst growth could be related to cytotoxicity, certain compounds were tested for their effects on cell viability, cell proliferation and apoptosis. Crystal violet staining was used to assess compound effects on cytotoxicity (see, e.g., Johnson et al., Clin Cancer Res 11: 6924-6932, 2005). MDCK cells were incubated for 24 h on 96-well plates, and then incubated for 72 h with test compounds at 20 μM. Medium was removed and adherent cells were fixed and stained for 30 min with 0.5% crystal violet in 20% methanol. Plates were washed with distilled water, and the stain was extracted with Sorenson's buffer (0.1 mol/L sodium citrate, pH 4.2, in 50% ethanol) overnight at 4° C., and absorbance measured at 570 nm.

Cell proliferation was assayed using a BrdU cell proliferation assay kit (CALBIOCHEM, San Diego, Calif.). MDCK cells (10⁴/well) were seeded on 96-well plates and incubated for 72 h with test compounds at 5, 10 or 20 μM. BrdU was added at 60 h of culture. BrdU incorporation was measured according to manufacturer's instructions by absorbance at 490 nm.

Apoptosis was measured using the in situ cell death detection kit (ROCHE Diagnostics, Indianapolis, Ind.). MDCK cells were seeded on 8-chamber polystyrene tissue culture-treated glass slides and incubated with compounds T08 and G07 for 72 h at 5, 10 or 20 μM. The assay was performed according to manufacturer's instructions. Five microscopic fields were analyzed per condition. The apoptosis index was calculated as the percentage of nucleus-stained cells.

At 20 μM, compounds T09, T12, T13, G04, and G05 reduced MDCK cell viability, whereas compounds CFTRᵢₙₕ-172, T08, T14, G03, G07 and G16 did not (FIG. 2C). At 10 μM, compounds T08 and G07 did not cause MDCK cell apoptosis (FIG. 2H). The MDCK cyst model identified CFTR inhibitors that reduced cyst formation and enlargement without demonstrable cell toxicity and without inhibiting cell proliferation. Compounds T08 and G07, representing potent and non-toxic compounds of the glycine hydrazide and thiazolidinone classes, respectively, were further evaluated.

c. Short-Circuit Current Measurements

CFTR inhibition potency was confirmed in MDCK cells by short-circuit current analysis. Snapwell inserts containing MDCK cells (transepithelial resistance 1000-2000 Ohms) were mounted in a standard Using chamber system. The basolateral membrane was permeabilized with 250 μg/ml amphotericin B. The hemichambers were filled with 5 mL of 65 mM NaCl, 65 mM Na-gluconate, 2.7 mM KCl, 1.5 mM KH₂PO₄, 1 mM CaCl₂, 0.5 mM MgCl₂, Na-Hepes and 10 mM glucose (apical), and 130 mM NaCl, 2.7 mM KCl, 1.5 mM KH₂PO₄, 1 mM CaCl₂, 0.5 mM MgCl₂, Na-Hepes and 10 mM glucose (basolateral) (pH 7.3). Short-circuit current was recorded continuously using a DVC-1000 voltage clamp (World Precision Instruments, Sarasota Fla.) with Ag/AgCl electrodes and 1 M KCl agar bridges.

In some experiments, MDCK cells in Snapwell inserts were cultured in medium containing 10 μM T08 or G07 for 1 or 48 hours. Compounds were washed out with medium for 1 hour before short-circuit current measurements. FIG. 2F shows the concentration-dependent inhibition of short-circuit current following CFTR simulation by forskolin, with IC₅₀s of ˜1 μM. FIG. 2H shows that T08 and G07 (at 10 μM) did not alter CFTR expression, as seen by similar short-circuit current measurements in MDCK cells after 1 or 48 hour incubation with the compounds.

### Example 22

### CFTR Inhibitory Activity on Cyst Development and Growth in Embryonic Kidney Culture

An embryonic kidney organ culture model was used to further evaluate compounds T08 and G07. Embryonic kidney culture models permit organotypic growth and differentiation of renal tissue in defined media without the confounding effects of circulating hormones and glomerular filtration (see, e.g., Magenheimer et al., J Am Soc Nephrol 17: 3424-37, 2006; and Gupta et al., Kidney Int 63: 365-376, 2003). In addition, the early mouse kidney tubule in this model has an intrinsic capacity to secrete fluid in response to cAMP by a CFTR-dependent mechanism (Magenheimer et al.), allowing the evaluation of CFTR inhibitors in particular.

Mouse embryos were obtained at embryonic day 13.5 (E13.5). Metanephroi were dissected and placed on transparent Falcon 0.4-mm diameter porous cell culture inserts. To the culture inserts was added DMEM/Ham's F-12 nutrient medium supplemented with 2 mM L-glutamine, 10 mM HEPES, 5 μg/ml insulin, 5 μg/ml transferrin, 2.8 nM selenium, 25 ng/ml prostaglandin E, 32 pg/ml T3, 250 U/ml penicillin and 250 μg/ml streptomycin. Kidneys were maintained in a 37° C. humidified CO₂ incubator, and were cultured for 4 days in the absence or presence of 100 μM 8-Br-cAMP. Culture medium containing 100 μM 8-Br-cAMP, with or without CFTR inhibitors, was replaced (in the lower chamber) every 12 hours.

Kidneys were photographed using a Nikon inverted microscope (Nikon TE 2000-S) equipped with 2× objective lens, 520 nm bandpass filter, and high-resolution PixeLINK color CCD camera. Cyst area was calculated as total cyst area divided by total kidney area. Cyst sizes in micrographs of metanephroi were determined using MATLAB 7.0 software. A masking procedure was used to highlight all pixels of similar intensity within each cyst. Fractional cyst area was calculated as total cyst area divided by total kidney area. Cysts with diameters >50 μm were included in the analysis. Image acquisition and analysis was done without knowledge of treatment condition.

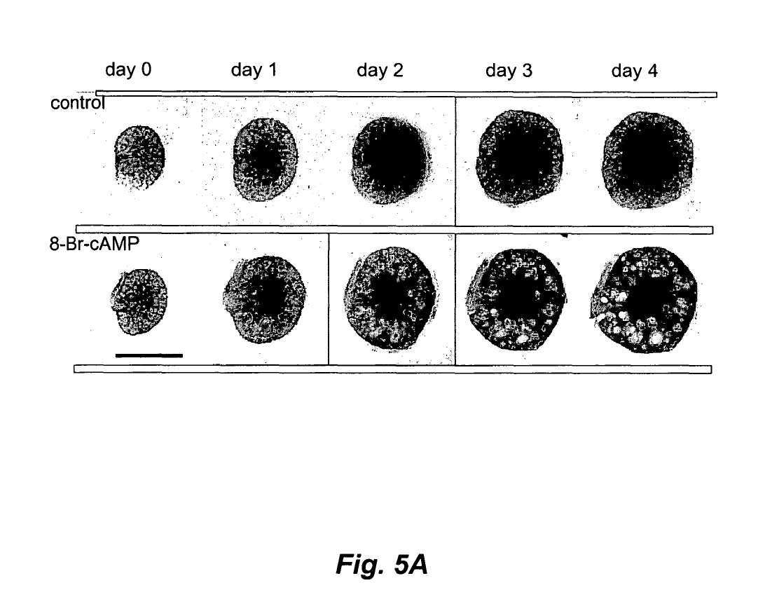

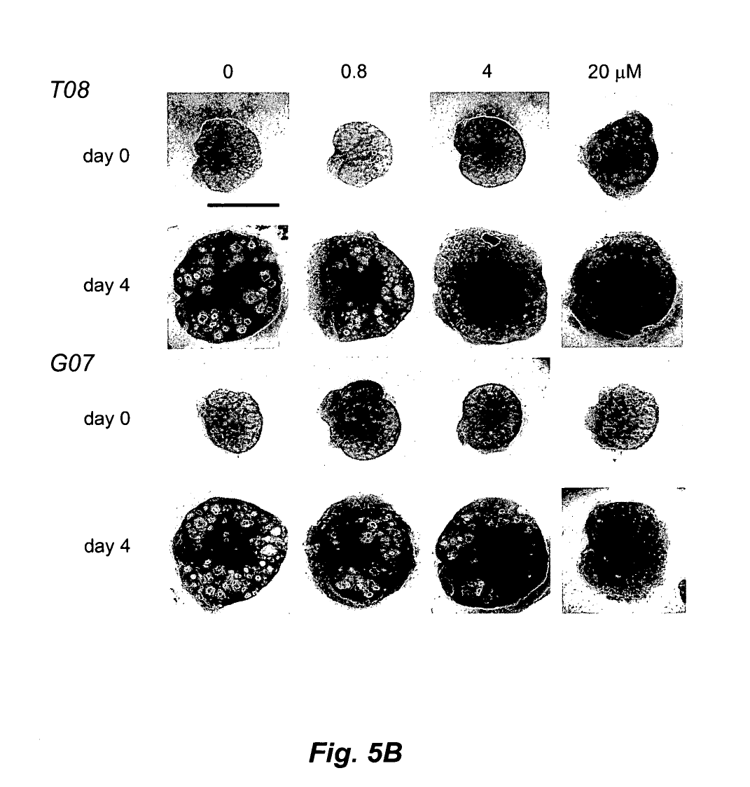

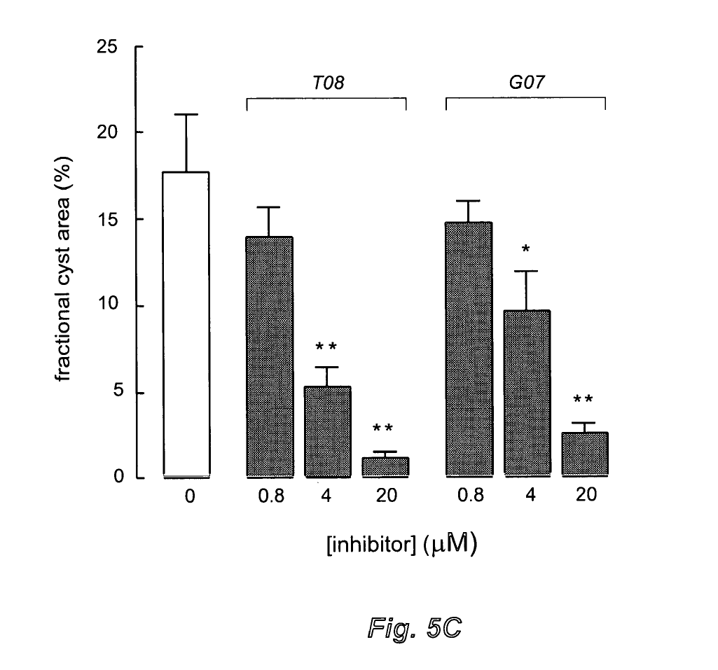

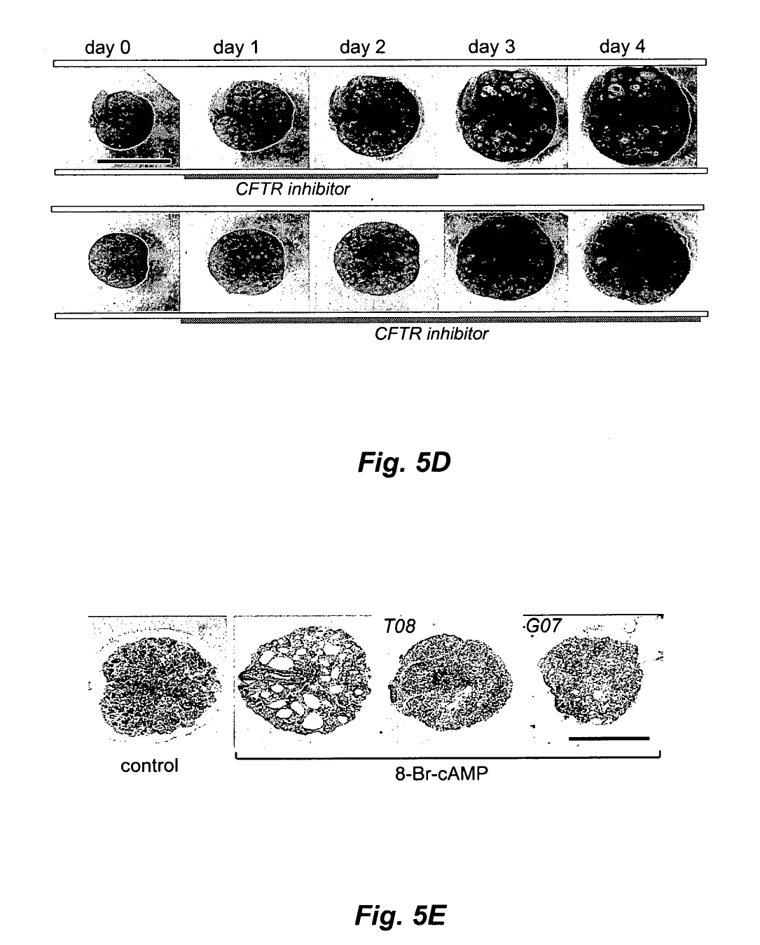

In the absence of 8-Br-cAMP, kidneys increased in size over 4 days (FIG. 5A, top panel), whereas numerous cystic structures were seen in the presence of 8-Br-cAMP (FIG. 5A, bottom panel). FIG. 5B shows that compounds T08 and G07 remarkably reduced cyst formation, as confirmed by quantitative image analysis (FIG. 5C). In control studies, cysts formed following compound washout after two-day treatment (FIG. 5D), indicating reversible action of the T08 and G07 CFTR inhibitors. Also, kidney growth in the absence of 8-Br-cAMP was not affected by the CFTR inhibitors: after 4 days in culture kidney lengths were 7.4±0.5 mm (T08-treated), 7.1±0.4 mm (G07-treated) and 7.3±0.4 mm (control).

Paraffin sections are shown in FIG. 5E. In the absence of 8-Br-cAMP, renal tubules and primitive distal ramifications of the ureteric bud formed after 4 days in culture. Large cystic structures were seen throughout the kidney in the presence of 8-Br-cAMP. Compounds T08 and G07 reduced the number and size of cysts (FIG. 5E). The apoptotic index was <1% in kidneys exposed to T08 or G07 at 20 μM. These data show that the CFTR inhibitors T08 and G07 reversibly inhibited cyst formation and growth in embryonic kidneys without measurable effects on kidney growth, and without measurable cell toxicity.

### Example 23

### CFTR Inhibitory Activity on Cyst Development in a Mouse Model of Autosomal Dominant Polycystic Kidney Disease

An in vivo model of kidney disease was employed to further explore the ability of CFTR inhibitors to reduce cyst formation in a vascular, perfused cellular environment. Pkd1^{flox/-}; Ksp-Cre mice were employed for this model, since these kidney-selective Pkd1 knockout mice manifest a fulminant course, with development of large cysts and renal failure in first 2 weeks of life, and represent a postnatal model of ADPKD. Pkd1 knockout mice normally die within 20 days. The Pkd1^{flox/-}; Ksp-Cre model is suitable, inter alia, for evaluating the efficacy of CFTR inhibitors on retarding the growth of cysts in the distal segments of the nephron, including medullary thick ascending limbs of the loops of Henle, distal convoluted tubule and collecting ducts.

Pkd1^{flox} mice and Ksp-Cre transgenic mice in a C57BL/6 background were generated as described (see, e.g., Shibazaki et al., J Am Soc Nephrol 13: 10-11, 2004; and Shao et al., J Am Soc Nephrol 13: 1837-1846, 2002). Ksp-Cre mice express Cre recombinase under the control of the Ksp-cadherin promoter (Shao et al.). Kidney-specific Pdk1 knock-out mice (Pkd1^{flox/-}; Ksp-Cre mice) were generated by cross-breeding Pkd1^{flox/flox} mice with Pkd1^{+/-}:Ksp-Cre mice. Neonatal mice (age 1 day) were genotyped by genomic PCR.

CFTR inhibitors (5-10 mg/kg/day) or saline DMSO vehicle control (0.05 mL/injection) were administrated by subcutaneous injection on the backs of neonatal mice four times a day for 3 or 7 days using a 1 cc insulin syringe, beginning at age 2 days (11 mice per group). Pkd1^{flox/+}; Ksp-Cre or Pkd1^{flox/+} mice from the same litter were used as controls. During the treatment period, control and Pkd1^{flox/-}; Ksp-Cre mice, with or without CFTR inhibitor treatment, were indistinguishable in their activity and behavior. Body weight was measured at day 5 (3 days after treatment), at which time there was no difference in body weight in any of the mouse groups. Blood and urine samples were collected for measurements of CFTR inhibitor concentration and renal function. Kidneys were removed and weighed, and fixed for histological examination or homogenized for determination of CFTR inhibitor content.

a. Measurements of CFTR Concentration

For high-performance liquid chromatography/mass spectrometry (HPLC/MS) analysis of CFTR inhibitor concentration, kidneys were homogenized in 50-100 μl of PBS for 5 min using an EPPENDORF pellet pestle homogenizer. The homogenate was mixed with an equal volume of chilled acetonitrile to precipitate proteins. After centrifugation at 5000×g for 10 min the supernatant was evaporated under nitrogen, and the residue was dissolved in eluent (50% CH₃CN/20 mM NH₄OAc). Urine samples were directly diluted 10-fold with eluent. Reversed-phase HPLC separations were carried out using a WATERS C18 column (2.1×100 mm, 2.5 μm particle size) equipped with a solvent delivery system (WATERS model 2690, Milford, Mass.). The solvent system consisted of a linear gradient from 20% CH₃CN/20 mM NH₄OAc to 95% CH₃CN/20 mM NH₄OAc, run over 20 min, followed by 5 min at 95% CH₃CN/20 mM NH₄OAc (0.2 mL/min flow rate). Mass spectra were acquired on an Alliance HT 2790+ZQ mass spectrometer using negative ion detection, scanning from 150 to 1500 Da. The electrospray ion source parameters were: capillary voltage 3.2 kV (negative ion mode) or 3.5 kV (positive ion mode), cone voltage 37 V, source temperature 120° C., desolvation temperature 250° C., cone gas flow 25 L/h, and dessolvation gas flow 350 L/h.

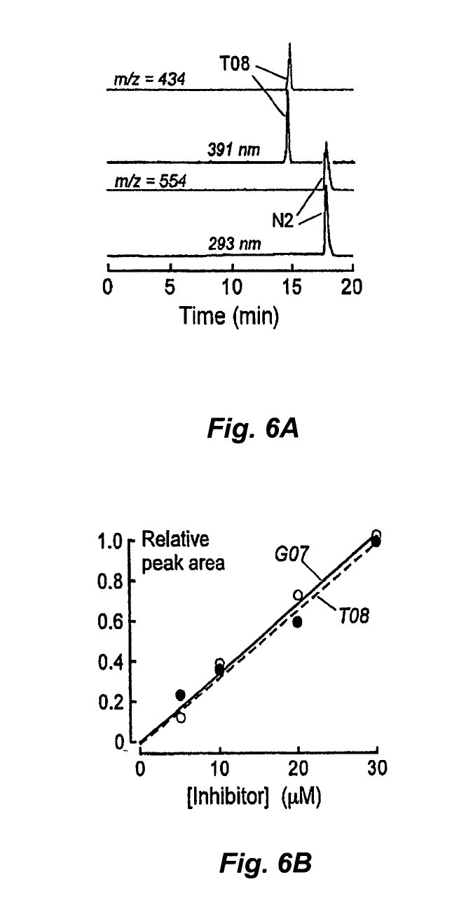

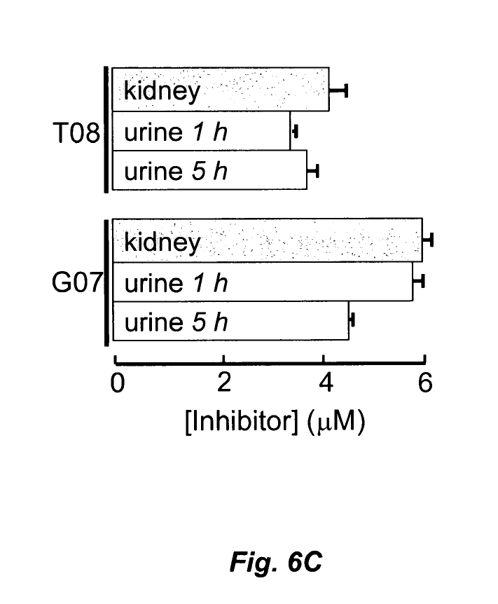

Representative HPLC and mass chromatograms are provided in FIG. 6A, showing 50 picomolar sensitivity. Tetrazolo-CFTRᵢₙₕ-172 (compound T08) and Ph-GlyH-101 (compound G07) were detected by absorbance at 386 nm and 338 nm, respectively, with mass traces of m/z 433.4 Da and 553.2 Da. Assays were linear over 0.05-15 μg/ml, with 0.01 μg/ml detection limit. Assay sensitivity and specificity were confirmed by adding known quantities of inhibitors to urine from non-compound-treated mice (FIG. 6B).

Concentrations were measured to establish dosing to give sustained concentrations in kidney/urine of >1 μM, where CFTR is inhibited. Kidney and urine samples were obtained from mice after 4 times daily subcutaneous administration for 3 days at 5 mg/kg/day, a dose regimen determined from preliminary studies. Urinary concentrations were measured at 1 and 5 hour after the final dosing. For tetrazolo-CFTRᵢₙₕ-172, urine concentrations were 3.3 and 3.6 μM at 1 and 5 hour, respectively. The urine concentrations of Ph-GlyH-101 were 4.3 and 5.8 μM. Comparable inhibitor concentrations were found in kidney homogenates. These concentrations are several-fold greater than the IC₅₀ for CFTR inhibition.

These data indicate that effective CFTR inhibitory concentrations of >3 μM in urine and kidney tissue were obtained by subcutaneous compound administration at 5-10 mg/kg/day every 6 hours from days 2-5.

b. Measurements of Renal Function

To measure serum creatinine and urea (indicators of renal function), serum was obtained from whole blood by centrifugation at 5000×g for 5 min. Serum creatinine concentration was measured using a colorimetric assay kit (Cayman Chemical, Ann Arbor Mich.) following manufacturer's instructions. Urea concentration was measured using the colorimetric QUANTICHROM Urea Assay Kit (BioAssay Systems, Hayward, Calif.). Creatinine and urea concentrations were determined from optical densities using calibration standards.

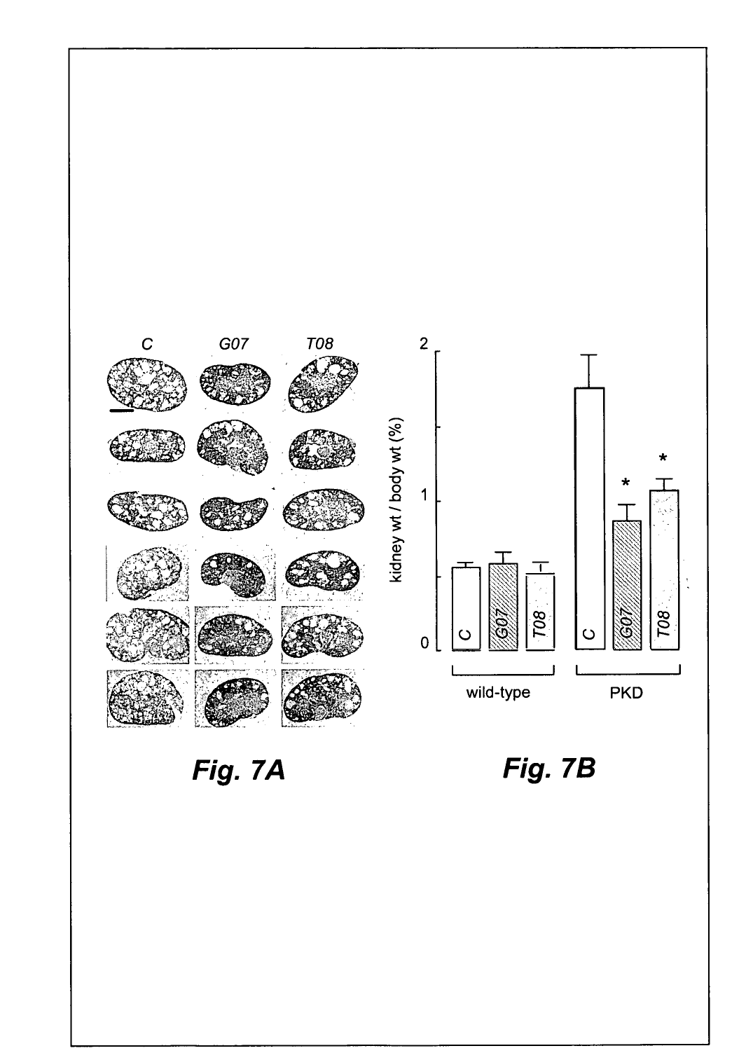

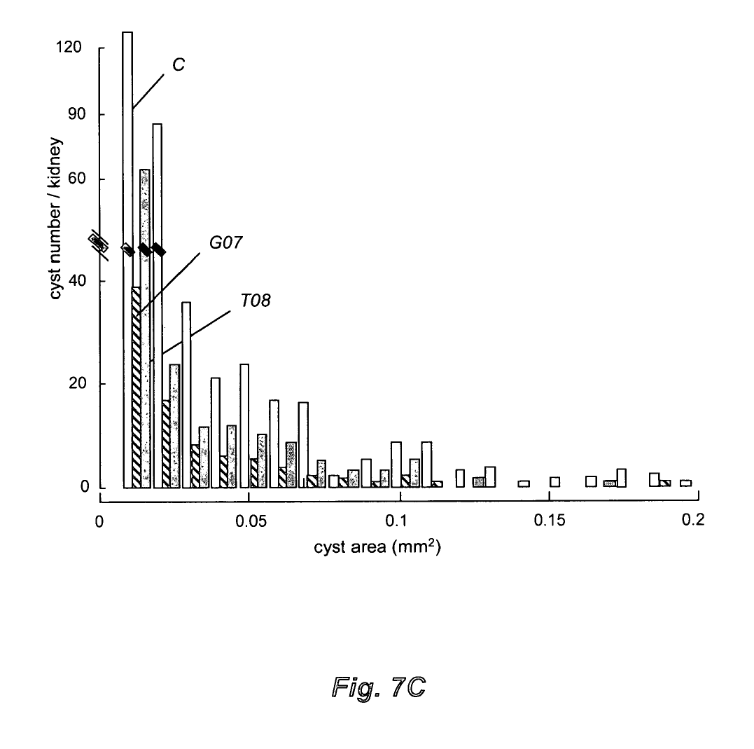

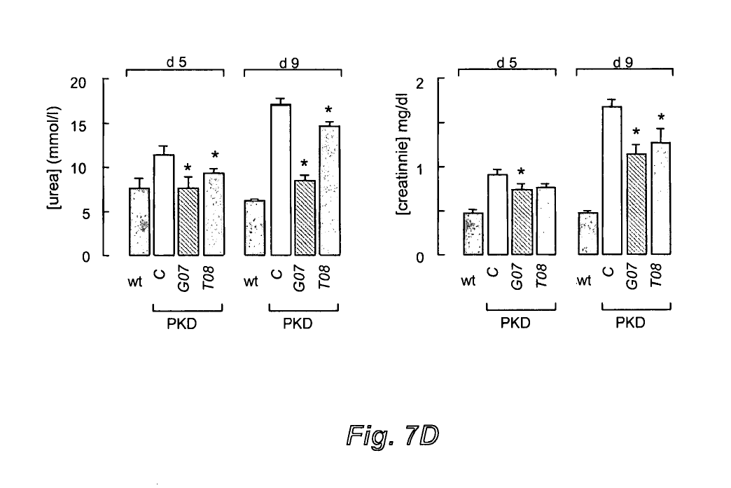

FIG. 7D shows mild elevations in serum creatinine and urea in vehicle-treated Pkd1^{flox/-}; Ksp-Cre mice (C), as compared to wild-type mice (wt), at day 5 (d5), with more marked elevation at day 9 (d9). Serum creatinine and urea were significantly reduced in T08 and G07-treated Pkd1^{flox/-}; Ksp-Cre mice, demonstrating improved renal function as compared to untreated control Pkd1^{flox/-}; Ksp-Cre mice (C).

c. Histological Examination

For histological examination, kidneys were fixed with Bouin's fixative and embedded in paraffin. Three-μm thick sections were cut serially every 200 μm and stained with hematoxylin and eosin (H&E). Sections were imaged using a LEICA inverted epifluorescence microscope (DM 4000B) equipped with 2.5× objective lens and color CCD camera (Spot, model RT KE; Diagnostic Instruments Inc.). Cyst sizes in micrographs of kidney were determined using MATLAB 7.0 software.

FIG. 7A shows central coronal kidney sections. Although there was some mouse-to-mouse variability, kidney sections from T08 and G07-treated mice showed fewer cysts of all sizes. Kidney weights in T08- and G07-treated wild-type mice were similar to those in untreated control mice (FIG. 7B). Kidney weight in Pkd1^{flox/-}; Ksp-Cre mice was >3-fold higher than in wild-type mice. Treatment of Pkd1^{flox/-}; Ksp-Cre mice with compounds T08 or G07 reduced kidney weight significantly compared with vehicle-treated Pkd1^{flox/-}; Ksp-Cre mice. Image analysis of H&E sections showed remarkably fewer total numbers of cysts (of >50 μm diameter) per kidney in T08- and G07-treated mice (797±69, control; 457±32, T08; 316±45, G07), with reduced numbers of medium- and large-size cysts (FIG. 7C).

The T08 and G07 CFTR inhibitors described herein significantly reduced both cyst formation and clinical signs of PKD, as assessed by lower kidney weights, and serum creatinine and urea concentrations. These data not only show that thiazolidinone- and glycine hydrazide-type small-molecule CFTR inhibitors, at concentrations without apparent toxicity or inhibition of cell proliferation, retarded the growth of renal cysts in in vitro and in vivo PKD models, but support the conclusion that CFTR-dependent fluid secretion is an important determinant in the development and growth of renal epithelial cell cysts.

All U.S. patents, U.S. patent application publications, U.S. patent applications, foreign patents, foreign patent applications, and non-patent publications referred to in this specification and/or listed in the Application Data Sheet, are incorporated herein by reference, in their entirety.

From the foregoing, although specific embodiments have been described herein for purposes of illustration, various modifications may be made without deviating from the spirit and scope of the invention. Those skilled in the art will recognize, or be able to ascertain, using no more than routine experimentation, many equivalents to the specific embodiments described herein. Such equivalents are intended to be encompassed by the following claims.

1. A compound having the following structure I: 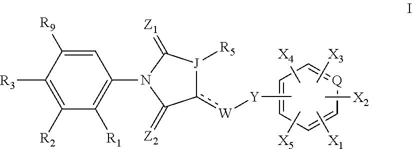
or a pharmaceutically acceptable salt, prodrug, or stereoisomer thereof, wherein
Y is —NH— or absent;
W is ═CH—, —S—, —O—, —C(═S)—, or —C(═O)—;
Z₁, Z₂, Z₃, Z₄, and Z₅ are each independently O or S;
J is S;
Q is N;
R₁, R₂, R₃, and R₉ are each independently H, C₁₋₆ alkyl, alkoxy, halo, —CF₃, —CF₂CF₃, or —OCF₃ wherein at least one of R₁, R₂, R₃, and R₉ is —CF₃, or —CH₃;
R₅ is absent;
X₁, X₂, X₃, and X₄ are each independently H, —OH, —SH, halo, tetrazolo, —P(═O)(OH)₂, —C(═Z₃)Z₄H, —Z₅—C(═Z₃)Z₄H, or —Z₅—CH₂—C(═Z₃)Z₄H; and
X₅ is —O⁻, tetrazolo, —C(═O)OH, —O—C(═O)OH, or absent.

2. (canceled)

3. A compound having the following structure I(A): 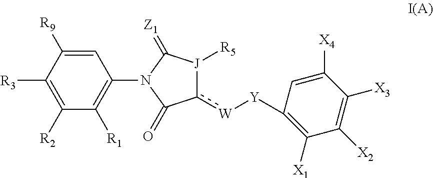
or a pharmaceutically acceptable salt, prodrug, or stereoisomer thereof, wherein
Y is —NH— or absent;
W is ═CH—, —S—, —C(═S);
Z₁, Z₃, Z₄, and Z₅ are each independently O or S;
J is C or S;
R₁, R₂, R₃, and R₉ are each independently H, C₁₋₆ alkyl, alkoxy, halo, —CF₃, —CF₂CF₃, or —OCF₃ wherein at least one of —CH₃, —CF₃, —CF₂CF₃, or —OCF₃;
R₅ is H, halo, C₁₋₆ alkyl, or absent; and
X₁, X₂, X₃, and X₄ are each independently H, —OH, —SH, halo, tetrazolo, —P(═O)(OH)₂, —C(═Z₃)Z₄H, —Z₅—C(═Z₃)Z₄H, or —Z₅—CH₂—C(═Z₃)Z₄H; and wherein
(A) at least one of X₁, X₂, X₃, and X₄ is tetrazolo or —Z₅—CH₂—C(═Z₃)Z₄H;
(B) at least one of R₁, R₂, R₃, and R₉ is —CF₃, and at least one R₁, R₂, R₃, and R₉ is halo or —CH₃;
(C) at least one of R₁, R₂, R₃, and R₉ is —CH₃, wherein
(i) each of X₁, X₂, and X₄ is H, and X₃ is —C(═O)OH, —O—C(═O)OH, or —O—CH₂—C(═O)OH;
(ii) each of X₁, X₂, and X₄ is H, and X₃ is —C(═O)OH;
(iii) X₁ and X₄ are each H, X, X₂ is —OH and X₃ is —C(═O)OH;
(iv) X₁ and X₄ are each H, X₂ is —C(═O)OH, and X₃ is —OH; or
(v) X₁ is H or —OH, X₂ and X₄ are each bromo, and X₃ is —OH;
(D) J is S and R₅ is absent; Y is absent; W is ═CH—; and
(I) at least one of R₁, R₂, R₃, and R₉ is —CF₃ and the remaining of R₁, R₂, R₃, and R₉ are each independently halo, —CF₃, —CH₃, or H; and
(a) at least one of X₁, X₂, X₃, and X₄ is —OH, and at least one of the remaining X₁, X₂, X₃, and X₄ is —C(═O)OH or —OCH₂C(═O)OH;
(b) at least one of X₁, X₂, X₃, and X₄ is —OCH₂C(═O)OH;
or (c) at least 3 of X₁, X₂, X₃, and X₄ are —OH; or
(II) at least two of R₁, R₂, R₃, and R₉ are not H; and X₁, X₂, X₃, and X₄ are each independently H, —OH, —C(═O)OH, or —OCH₂C(═O)OH; or
(E) J is S and R₅ is absent; Y is absent; W is ═CH—; and each of X₁ and X₃ is —OH and each or X₂ and X₄ is bromo.

4. (canceled)

5. The compound of claim 3, wherein J is S and R₅ is absent.

6.-17. (canceled)

18. The compound of claim 3 wherein J is S, R₅ is absent, and X₃ is tetrazolo-5-yl and the compound has the following structure I(A1): 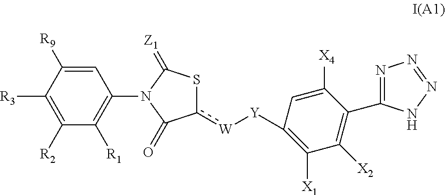
or a pharmaceutically acceptable salt, prodrug, or stereoisomer thereof, wherein
Y is —NH— or absent
W is ═CH—, —S—, or —C(═S)—;
Z₁, Z₃, Z₄, and Z₅ are each independently O or S;
R₁, R₂, R₃, and R₉ are each independently H, C₁₋₆ alkyl, alkoxy, halo, —CF₃, —CF₂CF₃, or —OCF₃ wherein at least one of R₁, R₂, R₃, and R₉ is —CH₃, —CF₃, —CF₂CF₃, or —OCF₃; and
X₁, X₂, and X₄ are each independently H, —OH, —SH, halo, —P(═O)(OH)₂, —C(═Z₃)Z₄H, —Z₅—C(═Z₃)Z₄H, or —Z₅—CH₂—C(═Z₃)Z₄H.

19. (canceled)

20. (canceled)

21. The compound claim 18, wherein X₁, X₂, and X₄ are each independently H, —OH, bromo, —C(═O)OH, or —OCH₂C(═O)OH.

22. The compound of claim 18, wherein Y is absent, and each of X₁, X₂, and X₄ is H, and the compound has the following structure I(A2), or the following structure I(A3): 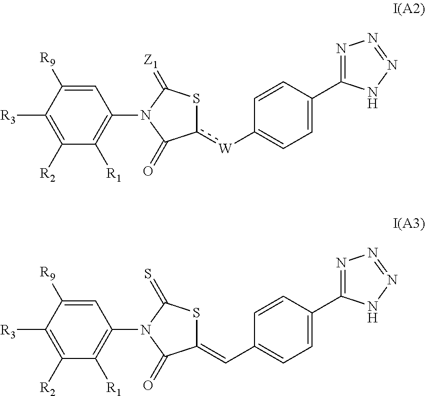
wherein
W is ═CH— or —S—;
Z₁ is O or S;
R₁, R₂, R₃, and R₉ are each independently H, —CH₃, OCH₃, chloro, fluoro, —CF₃, —CF₂CF₃, or —OCF₃ wherein at least one of R₁, R₂, R₃, and R₉ is —CH₃, or —CF₃.

23. (canceled)

24. (canceled)

25. The compound of claim 18 wherein Z₁ is O, Y is absent, W is ═CH—, and the compound has the following structure I(A4): 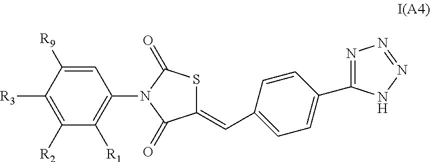
wherein R₁, R₂, R₃, and R₉ are each independently H, C₁₋₆ alkyl, —OCH₃, chloro, fluoro, —CF₃, —CF₂CF₃, or —OCF₃ wherein at least one of R₁, R₃, and R₉ is —CH₃, or —CF₃.

26. (canceled)

27. (canceled)

28. The compound of claim 25, wherein at least two of R₁, R₂, R₃, and R₉ are H.

29. (canceled)

30. The compound of claim 3, wherein the compound has the following structure selected from:

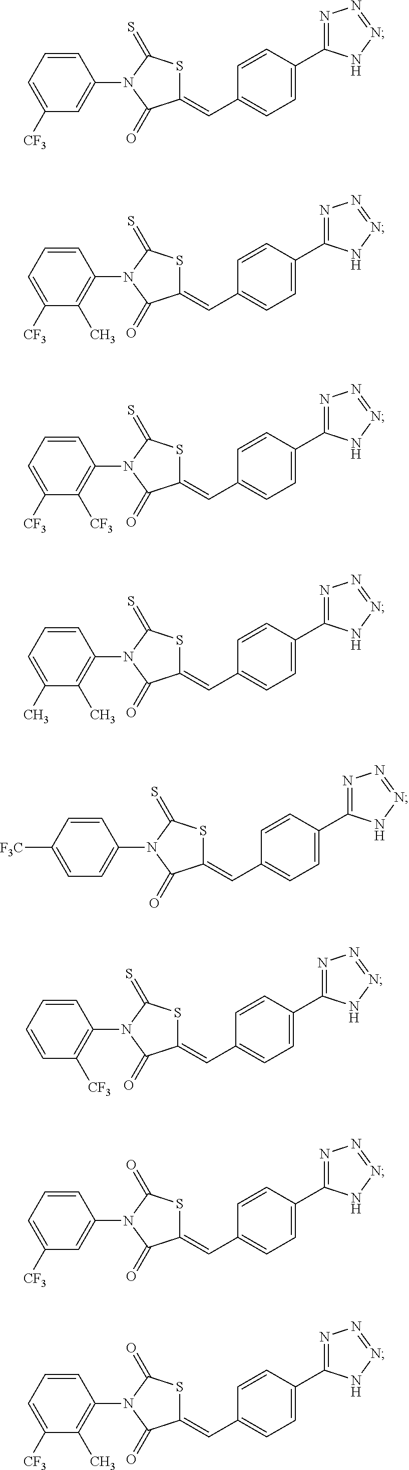

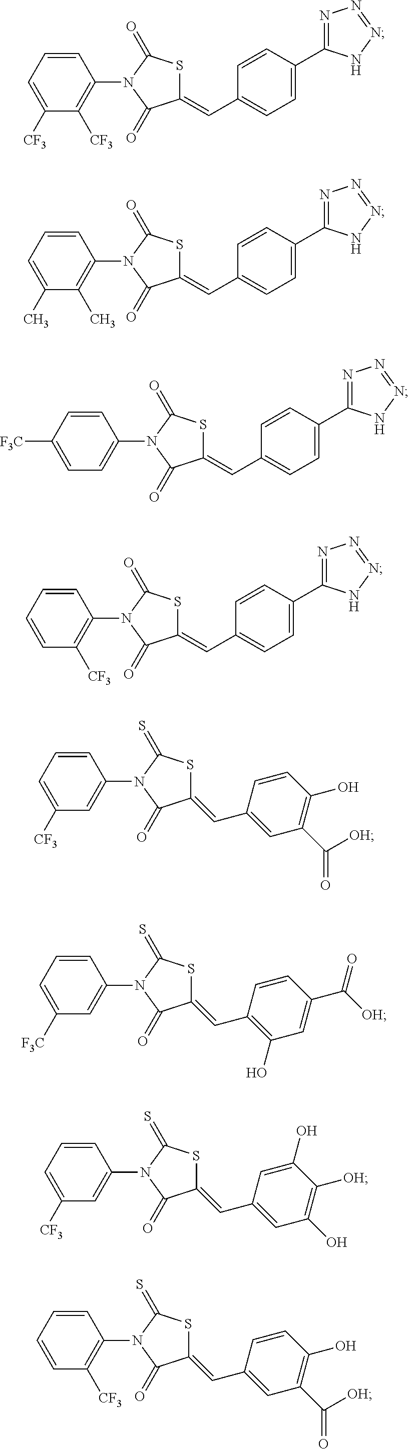

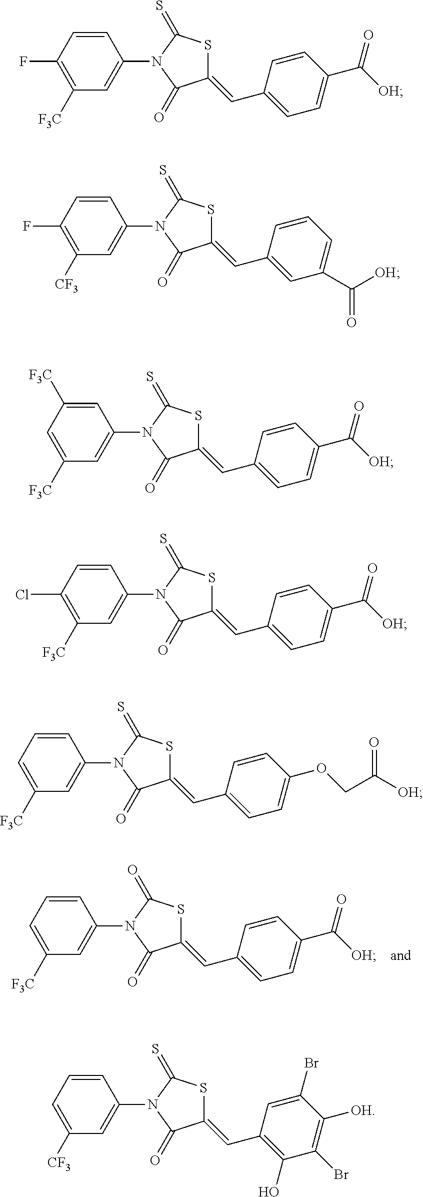

31. The compound of claim 3 wherein J is S, R₅ is absent, Y is absent, and W is ═CH— and the compound has the following structure I(A5): 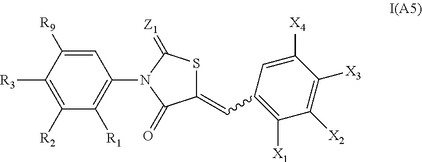
or a pharmaceutically acceptable salt, prodrug, or stereoisomer thereof, wherein
Z₁, Z₃, Z₄, and Z₅ are each independently O or S;
R₁, R₂, R₃, and R₉ are each independently H, C₁₋₆ alkyl, alkoxy, halo, —CF₃, —CF₂CF₃, or —OCF₃ wherein at least one of R₁, R₂, R₃, and R₉ is —CH₃; and
(a) each of X₁, X₂, and X₄ is H, and X₃ is —C(═O)OH, —O—C(═O)OH, or —O—CH2-C(═OH)OH;
(b) each of X₁, X₂, and X₄ is H, and X₃ is —C(═O)OH;
(c) X₁ and X₄ are each H, X₂ is —OH, and X₃ is —C(═O)OH;
(d) X₁ and X₄ are each H, X₂ is —C(═O)OH, and X₃ is —OH; or
(e) X₁ is H or —OH, X₂ and X₄ are each bromo, and X₃ is —OH.

32.-35. (canceled)

36. The compound of claim 31, wherein at least one of the remainder of R₁, R₂, R₃, and R₉ is —CF₃.

37. (canceled)

38. The compound of claim 31 wherein the compound has a structure selected from:

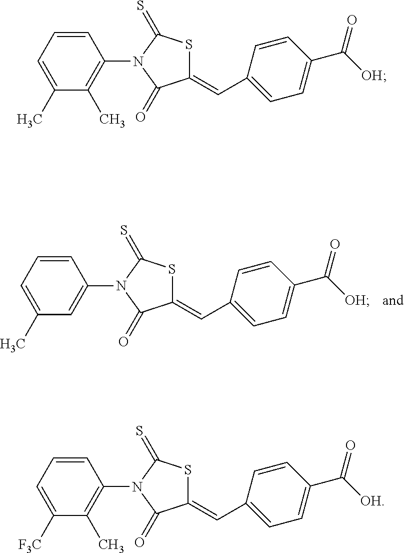

39. A compound having the following structure I(A6) or I(A7): 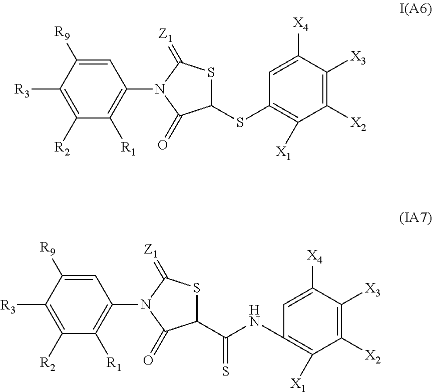
or a pharmaceutically acceptable salt, prodrug, or stereoisomer thereof, wherein
Z₁, Z₃, Z₄, and Z₅ are each independently O or S;
R₁, R₂, R₃, and R₉ are each independently H, C₁₋₆ alkyl, alkoxy, chloro, fluoro, —CF₃, —CF₂CF₃, or —OCF₃, wherein at least one of R₁, R₂, R₃, and R₉ is —CF₃ or —CH₃; and
X₁, X₂, X₃, and X₄ are each independently H, —OH, —SH, halo, —P(═O)(OH)₂, —C(═Z₃)Z₄H, —Z₅—C(═Z₃)Z₄H, or —Z₅—CH₂—C(═Z₃)Z₄H.

40.-45. (canceled)

46. The compound of claim 39 wherein at least one of X₁, X₂, X₃, and X₄ is —C(═O)OH.

47. (canceled)

48. A compound having the following structure:

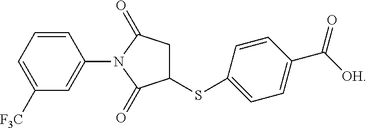

49. The compound of claim 39 wherein the compound has a structure selected from:

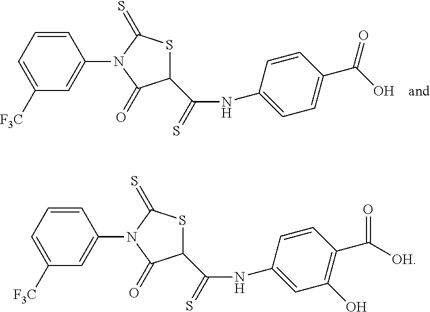

50.-55. (canceled)

56. The compound of claim 1 wherein X₅ is absent, Z₂ is O, J is S and R₅ is absent, and the compound has the following structure I(B): 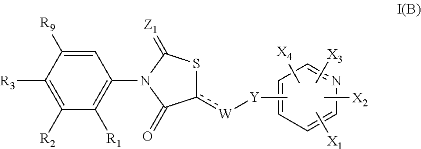
or a pharmaceutically acceptable salt, prodrug, or stereoisomer thereof, wherein
Y is —NH— or absent;
W is ═CH—, —S—, or —C(═S);
Z₁ is O or S;
R₁, R₂, R₃, and R₉ are each independently H, C₁₋₆ alkyl, halo, —CF₃, —CF₂CF₃, or —OCF₃, wherein at least one of R₁, R₂, R₃, and R₉ is —CH₃ or —CF₃; and
X₁, X₂, X₃, and X₄ are each independently H, —OH, halo, tetrazolo, —C(═O)OH, —O—C(═O)OH, or —O—CH₂—C(═O)OH.

57.-61. (canceled)

62. The compound of claim 56 wherein X₁, X₂, X₃, and X₄ is H, Y is absent, and W is ═CH— and the compound has the following structure I(B1): 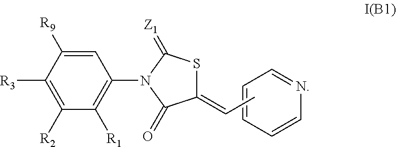
wherein
Z₁ is O or S; and
R₁, R₂, R₃, and R₉ are each independently H, —CH₃, halo, —CF₃, —CF₂CF₃, or —OCF₃, wherein at least one of R₁, R₂, R₃, and R₉ is —CH₃ or —CF₃,

63.-65. (canceled)

66. The compound of claim 62 wherein the compound has the following structure;

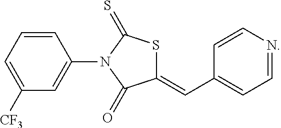

67. The compound of claim 1 wherein Z₂ is O, J is S, R₅ is absent, and the compound has the following structure I(C): 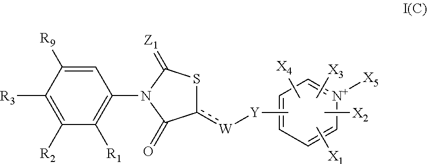
or a pharmaceutically acceptable salt, prodrug, or stereoisomer thereof, wherein
Y is —NH— or absent;
W is ═CH—, —S—, or —C(═S);
Z₁ is O or S;
R₁, R₂, R₃, and R₉ are each independently H, C₁₋₆ alkyl, halo, —CF₃, —CF₂CF₃, or —OCF₃, wherein at least one of R₁, R₂, R₃, and R₉ is —CH₃ or —CF₃;
X₁, X₂, X₃, and X₄ are each independently H, —OH, halo, —C(═O)OH, —O—C(═O)OH, or —O—CH₂—C(═O)OH; and wherein
X₅ is —O⁻, tetrazolo, —C(═O)OH, or —O—C(═O)OH.

68.-71. (canceled)

72. The compound of claim 67 wherein each of each of X₁, X₂, X₃, and X₄ is H, Y is absent, and W is ═CH— and the compound has the following structure I(C1): 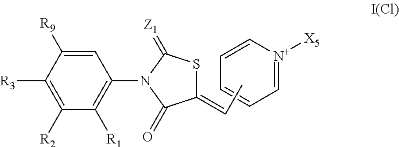
wherein
Z₁ is O or S;
R₁, R₂, R₃, and R₉ are each independently H, —CH₃, halo, —CF₃, —CF₂CF₃, or —OCF₃, wherein at least one of R₁, R₂, R₃, and R₉ is —CH₃ or —CF₃; and
X₅ is —O⁻, tetrazolo, —C(═O)OH, or —O—C(═O)OH.

73.-75. (canceled)

76. The compound of claim 72 wherein the compound has the following structure:

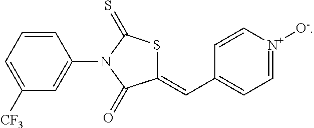

77. A compound having the following structure II: 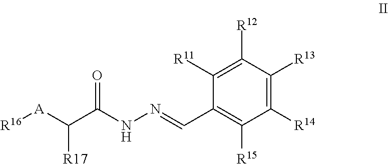
or a pharmaceutically acceptable salt, prodrug, or stereoisomer thereof, wherein
A is —O— or —NH—;
R¹¹, R¹², R¹³, R¹⁴, R¹⁵ are each the same or different and independently hydrogen, hydroxy, C₁₋₆ alkyl, C₁₋₆ alkoxy, carboxy, halo, nitro, aryl, and heteroaryl;
R¹⁶ is phenyl, heteroaryl, quinolinyl, anthracenyl, or naphthalenyl; and
R¹⁷ is. H, alkoxy, or substituted or unsubstituted aryl, and wherein
(a) A is —O— and R¹⁷ is H, and the compound has the following structure II(A): 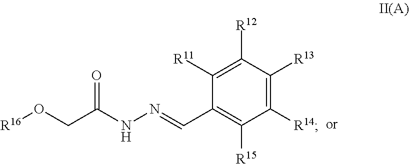
(b) A is —NH— and R¹⁷ is unsubstituted phenyl, and the compound has the following a structure II(B): 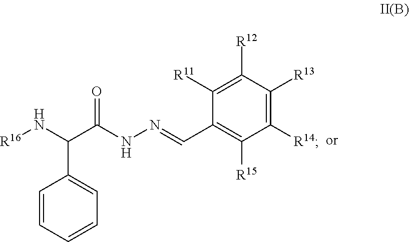
(c) when A is —NH—, R¹⁷ is substituted phenyl, wherein phenyl is substituted with halo, C₁₋₆ alkyl, C₁₋₆ alkoxy, or carboxy.

78.-80. (canceled)

81. The compound of claim 77 wherein R¹⁶ is unsubstituted phenyl, or phenyl substituted with one or more of hydroxy, methyl, chloro, or fluoro; or wherein R¹⁶ is 2-naphthalenyl, 1-naphthalenyl, 2-chlorophenyl, 4-chlorophenyl, 2,4-chlorophenyl, 4-methylphenyl, 2-anthracenyl, 7-quinolinyl, or 6-quinolinyl.

82. (canceled)

83. (canceled)

84. The compound of claim 77, wherein
R¹¹, R¹², R¹³, R¹⁴, R¹⁵ are each the same or different and independently hydrogen, hydroxy, carboxy, or halo; or
R¹¹ is H, each of R¹² and R¹⁴ is halo and each of R¹³ and R¹⁵ is hydroxy; or
R¹¹ is H, each of R¹² and R¹⁴ is halo, R¹³ is hydroxyl, and R¹⁵ is H.

85. (canceled)

86. The compound of claim 77, wherein the compound has a structure selected from:

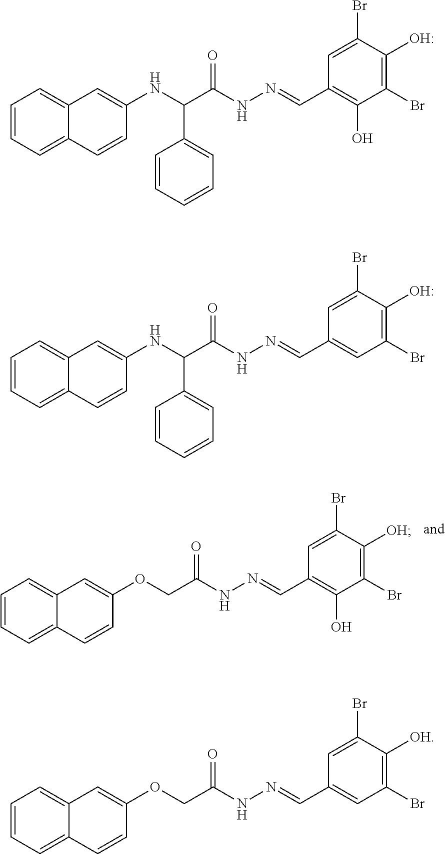

87. A pharmaceutical composition comprising a pharmaceutically suitable excipient and the compound of any one of claims 1, 3, and 77.

88. A method of inhibiting cyst formation or cyst enlargement comprising contacting (a) a cell that comprises CFTR and (b) the compound of any one of claims 1, 3, and 77, under conditions and for a time sufficient that permit the CFTR and the compound to interact, wherein the compound inhibits ion transport by CFTR.

89. A method of treating polycystic kidney disease comprising administering to subject the composition of claim 87.

90. (canceled)

91. A method of treating a disease or disorder associated with aberrantly increased ion transport by cystic fibrosis transmembrane conductance regulator (CFTR), the method comprising administering to a subject the pharmaceutical composition of claim 87, wherein ion transport by CFTR is inhibited.

92.-97. (canceled)

98. A method of inhibiting ion transport by a cystic fibrosis transmembrane conductance regulator (CFTR) comprising contacting (a) a cell that comprises CFTR and (b) the compound of any one of claims 1, 3, and 77, under conditions and for a time sufficient that permit the CFTR and the compound to interact, thereby inhibiting ion transport by CFTR.

99. (canceled)

100. A method of inhibiting cyst formation or cyst enlargement comprising contacting (a) a cell that comprises CFTR and (b) a compound that inhibits ion transport by CFTR, under conditions and for a time sufficient for the CFTR and the compound to interact, wherein the compound has the following structure:

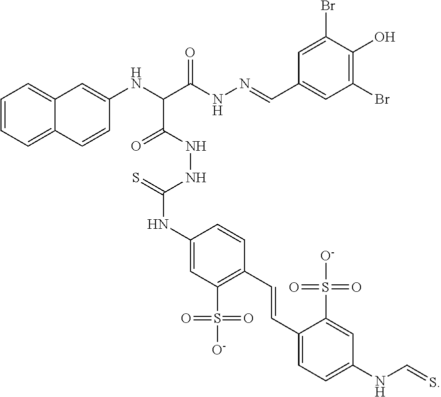

101. A method of treating polycystic kidney disease comprising administering to subject a pharmaceutical composition that comprises a pharmaceutically suitable excipient and a compound having a structure selected from:

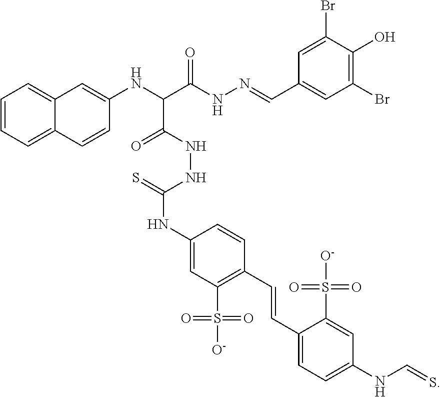

102. (canceled)

103. (canceled)
